SEQUENCE LISTING (80 sequences)

SEQ ID NO: 1 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 3 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 615 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 5 - protein, 615 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEETTILLNCAGQVKGRKPAALGEAQPTKSLEENKALKEQKKLNDLCFLKRLLQEIKTCWNKILMGTKEH

SEQ ID NO: 6 - protein, 615 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEETTILLNCAGQVKGRKPAALGEAQPTKSLEENKALKEQKKLNDLCFLKRLLQEIKTCWNKILMGTKEH

SEQ ID NO: 7 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDKHLLKVSEKTTILLNCTGQVKGRKPAALGEAQPTKSLEENKSLKEQKKLNDLCFLKRLLQEIKTCWNKILMGTKEH

SEQ ID NO: 8 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDKHLLKVSEGTTILLNCTGQVKGRKPAALGEAQPTKSLEENKSLKEQKKLNDLCFLKRLLKEIKTCWNKILMGTKEH

SEQ ID NO: 9 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMKSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLKEIKTCWNKILMGTKEH

SEQ ID NO: 10 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESKLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLKEIKTCWNKILMGTKEH

SEQ ID NO: 11 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLAVSEGTTILLNCTGQVKGRKPAALGEAQPTKSLEENKSLKEQKKLNDLCFLKRLLKEIKTCWNKILMGTKEH

SEQ ID NO: 12 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGKFDLHLLKVSEKTTILLNCTGQVKGRKPAALGEAQPTKSLEENKSLKEQKKLNDLCFLKRLLQEIKTCWNKILMGTKEH

SEQ ID NO: 13 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMKSIDQLLDSMKEIGSNCLNNEFNFFKRHICDANKEGMFLFRAARKLRQFLKMNSTGDFDLHLLKVSEKTTILLNCTGQVKGRKPAALGEAQPTKSLEENKSLKEQKKLNDLCFLKRLLQEIKTCWNKILMGTKEH

SEQ ID NO: 14 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESALMVSIDQLLDSMKEIGSNCLNNEFNFFKRHICDANKEGMFLFRAARKLRQFLKMNSTGDFDLHLLKVSEKTTILLNCTGQVKGRKPAALGEAQPTKSLEENKSLKEQKKLNDLCFLKRLLQEIKTCWNKILMGTKEH

SEQ ID NO: 15 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESALMVSIDQLLDSMKEIGSNCLNNEFNFFKRHICDANKEGMFLFRAARKLRQFLKMNSTGDFDLHLLAVSEGTTILLNCTGQVKGRKPAALGEAQPTKSLEENKSLKEQKKLNDLCFLKRLLQEIKTCWNKILMGTKEH

SEQ ID NO: 16 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEK

SEQ ID NO: 17 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKKH

SEQ ID NO: 18 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKKNDLCFLKRLLQEIKTCWNKILMGTKEH

SEQ ID NO: 19 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAAQGEAQPTKSQEENKSLKEQKKLNDLCFLKRLLQEIKTCWNKILMGTKEH

SEQ ID NO: 20 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSKEENKSLKEQKKLNDLCFLKRLLQEIKTCWNKILMGTKEH

SEQ ID NO: 21 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAAKGEAQPTKSLEENKSLKEQKKLNDLCFLKRLLQEIKTCWNKILMGTKEH

SEQ ID NO: 22 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLKEVSEETTILLNCTGQVKGRKPAALGEAQPTKSLEENKSLKEQKKLNDLCFLKRLLQEIKTCWNKILMGTKEH

SEQ ID NO: 23 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFKKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 24 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEKGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 25 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDKHLLKVSEETTILLNCTGQVKGRKPAALGEAQPTKSLEENKSLKEQKKLNDLCFLKRLLQEIKTCWNKILMGTKEH

SEQ ID NO: 26 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYKSVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 27 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLQVSEQTTILLNCTGQVKGRKPAALGEAQPTKSLEENKSLKEQKKLNDLCFLKRLLQEIKTCWNKILMGTKEH

SEQ ID NO: 28 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEQTTILLNCTGQVKGRKPAALGEAQPTKSLEENKSLKEQKKLNDLCFLKRLLQEIKTCWNKILMGTKEH

SEQ ID NO: 29 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLQVSEETTILLNCTGQVKGRKPAALGEAQPTKSLEENKSLKEQKKLNDLCFLKRLLQEIKTCWNKILMGTKEH

SEQ ID NO: 30 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKNGTQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSNETNKSLKEQKKLNDLCFLKRLLQEIKTCWNKILMGTKEH

SEQ ID NO: 31 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKNGTQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENGTLKEQKKLNDLCFLKRLLQEIKTCWNKILMGTKEH

SEQ ID NO: 32 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNQNFTFFKRHICDANKEGMFLFRAARKLRQFLKMNSTGDFDLHLLEVSEETTILLNCTGQVKGRKPAALGEAQPNKTLEENKSLKEQKKLNDLCFLKRLLQEIKTCWNKILMGTKEH

SEQ ID NO: 33 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNNFTFFKRHICDANKEGMFLFRAARKLRQFLKMNSTGDFDLHLLEVSEETTILLNCTGQVKGRKPAALGNATSTKSLEENKSLKEQKKLNDLCFLKRLLQEIKTCWNKILMGTKEH

SEQ ID NO: 34 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKNGTQYESVLMVSIDQLLDSMKEIGSNCLNNEFNFFKRHICDANKNGTFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 35 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCNITGKDGKQYESVLMVSIDQLLDSMKEIGSNCLNNEFNFFKRHICDANKNGTFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 36 - protein, 621 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTLNGTSTST

SEQ ID NO: 37 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSNETNKSLKEQKKLNDLCFLKRLLQEIKTCWNKILMGTKEH

SEQ ID NO: 38 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPNKTLEENKSLKEQKKLNDLCFLKRLLQEIKTCWNKILMGTKEH

SEQ ID NO: 39 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGNATSTKSLEENKSLKEQKKLNDLCFLKRLLQEIKTCWNKILMGTKEH

SEQ ID NO: 40 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKNGTFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 41 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICNATKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 42 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNQNFTFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 43 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNNFTFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 44 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCNITGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 45 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKNGTQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 46 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 47 - protein, 616 aa, synthetic construct; identical to SEQ ID NO: 3

SEQ ID NO: 48 - protein, 615 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 49 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 50 - protein, 616 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 51 - protein, 615 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 52 - protein, 616 aa, synthetic construct; identical to SEQ ID NO: 46

SEQ ID NO: 53 - protein, 218 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 449 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 55 - protein, 616 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 56 - protein, 616 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYESVLMVSIDQLLDSMKEIGSNCLNNEFNFFKRHICDANKEGMFLFRAARKLRQFLKMNSTGKFDLHLLKVSEKTTILLNCTGQVKGRKPAALGEAQPTKSLEENKSLKEQKKLNDLCFLKRLLQEIKTCWNKILMGTKEH

SEQ ID NO: 57 - protein, 616 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGDCDIEGKDGKQYKSVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 58 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 447 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 613 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 61 - protein, 219 aa, synthetic construct
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 612 aa, synthetic construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 63 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 64 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 65 - protein, 152 aa, synthetic construct
DCDIEGKDGKQYESVLMVSIDQLLDSMKEIGSNCLNNEFNFFKRHICDANKEGMFLFRAARKLRQFLKMNSTGKFDLHLLKVSEKTTILLNCTGQVKGRKPAALGEAQPTKSLEENKSLKEQKKLNDLCFLKRLLQEIKTCWNKILMGTKEH

SEQ ID NO: 66 - protein, 152 aa, synthetic construct
DCDIEGKDGKQYKSVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 67 - protein, 5 aa, synthetic construct
SYTMS

SEQ ID NO: 68 - protein, 17 aa, synthetic construct
AISGGGRDIYYPDSVKG

SEQ ID NO: 69 - protein, 11 aa, synthetic construct
LTGRVYFALDS

SEQ ID NO: 70 - protein, 15 aa, synthetic construct
KASESVDTSDNSFIH

SEQ ID NO: 71 - protein, 7 aa, synthetic construct
RASTLES

SEQ ID NO: 72 - protein, 9 aa, synthetic construct
QQNYDVPWT

SEQ ID NO: 73 - protein, 120 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSS

SEQ ID NO: 74 - protein, 111 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIK

SEQ ID NO: 75 - protein, 15 aa, synthetic construct
GGSGGGGSGGGGSGG

SEQ ID NO: 76 - protein, 218 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 77 - protein, 443 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIEPKSCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 78 - protein, 610 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIEPKSCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGDCDIEGKDGKQYKSVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 79 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 80 - protein, 443 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

(19) United States
(12) Patent Application Publication
(10) Pub. No.: US 2025/0361279 A1
(43) Pub. Date: Nov. 27, 2025

(54) New Interleukin-7 Immunoconjugates

(71) Applicant: Hoffmann-La Roche Inc., Little Falls, NJ (US)

(72) Inventors: Alejandro CARPY GUTIERREZ CIRLOS, Penzberg (DE); Laura CODARRI DEAK, Au (CH); Harald DUERR, Starnberg (DE); Greta DURINI, Zürich (CH); Stefan FROST, Bad Heilbrunn (DE); Lukasz Antoni KACPRZYK, Weilheim in Oberbayern (DE); Christian KLEIN, Zürich (CH); Johann KOLL, Schwabhausen (DE); Ekkehard MOESSNER, Kreuzlingen (CH); Cindy SCHULENBURG, Zürich (CH); Pablo UMAÑA FERNÁNDEZ, Lachen (CH)

(73) Assignee: Hoffmann-La Roche Inc., Little Falls, NJ (US)

(21) Appl. No.: 19/092,753

(22) Filed: Mar. 27, 2025

(30) Foreign Application Priority Data
Mar. 27, 2024 (EP) 24167077.7

(51) Int. Cl. C07K 14/54; A61K 38/00; A61K 39/00; A61P 35/00; C07K 16/28

(52) U.S. Cl. CPC C07K 14/5418; A61P 35/00; C07K 16/2818; A61K 38/00; A61K 2039/505; C07K 2317/75; C07K 2317/92; C07K 2317/94; C07K 2319/33; C07K 2319/75

(57) ABSTRACT

The present invention generally relates to mutant interleukin-7 polypeptides, immunoconjugates, particularly immunoconjugates comprising a mutant interleukin-7 polypeptide and an antibody that binds to PD-1. In addition, the invention relates to polynucleotide molecules encoding the mutant interleukin-7 polypeptides or the immunoconjugates, vectors and host cells comprising such polynucleotide molecules. The invention further relates to methods for producing the mutant interleukin-7 polypeptides, immunoconjugates, pharmaceutical compositions comprising the same, and uses thereof.

### CROSS REFERENCE TO RELATED APPLICATIONS

This application claims benefit to European Patent Application No. 24167077.7, filed Mar. 27, 2024, the contents of which are hereby expressly incorporated by reference in its entirety.

### SEQUENCE LISTING

This application contains a Sequence Listing which has been submitted electronically in XML format and is hereby incorporated by reference in its entirety. Said XML copy, created on Mar. 24, 2025 is named P39737-US_Sequence Listing.xml and is 108,682 bytes in size.

### FIELD OF THE INVENTION

The present invention generally relates to mutant interleukin-7 polypeptides, immunoconjugates, particularly immunoconjugates comprising a mutant interleukin-7 polypeptide and an antibody that binds to PD-1. In addition, the invention relates to polynucleotide molecules encoding the mutant interleukin-7 polypeptide or immunoconjugates, vectors and host cells comprising such polynucleotide molecules. The invention further relates to methods for producing the mutant interleukin-7 polypeptide or immunoconjugates, pharmaceutical compositions comprising the same, and uses thereof.

### BACKGROUND

Interleukin-7 (IL-7) is a cytokine mainly secreted by stromal cells in lymphoid tissues. It is involved in the maturation of lymphocytes, e.g. by stimulating the differentiation of multipotent hematopoetic stem cells to lymphoblasts. IL-7 is essential for T-cell development and survival, as well as for mature T-cell homeostasis. A lack of IL-7 causes immature immune cell arrest (Lin J. et al. (2017), Anticancer Res. 37 (3): 963-967).

IL-7 binds to the IL-7 receptor, which is composed of the IL-7R alpha chain (IL-7Rα, CD127) as well as the common gamma chain (γc, CD132, IL-2Rγ), that is mutual to the interleukines IL-2, IL-4, IL-7, IL-9, IL-15 and IL-21 (Rochman Y. et al., (2009) Nat Rev Immunol. 9:480-490). Whereas γc is expressed by most haematopoietic cells, IL-7Ra is almost exclusively expressed by cells of the lymphoid lineage (Mazzucchelli R. and Durum S. K. (2007) Nat Rev Immunol. 7 (2): 144-54). IL-7Rα is found on the surface of T cells across their differentiation from naïve to effector while its expression is reduced on terminally differentiated T cells and is virtually absent from the surface of regulatory T cells. IL-7Rα mRNA and protein expression levels are negatively regulated by IL-2, therefore IL-7Rα is downregulated in recently activated T cells expressing the IL-2Rα (CD25) (Xue H. H, et al. 2002, PNAS. 99 (21): 13759-64), this mechanism ensures the IL-2 mediated rapid clonal expansion of recently primed T cells while IL-7 role is to equally maintain all T cell clones. IL-7Rα has also been recently described on a newly characterized precursor population of CD8 T cells, TCF-1+ PD-1+ stem-like CD8 T cells, which is found in the tumor of cancer patients responding to PD-1 blockade (Hudson et al., 2019, Immunity 51, 1043-1058; Im et al., PNAS, vol. 117, no. 8, 4292-4299; Siddiqui et al., 2019, Immunity 50, 195-211; Held et al., Sci., Transl. Med. 11; eaay6863 (2019); Vodnala and Restifo, Nature, Vol 576, 19/26 Dec. 2019). Although, until today, there are no scientific descriptions of the effect of IL-7 on the stem like CD8 T cells, IL-7 could be used to expand this population of tumor reactive T cells in order to increase the number of patients responding to check point inhibitors.

IL-7, IL-7Rα and γc form a ternary complex, which signals over the JAK/STAT (Janus kinase (JAK)-signal transducer and activator of transcription (STAT)) pathway as well as the PI3K/Akt (Phosphatidylinositol 3-kinase (PI3K), serine/threonine protein kinase, protein kinase B (AKT)) signaling cascade, leading to the development and homeostasis of B- and T-cells (Niu N. and Qin X. (2013) Cell Mol Immunol. 10 (3): 187-189, Jacobs et al., (2010), J Immunol. 184 (7): 3461-3469).

IL-7 is a 25 kDa 4-helix bundle, monomeric protein. The helix length varies from 13 to 22 amino acids, which is similar to the helix length of other common gamma chain (γc, CD132, IL-2Ry) binding interleukines. However, IL-7 shows a unique turn motif in the A helix, which was shown to stabilize the IL-7/IL-7Rα interaction (McElroy, C. A. et al., (2009) Structure 17:54-65). Whereas the A helix interacts with both receptor chains IL-7Rα and γc, the C helix interacts predominantly with IL-7Rα and the D helix with the γc chain (sequence and structural alignments based on PDB: 3DI2 and PDB: 2ERJ). Variant IL-7s with modifications to reduce heterogeneity and/or reduced affinity/potency have been described in WO 2020/127377 A1 and WO 2020/236655 A1.

Programmed cell death protein 1 (PD-1 or CD279) is an inhibitory member of the CD28 family of receptors, that also includes CD28, CTLA-4, ICOS and BTLA. PD-1 is a cell surface receptor and is expressed on activated B cells, T cells, and myeloid cells (Okazaki et al (2002) Curr. Opin. Immunol. 14:391779-82; Bennett et al. (2003) J Immunol 170:711-8). The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Two ligands for PD-1 have been identified, PD-L1 and PD-L2, that have been shown to downregulate T cell activation upon binding to PD-1 (Freeman et al (2000) J Exp Med 192:1027-34; Latchman et al (2001) Nat Immunol 2:261-8; Carter etal (2002) Eur J Immunol 32:634-43). Both PD-L1 and PD-L2 are B7 homologs that bind to PD-1, but do not bind to other CD28 family members. One ligand for PD-1, PD-L1 is abundant in a variety of human cancers (Dong et al (2002) Nat. Med 8:787-9). The interaction between PD-1 and PD-LI results in a decrease in tumor infiltrating lymphocytes, a decrease in T-cell receptor mediated proliferation allowing immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-LI, and the effect is additive when the interaction of PD-1 with PD-L2 is blocked as well (Iwai et al. (2002) Proc. Nat 7. Acad. ScL USA 99:12293-7; Brown et al. (2003) J. Immunol. 170:1257-66).

Antibodies that bind to PD-1 are described e.g. in WO 2017/055443 A1. Immunconjugates targeting PD1 and comprising an interleukin-7 mutein are described e.g. in WO 2021/209402 A2, WO 2023/062050 A1 and WO 2023/062048 A1.

### SUMMARY OF THE INVENTION

The present invention provides a novel approach of targeting a mutant form of IL-7 with advantageous properties for immunotherapy directly to immune effector cells, such as cytotoxic T lymphocytes, rather than tumor cells, through conjugation of the mutant IL-7 polypeptide to an antibody that binds to PD-1. This results in cis-delivery of the IL-7 mutant to PD-1 expressing immune subsets, especially tumor reactive T cells e.g. CD8+ PD1+ TCF+ T cell subsets and their progeny.

The IL-7 mutants used in the present invention have been designed to overcome the problems associated with cytokine immunotherapy, in particular toxicity caused by the induction of VLS, tumor tolerance caused by the induction of AICD, and immunosuppression caused by activation of Treg cells. In addition to circumventing escape of tumors from tumor-targeting as mentioned above, targeting of the IL-7 mutant to immune effector cells may further increase the preferential activation of tumor specific CTLs over immunosuppressive Treg cells due to lower PD-1 and IL-7Rα expressing levels on Tregs than CTLs. By using an antibody that binds to PD-1, the suppression of T-cell activity induced by the interaction of PD-1 with its ligand PD-L1 may additionally be reversed, thus further enhancing the immune response.

In one aspect, the invention provides a mutant interleukin-7 (IL-7) polypeptide the amino acid substitutions E13K and G85E, wherein the numbering is relative to the human IL-7 sequence of SEQ ID NO: 64.

In one aspect, the mutant IL-7 polypeptide has a reduced binding affinity to IL-7Rα as compared to a reference IL-7 polypeptide which is identical to the mutant IL-7 polypeptide except that the reference IL-7 polypeptide has E at position 13 and G at position 85, wherein the numbering is relative to the sequence of SEQ ID NO: 64. In one aspect, the invention provides a mutant interleukin-7 (IL-7) polypeptide, comprising at least two amino acid substitutions at the position of E13 and G85 of human IL-7 according to SEQ ID NO: 64, wherein the amino acid substitution reduces the binding affinity of the mutant interleukin-7 polypeptide to IL-7Rα compared to an interleukin-7 polypeptide comprising SEQ ID NO: 64, wherein the at least two amino acid substitutions are E13K and G85E.

In one aspect, the mutant IL-7 polypeptide comprises an amino acid sequence that is at least about 70% identical to SEQ ID NO: 66. In one aspect, the mutant interleukin-7 polypeptide comprises the amino acid sequence according to SEQ ID NO: 66.

In one aspect, the invention provides a protein comprising a mutant IL-7 polypeptide as described herein.

In a further aspect, the invention provides an immunoconjugate comprising (i) a mutant IL-7 polypeptide as described herein and (ii) an antibody. In one aspect, said antibody binds to PD-1. In one aspect, the antibody comprises (a) a heavy chain variable region (VH) comprising a CDR-H1 comprising the amino acid sequence of SEQ ID NO:67, a CDR-H2 comprising the amino acid sequence of SEQ ID NO:68, a CDR-H3 comprising the amino acid sequence of SEQ ID NO:69, and (b) a light chain variable region (VL) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In a further aspect, the antibody comprises (a) a heavy chain variable region (VH) comprising an amino acid sequence that is at least about 95%, at least about 96%, at least about 97%, at least about 98%, at least about 99% or 100% identical to the amino acid sequence of SEQ ID NO:73, and (b) a light chain variable region (VL) comprising an amino acid sequence that is at least about 95%, at least about 96%, at least about 97%, at least about 98%, at least about 99% or 100% identical to an amino acid sequence of SEQ ID NO:74.

In a further aspect, the antibody comprises (a) a heavy chain variable region (VH) comprising an amino acid sequence that is identical to the amino acid sequence of SEQ ID NO:73, and (b) a light chain variable region (VL) comprising an amino acid sequence that is to an amino acid sequence of SEQ ID NO:74.

In one aspect, the immunoconjugate comprises not more than one IL-7 polypeptide.

In one aspect, the immunoconjugate comprises not more than one mutant IL-7 polypeptide.

In another aspect, the antibody comprises an Fc domain composed of a first and a second subunit. In one aspect, the Fc domain is an IgG class, particularly an IgG1 subclass, Fc domain. In a further aspect, the Fc domain is a human Fc domain.

In one aspect, the antibody is an IgG class, particularly an IgG1 subclass immunoglobulin.

In one aspect, the Fc domain comprises a modification promoting the association of the first and the second subunit of the Fc domain. In one aspect, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. In another aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In yet a further aspect, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

In one aspect, the mutant IL-7 polypeptide is fused at its amino-terminal amino acid to the carboxy-terminal amino acid of one of the subunits of the Fc domain, particularly the first subunit of the Fc domain, optionally through a linker peptide. In one aspect, the linker peptide has the amino acid sequence of SEQ ID NO: 75.

In another aspect, the Fc domain comprises one or more amino acid substitution that reduces binding to an Fc receptor, particularly an Fcγ receptor, and/or effector function, particularly antibody-dependent cell-mediated cytotoxicity (ADCC). In one aspect, said one or more amino acid substitution is at one or more position selected from the group of L234, L235, and P329 (Kabat EU index numbering). In one aspect, each subunit of the Fc domain comprises the amino acid substitutions L234A, L235A and P329G (Kabat EU index numbering).

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 57.

In one aspect, the invention provides an immunoconjugate comprising two polypeptides, each comprising an amino acid sequence that is at least about 80%, 85%, 90%, 95%, 96%, 97%, 98%, 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 57.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 56.

In one aspect, the invention provides an immunoconjugate comprising two polypeptides, each comprising an amino acid sequence that is at least about 80%, 85%, 90%, 95%, 96%, 97%, 98%, 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 56.

In one aspect, the invention provides an immunoconjugate comprising a polypeptide comprising an amino acid sequence that is identical to the sequence of SEQ ID NO: 53, a polypeptide comprising an amino acid sequence that is identical to the sequence of SEQ ID NO: 54, and a polypeptide comprising an amino acid sequence that is identical to the sequence of SEQ ID NO: 57.

In one aspect, the immunoconjugate essentially consists of a mutant IL-7 polypeptide and an IgG1 immunoglobulin molecule, joined by a linker sequence. In another aspect, the immunoconjugate essentially consists of a mutant IL-7 polypeptide and an IgG1 immunoglobulin molecule, joined by a linker of SEQ ID NO: 75.

In one aspect, one or more isolated polynucleotide encoding a mutant IL-7 polypeptide of the invention or a protein comprising such mutant IL-7 polypeptide, such as an immunoconjugate, are provided. In one aspect, the invention provides one or more vector, particularly expression vector, comprising the polynucleotide(s) of the invention. In one aspect, the invention provides a host cell comprising the polynucleotide(s) or the vector(s) of the invention.

In one aspect, a method of producing a mutant IL-7 polypeptide or a protein comprising such mutant IL-7 polypeptide, such as an immunoconjugate comprising a mutant IL-7 polypeptide and an antibody that binds to PD-1, is provided comprising (a) culturing the host cell under conditions suitable for the expression of the mutant IL-7 polypeptide or the protein, and optionally (b) recovering the mutant IL-7 polypeptide or the protein. In one aspect, the invention provides a mutant IL-7 polypeptide or a protein comprising such mutant IL-7 polypeptide, such as an immunoconjugate comprising a mutant IL-7 polypeptide and an antibody that binds to PD-1, produced by said method.

In one aspect, the invention provides a pharmaceutical composition comprising a mutant IL-7 polypeptide or a protein comprising such mutant IL-7 polypeptide, such as a immunoconjugate of the invention, and a pharmaceutically acceptable carrier.

In one aspect, the invention provides a mutant IL-7 polypeptide or a protein comprising such mutant IL-7 polypeptide, such as an immunoconjugate of the invention, for use as a medicament.

In one aspect, the invention provides a mutant IL-7 polypeptide or a protein comprising such mutant IL-7 polypeptide, such as an immunoconjugate of the invention, for use in the treatment of a disease. In one aspect, said disease is cancer.

In a further aspect, the invention provides the use of the mutant IL-7 polypeptide or a protein comprising such mutant IL-7 polypeptide, such as the immunoconjugate of the invention, in the manufacture of a medicament for the treatment of a disease. In one aspect, said disease is cancer.

In one aspect, the invention provides a method of treating a disease in an individual, comprising administering to said individual a therapeutically effective amount of a composition comprising the mutant IL-7 polypeptide or a protein comprising such mutant IL-7 polypeptide, such as the immunoconjugate of the invention, in a pharmaceutically acceptable form. In one aspect, said disease is cancer.

In one aspect, the invention provides a method of stimulating the immune system of an individual, comprising administering to said individual an effective amount of a composition comprising the mutant IL-7 polypeptide or a protein comprising such mutant IL-7 polypeptide, such as the immunoconjugate of the invention, in a pharmaceutically acceptable form.

### BRIEF DESCRIPTION OF THE DRAWINGS

FIG. 1: Comparison of analytical size exclusion chromatograms of PD1-IL7v variants, generated via fed-batch production. PD1-IL7var21_fully glycosylated corresponds to P1AG3724, PD1-IL7var21_partially glycosylated corresponds to P1AG3725 and PD1-IL7var18_21_partially glycosylated corresponds to P1AG3727.

FIG. 2A: Comparison of preparative size exclusion chromatograms of different PD1-IL7v variants (Category A, table 4), generated via small-scale production. P1AH4515 contains the IL7 wild-type sequence and serves as a positive control, P1AG3727 corresponds to PD1-IL7var18_21_partially glycosylated, P1AG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 2B: Comparison of preparative size exclusion chromatograms of different PD1-IL7v variants (Category B, table 4), generated via small-scale production. P1AH4515 contains the IL7 wild-type sequence and serves as a positive control, P1AG3727 corresponds to PD1-IL7var18_21_partially glycosylated, P1AG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 2C: Comparison of preparative size exclusion chromatograms of different PD1-IL7v variants (Category C, table 4), generated via small-scale production. P1AH4515 contains the IL7 wild-type sequence and serves as a positive control, P1AG3727 corresponds to PD1-IL7var18_21 partially glycosylated, P1AG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 2D: Comparison of preparative size exclusion chromatograms of different PD1-IL7v variants (Category D, table 4), generated via small-scale production. P1AH4515 contains the IL7 wild-type sequence and serves as a positive control, P1AG3727 corresponds to PD1-IL7var18_21 partially glycosylated, P1AG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 2E: Comparison of preparative size exclusion chromatograms of different PD1-IL7v variants (Category D, table 4), generated via small-scale production. P1AH4515 contains the IL7 wild-type sequence and serves as a positive control, P1AG3727 corresponds to PD1-IL7var18_21_partially glycosylated, P1AG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 2F: Comparison of preparative size exclusion chromatograms of different PD1-IL7v variants (Category E, table 4), generated via small-scale production. P1AH4515 contains the IL7 wild-type sequence and serves as a positive control, P1AG3727 corresponds to PD1-IL7var18_21_partially glycosylated, PAG3048 and P1AH4513 corresponds to PD1-IL7var18_21_fully glycosylated and differ in linker sequence. P1AH4513 served as a starting point for protein engineering efforts aiming at discovering variants with improved CMC properties. All molecules, except P1AG3727, are generated fully glycosylated, meaning all glycosylation consensus sequences are intact.

FIG. 3A: New double mutant combinations from existing IL-7 mut-Frequency. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 5 donors.

FIG. 3B: New double mutant combinations from existing IL-7 mut-Frequency. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 5 donors.

FIG. 3C: New double mutants around position 82/85-Frequency. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 5 donors.

FIG. 4: Treg suppression assay with PD1 (0376)-IL7v. Percentage of T_{reg}-mediated suppression of granzyme B production by T_{conv} in a 5 days coculture in presence or absence of the indicated immunoconjugates. Median of 5 donors. P is calculated using one-way ANOVA (*p<0.05, **p<0.01, ***p<0.001, ****p<0.0001).

FIG. 5A: Transient Expression-CisTrans assay-De-risked PD1-IL7v2.6. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 5 donors.

FIG. 5B. Transient Expression-CisTrans assay-De-risked PD1-IL7v2.2. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 6 donors.

FIG. 6: Transient Expression-Treg suppression assay-de-immunized PD1-IL7v2.6. Percentage of Treg-mediated suppression of granzyme B production by T_{conv} in a 5 days coculture in presence or absence of the indicated immunoconjugates. Median of 12 donors. P is calculated using one-way ANOVA (*p<0.05, **p<0.01, ***p<0.001, ****p<0.0001).

FIG. 7: Chromatographic pattern of N-Oligosaccharides from P1AI5585 and PIAI5587 after high titer recombinant expression in stably transfected CHO cells compared to transient expression at low titer (1AI5587-270.

FIG. 8: Binding of P1AI5585 and P1AI5587 to 14 different non-specific antigens.

FIG. 9: ExpII-Stable material-CisTrans assay-Frequency. IL-7R signaling (STAT5-P) in PD1-blocked and PD-1 expressing CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 8 donors.

FIG. 10. ExpII-Stable material-Fresh vs activated-Frequency. IL-7R signaling (STAT5-P) in activated and freshly isolated CD4 T cells cultured together. IL-7R signaling (STAT5-P) depicted as frequency of STAT5-P in human PD1+ (solid line) and PD-1 pre-blocked (dotted line) CD4 T cells upon 12 min exposure to PD1-IL7 immunocognugates. Mean±SEM of 6 donors.

FIG. 11A: Dose-response binding of the different constructs to naïve, stem-like, effector and bystander T cells within CD4 T cells. Frequency of bound molecule to subsets of CD4 T cells, defined by their expression of PD-1 and TCF-1 in naïve (TCF-1⁺ PD-1⁻), stem-like (PD-1⁺ TCF-1⁺), effector (PD-1⁺ TCF-1⁻) and bystander (PD-1⁻ TCF-1⁻), upon 30 min exposure to PD1-IL7 immunoconjugates followed by 30 min incubation with anti-PGLALA antibody. Mean±SEM of 2 donors.

FIG. 11B: Dose-response binding of the different constructs to naïve, stem-like, effector and bystander T cells within CD8 T cells. Frequency of bound molecule to subsets of CD8 T cells, defined by their expression of PD-1 and TCF-1 in naïve (TCF-1⁺ PD-1⁻), stem-like (PD-1⁺ TCF-1⁺), effector (PD-1⁺ TCF-1⁻) and bystander (PD-1⁻ TCF-1⁻), upon 30 min exposure to PD1-IL7 immunoconjugates followed by 30 min incubation with anti-PGLALA antibody. Mean±SEM of 2 donors.

FIG. 12: Tumor growth inhibition of murinized P1AI5587 and murinized Pembrolizumab in a Panc02 pancreatic subcutaneous syngeneic model. The Panc02 pancreatic carcinoma cell line was injected subcutaneously into Black 6-huPD1-huIL2RBG transgenic mice. Tumor size was measured using a caliper. Therapy started when tumors reached 200 mm3. The amount of antibodies injected per mouse was 2 mg/kg for murinized P1AI5587 and murinized Pembrolizumab qw. The treatment lasted 2 weeks. The murinized P1AI5587mediated significant superior efficacy in terms of tumor growth inhibition compared to vehicle group and murinized standard of care Pembrolizumab.

### DETAILED DESCRIPTION OF THE INVENTION

### Definitions

Terms are used herein as generally used in the art, unless otherwise defined in the following.

The term “about” as used herein in connection with a specific value (e.g., percentage of sequence identity) shall refer to such specific value and any insubstantial variation thereof, e.g., a variation of +/−10%, +/−5% or +/−1% of such specific value. In some embodiments, it refers to the specific value without any variation thereof.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g. reduced binding to IL-7Rα and/or IL-2Rγ. Amino acid sequence deletions and insertions include amino- and/or carboxy-terminal deletions and insertions of amino acids. An example of a terminal deletion is the deletion of the residue in position 1 of full-length human IL-7. Preferred amino acid mutations are amino acid substitutions. For the purpose of altering e.g. the binding characteristics of an IL-7 polypeptide, non-conservative amino acid substitutions, i.e. replacing one amino acid with another amino acid having different structural and/or chemical properties, are particularly preferred. Preferred amino acid substitutions include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

IL-7 binds to the IL-7 receptor, which is composed of the IL-7R alpha chain (also referred to as IL-7Ralpha, IL-7Rα, IL7Rα, IL-7a, IL7Ra or CD127 herein) as well as the common gamma chain (also referred to as γc, CD132, IL-2Rgamma, IL-2Rg, IL2Rg, IL-2Rγ or IL2Rγ herein), that is mutual to the interleukines IL-2, IL-4, IL-7, IL-9, IL-15 and IL-21 (Rochman Y. et al., (2009) Nat Rev Immunol. 9:480-490).

The affinity of the mutant or wild-type IL-7 polypeptide for the IL-7 receptor can be determined in accordance with the method set forth in the WO 2012/107417 by surface plasmon resonance (SPR), using standard instrumentation such as a BIAcore instrument (GE Healthcare) and receptor subunits such as may be obtained by recombinant expression (see e.g. Shanafelt et al., Nature Biotechnol 18, 1197-1202 (2000)). Alternatively, binding affinity of IL-7 mutants for the IL-7 receptor may be evaluated using cell lines known to express one or the other such form of the receptor. Specific illustrative and exemplary embodiments for measuring binding affinity are described hereinafter.

The term “interleukin-7” or “IL-7” as used herein, refers to any native IL-7 from any vertebrate source, including mammals such as primates (e.g. humans) and rodents (e.g., mice and rats), unless otherwise indicated. The term encompasses unprocessed IL-7 as well as any form of IL-7 that results from processing in the cell. The term also encompasses naturally occurring variants of IL-7, e.g. splice variants or allelic variants. The amino acid sequence of an exemplary human IL-7 is shown in SEQ ID NO: 64.

The term “IL-7 mutant” or “mutant IL-7 polypeptide” as used herein is intended to encompass any mutant forms of various forms of the IL-7 molecule including full-length IL-7, truncated forms of IL-7 and forms where IL-7 is linked to another molecule such as by fusion or chemical conjugation. “Full-length” when used in reference to IL-7 is intended to mean the mature, natural length IL-7 molecule. For example, full-length human IL-7 refers to a molecule that has a polpypetide sequence according to SEQ ID NO: 64. The various forms of IL-7 mutants are characterized in having at least one amino acid mutation affecting the interaction of IL-7 with IL7Ralpha and/or IL2Rgamma. This mutation may involve substitution, deletion, truncation or modification of the wild-type amino acid residue normally located at that position. Mutants obtained by amino acid substitution are preferred. Unless otherwise indicated, an IL-7 mutant may be referred to herein as a mutant IL-7 peptide sequence, a mutant IL-7 polypeptide, a mutant IL-7 protein, a mutant IL-7 analog or a IL-7 variant.

Designation of various forms of IL-7 is herein made with respect to the sequence shown in SEQ ID NO: 64. Various designations may be used herein to indicate the same mutation. For example, a mutation from Valine at position 15 to Alanine can be indicated as 15A, A15, A15, V15A, or Val15Ala.

By a “human IL-7 molecule” as used herein is meant an IL-7 molecule comprising an amino acid sequence that is at least about 90%, at least about 91%, at least about 92%, at least about 93%, at least about 94%, at least about 95% or at least about 96% identical to the human IL-7 sequence of SEQ ID NO:64. Particularly, the sequence identity is at least about 95%, more particularly at least about 96%. In particular embodiments, the human IL-7 molecule is a full-length IL-7 molecule.

As used herein, a “wild-type” form of IL-7 is a form of IL-7 that is otherwise the same as the mutant IL-7 polypeptide except that the wild-type form has a wild-type amino acid at each amino acid position of the mutant IL-7 polypeptide. For example, if the IL-7 mutant is the full-length IL-7 (i.e. IL-7 not fused or conjugated to any other molecule), the wild-type form of this mutant is full-length native IL-7. If the IL-7 mutant is a fusion between IL-7 and another polypeptide encoded downstream of IL-7 (e.g. an antibody chain) the wild-type form of this IL-7 mutant is IL-7 with a wild-type amino acid sequence, fused to the same downstream polypeptide. Furthermore, if the IL-7 mutant is a truncated form of IL-7 (the mutated or modified sequence within the non-truncated portion of IL-7) then the wild-type form of this IL-7 mutant is a similarly truncated IL-7 that has a wild-type sequence. For the purpose of comparing IL-7 receptor binding affinity, IL-7 receptor binding or biological activity of various forms of IL-7 mutants to the corresponding wild-type form of IL-7, the term wild-type encompasses forms of IL-7 comprising one or more amino acid mutation that does not affect IL-7 receptor binding compared to the naturally occurring, native IL-7. In certain embodiments according to the invention the wild-type IL-7 polypeptide to which the mutant IL-7 polypeptide is compared comprises the amino acid sequence of SEQ ID NO: 64.

By “regulatory T cell” or “T_{reg} cell” a specialized type of CD4⁺ T cell is meant that can suppress the responses of other T cells, called peripheral tolerance. T_{reg} cells are characterized by elevated expression of the α-subunit of the IL-2 receptor (CD25), low or absent IL-7Rα (CD127) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. As used herein, the term “effector cells” refers to a population of lymphocytes which survival and/or homeostasis are affected by IL-7. Effector cells include memory CD4+ and CD8+ cells and recently primed T cells including tumor reactive stem-like T cells.

As used herein, the term “PD1”, “human PD1”, “PD-1” or “human PD-1” (also known as Programmed cell death protein 1, or Programmed Death 1) refers to the human protein PD1. See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a KD value of the binding affinity for binding to human PD-1 of ≤1 μM, ≤100 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M). In one embodiment, the KD value of the binding affinity is determined in a Surface Plasmon Resonance assay using the Extracellular domain (ECD) of human PD-1 (PD-1-ECD) as antigen.

By “specific binding” is meant that the binding is selective for the antigen and can be discriminated from unwanted or non-specific interactions. The ability of an antibody to bind to a specific antigen (e.g. PD-1) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody to an unrelated protein is less than about 10% of the binding of the antibody to the antigen as measured, e.g., by SPR. The antibody comprised in the immunoconjugate described herein specifically binds to PD-1.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural environment. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

“Isolated polynucleotide (or nucleic acid) encoding [e.g. an immunoconjugate of the invention]” refers to one or more polynucleotide molecules encoding antibody heavy and light chains and/or IL-7 polypeptides (or fragments thereof), including such polynucleotide molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette comprises polynucleotide sequences that encode immunoconjugates of the invention or fragments thereof.

The term “vector” or “expression vector” refers to a DNA molecule that is used to introduce and direct the expression of a specific gene to which it is operably associated in a cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the cell, the ribonucleic acid molecule or protein that is encoded by the gene is produced by the cellular transcription and/or translation machinery. In one embodiment, the expression vector of the invention comprises an expression cassette that comprises polynucleotide sequences that encode immunoconjugates of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the immunoconjugates of the present invention. Host cells include cultured cells, e.g. mammalian cultured cells, such as HEK cells, CHO cells, BHK cells, NSO cells, SP2/0 cells, YO myeloma cells, P3X63 mouse myeloma cells, PER cells, PER. C6 cells or hybridoma cells, yeast cells, insect cells, and plant cells, to name only a few, but also cells comprised within a transgenic animal, transgenic plant or cultured plant or animal tissue.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen binding activity.

The term “monoclonal antibody” as used herein refers to an antibody obtained from a population of substantially homogeneous antibodies, i.e. the antibodies forming this population are essentially identical, except for possible post-translational modifications arising e.g. during manufacturing and/or storage. These antibodies are directed against the same epitope (or the same group of epitopes in the case of multispecific monoclonal antibodies, e.g. the same pair of epitopes in the case of bispecific monoclonal antibodies). This definition expressly excludes polyclonal antibody preparations which are mixtures of antibodies directed against different epitopes. Monoclonal antibodies in accordance with the present invention may be made by a variety of techniques, including but not limited to hybridoma methodology, recombinant DNA methods, phage-display methods, and methods utilizing transgenic animals containing all or part of the human immunoglobulin loci, such methods and other exemplary methods for making monoclonal antibodies being described herein. An “isolated” antibody is one which has been separated from a component of its natural environment, i.e. that is not in its natural milieu. No particular level of purification is required. For example, an isolated antibody can be removed from its native or natural environment. Recombinantly produced antibodies expressed in host cells are considered isolated for the purpose of the invention, as are native or recombinant antibodies which have been separated, fractionated, or partially or substantially purified by any suitable technique. As such, the immunoconjugates of the present invention are isolated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

The term “full-length antibody” refers to an antibody having the structure of an immunoglobulin comprising two light chains and two heavy chains, and comprising an Fc region as defined herein. In one aspect, the antibody is a full-length IgG1 antibody. An “antibody fragment” refers to a molecule other than a full-length antibody that comprises a portion of a full-length antibody that binds the antigen to which the full-length antibody binds. Examples of antibody fragments include but are not limited to Fv molecules, Fab molecules, Fab′ molecules, Fab′-SH molecules, F(ab′)2 molecules, diabodies, linear antibody molecules, single-chain antibody molecules (e.g., scFv and scFab molecules), and multispecific (e.g. bispecific) antibodies formed from antibody fragments.

A “diabody” is an antibody fragment with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993).

A “single chain Fab fragment” or “scFab” molecule is a polypeptide consisting of an antibody heavy chain variable domain (VH), an antibody heavy chain constant domain 1 (CH1), an antibody light chain variable domain (VL), an antibody light chain constant domain (CL) and a linker, wherein the antibody domains and the linker have one of the following orders in N-terminal to C-terminal direction: a) VH-CHI-linker-VL-CL, b) VL-CL-linker-VH-CH1, c) VH-CL-linker-VL-CH1 or d) VL-CHI-linker-VH-CL. In particular, said linker is a polypeptide of at least 30 amino acids, preferably between 32 and 50 amino acids. The single chain Fab fragments are stabilized via the natural disulfide bond between the CL domain and the CHI domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

A “single-chain variable fragment” or “scFv” molecule is a fusion protein of the variable domains of the heavy (VH) and light chain (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker.

A “multispecific antibody” is one having binding specificities for at least two different epitopes, i.e., different epitopes on different antigens or different epitopes on the same antigen. In one aspect, a multispecific antibody is a “bispecific antibody” having binding specificities for two epitopes. In certain aspects, the multispecific antibody has three or more binding specificities. In certain aspects, one of the binding specificities is for PD-1 and the other specificity/ies is for any other antigen(s). In certain aspects, multispecific antibodies may bind to two (or more) different epitopes of PD-1. Multispecific antibodies (e.g. bispecific antibodies) may comprise antibody fragments and/or one or more Fc region. The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “complementarity determining region” or “CDR” as used herein refers to each of the regions of an antibody variable domain which are hypervariable in sequence and which determine antigen binding specificity. Generally, antibodies comprise six CDRs: three in the VH (CDR-H1, CDR-H2, CDR-H3), and three in the VL (CDR-L1, CDR-L2, CDR-L3). CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis: http://www.abysis.org/abysis/sequence_input/key_annotation/key_annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (H2), and 96-101 (H3), according to Chothia and Lesk, J. Mol. Biol. 196:901-917 (1987) (“Chothia definition”);
(b) CDRs occurring at amino acid residues 24-34 (L1), 50-56 (L2), 89-97 (L3), 31-35B (H1), 50-65 (H2), and 95-102 (H3), according to Kabat 1991 (“Kabat definition”);
(c) antigen contacts occurring at amino acid residues 30-36 (L1), 46-55 (L2), 89-96 (L3), 30-35 (H1), 47-58 (H2), and 93-101 (H3), according to MacCallum et al. J. Mol. Biol. 262:732-745 (1996) (“Contact definition”); and
(d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27:55-77 (2003) (“IMGT definition”).

Unless otherwise indicated, the CDRs are determined herein according to Kabat 1991. One of skill in the art will understand that the CDR designations can also be determined according to Chothia, MacCallum, Lefranc, or any other scientifically accepted definition/system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

A “humanized” antibody refers to an antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The “class” of an antibody refers to the type of constant domain or constant region possessed by 5 its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain are glycine (G446) and lysine (K447, Kabat EU numbering). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. The corresponding sequence including a C-terminal glycine residue, or including a C-terminal glycine-lysine dipeptide, is also encompassed, however. Accordingly, in one aspect, a heavy chain including an Fc region as specified herein comprises an additional C-terminal glycine-lysine dipeptide (G446 and K447, Kabat EU numbering). In one aspect, a heavy chain including an Fc region as specified herein comprises an additional C-terminal glycine residue (G446, Kabat EU numbering). Unless otherwise specified herein, numbering of amino acid residues in the Fc region or heavy chain constant region is according to the EU numbering system, also called the EU index, as described in Kabat 1991.

The term “effector functions” when used in reference to antibodies refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

“Reduced binding”, for example reduced binding to an Fc receptor or CD25, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

As used herein, the term “immunoconjugate” refers to a polypeptide molecule that includes at least one IL-7 molecule and at least one antibody. The IL-7 molecule can be joined to the antibody by a variety of interactions and in a variety of configurations as described herein. In particular embodiments, the IL-7 molecule is fused to the antibody via a peptide linker. Particular immunoconjugates according to the invention essentially consist of one IL-7 molecule and an antibody joined by one or more linker sequences.

By “fused” is meant that the components (e.g. an antibody and an IL-7 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, immunoconjugates of the invention are used to delay development of a disease or to slow the progression of a disease.

### DETAILED DESCRIPTION OF THE EMBODIMENTS

### Mutant IL-7 Polypeptide

The IL-7 variants according to the present invention have advantageous properties for immunotherapy.

The mutant interleukin-7 (IL-7) polypeptide according to the invention comprises at least one amino acid mutation that reduces affinity of the mutant IL-7 polypeptide to the α-subunit of the IL-7 receptor and/or the IL-2Rγ subunit. In some embodiments, the mutant IL-7 polypeptide has a reduced affinity to the α-subunit of the IL-7 receptor and/or the IL-2Rγ subunit as compared to a reference IL-7 polypeptide which is identical to the mutant IL-7 except for the one or more mutations of the mutant IL-7 polypeptide. In some embodiments, the reference IL-7 polypeptide has amino acid residues of the wild-type form, e.g., of SEQ ID NO: 64, in those positions where the mutant IL-7 polypeptide has substitution(s), e.g., E at position 13 and G at position 85 for a mutant IL-7 polypeptide comprising K at position 13 and E at position 85 (numbering relative to the human IL-7 sequence SEQ ID NO: 64). In some embodiments, the mutant IL-7 polypeptide has a reduced affinity to the α-subunit of the IL-7 receptor and/or the IL-2Rγ subunit as compared to an interleukin-7 polypeptide comprising or consisting of the sequence of SEQ ID NO: 64.

Mutants of human IL-7 (hIL-7) with decreased affinity to IL-7Rα and/or IL-2Rγ may for example be generated by amino acid substitution at amino acid position 13 or 85 or combinations thereof (numbering relative to the human IL-7 sequence SEQ ID NO: 64). Exemplary amino acid substitutions include E13K and G85E. In one embodiment the mutant interleukin-7 (IL-7) polypeptide comprises amino acid substitutions at positions E13 and G85 of human IL-7 according to SEQ ID NO: 64. In one embodiment, the mutant interleukin-7 (IL-7) polypeptide comprises K at position 13 and E at position 85 according to SEQ ID NO: 64. In one embodiment of the invention the mutant interleukin-7 (IL-7) polypeptide comprises the amino acid substitutions E13K and G85E according to SEQ ID NO: 64. In some embodiments of the invention the mutant interleukin-7 polypeptide comprises an amino acid sequence of SEQ ID NO: 66. This interleukin-7 polypeptide exhibit substantially reduced affinity to the interleukin 7 receptor compared to a wild-type form of the IL-7 mutant. In some embodiments, the mutant IL-7 polypeptide comprises K at position 13 and E at position 85 (numbering relative to the human IL-7 sequence SEQ ID NO: 64) and an amino acid sequence that is at least about 70%, at least about 75%, at least about 80%, at least about 85%, at least about 90%, at least about 91%, at least about 92%, at least about 93%, at least about 94%, at least about 95%, at least about 96% or 100% identical to SEQ ID NO: 66. In some embodiments, the mutant interleukin-7 polypeptide comprises the amino acid substitutions E13K and G85E, wherein the numbering is relative to the human IL-7 sequence of SEQ ID NO: 64, and one or more further mutation as compared to the human IL-7 sequence of SEQ ID NO: 64.

Other exemplary amino acid substitutions include D74K and G85K. In one embodiment the mutant interleukin-7 (IL-7) polypeptide comprises amino acid substitutions at positions D74 and G85 of human IL-7 according to SEQ ID NO: 64. In one embodiment, the mutant interleukin-7 (IL-7) polypeptide comprises K at position 74 and K at position 85 according to SEQ ID NO: 64. In one embodiment of the invention the mutant interleukin-7 (IL-7) polypeptide comprises the amino acid substitutions D74K and G85K according to SEQ ID NO: 64. In some embodiments of the invention the mutant interleukin-7 polypeptide comprises an amino acid sequence of SEQ ID NO: 65. This interleukin-7 polypeptide exhibit substantially reduced affinity to the interleukin 7 receptor compared to a wild-type form of the IL-7 mutant. In some embodiments, the mutant IL-7 polypeptide comprises K at position 74 and K at position 85 (numbering relative to the human IL-7 sequence SEQ ID NO: 64) and an amino acid sequence that is at least about 70%, at least about 75%, at least about 80%, at least about 85%, at least about 90%, at least about 91%, at least about 92%, at least about 93%, at least about 94%, at least about 95%, at least about 96% or 100% identical to SEQ ID NO: 65. In some embodiments, the mutant interleukin-7 polypeptide comprises the amino acid substitutions D74K and G85K, wherein the numbering is relative to the human IL-7 sequence of SEQ ID NO: 64, and one or more further mutation as compared to the human IL-7 sequence of SEQ ID NO: 64. In some embodiments, the IL-7 mutants as disclosed herein have reduced affinity to IL-7Rα to allow PD-1 mediated delivery of IL-7 in cis (on the same cell) on PD-1 expressing CD4 T cells, compared to wild-type IL-7 which is mainly delivered in trans (on cells in close proximity) when in a PD1-IL-7 immunoconjugate.

In certain embodiments, said amino acid mutation reduces the affinity of the mutant IL-7 polypeptide to the IL-Ra and/or the IL-2Rγ by at least 5-fold, specifically at least 10-fold, more specifically at least 25-fold. In some embodiments, the mutant interleukin-7 (IL-7) polypeptide comprises the amino acid substitutions E13K and G85E and has at least a 5-fold, at least a 10-fold or at least a 25-fold reduction in affinity to the IL-Ra and/or the IL-2Rγ as compared to a reference IL-7 polypeptide which is identical to the mutant IL-7 polypeptide except for having E at position 13 and G at position 85 (numbering relative to the human IL-7 sequence of SEQ ID NO: 64). In some embodiments, the mutant interleukin-7 (IL-7) polypeptide comprises the amino acid substitutions D74K and G85K and has at least a 5-fold, at least a 10-fold or at least a 25-fold reduction in affinity to the IL-Ra and/or the IL-2Rγ as compared to a reference IL-7 polypeptide which is identical to the mutant IL-7 polypeptide except for having D at position 74 and G at position 85 (numbering relative to the human IL-7 sequence of SEQ ID NO: 64). In some embodiments, the mutant interleukin-7 (IL-7) polypeptide comprises the amino acid substitutions E13K and G85E (numbering relative to the human IL-7 sequence of SEQ ID NO: 64) and has at least a 5-fold, at least a 10-fold or at least a 25-fold reduction in affinity to the IL-Ra and/or the IL-2Rγ as compared to a reference IL-7 polypeptide comprising or consisting of the sequence of SEQ ID NO: 64. In some embodiments, the mutant interleukin-7 (IL-7) polypeptide comprises the amino acid substitutions D74K and G85K (numbering relative to the human IL-7 sequence of SEQ ID NO: 64) and has at least a 5-fold, at least a 10-fold or at least a 25-fold reduction in affinity to the IL-Rα and/or the IL-2Rγ as compared to a reference IL-7 polypeptide comprising or consisting of the sequence of SEQ ID NO: 64.

In a specific embodiment, the mutant IL-7 polypeptide can still elicit one or more of the cellular responses selected from the group consisting of: proliferation in T lymphocyte cells, effector functions in an primed T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment, the mutant IL-7 polypeptide comprises no more than 12, no more than 11, no more than 10, no more than 9, no more than 8, no more than 7, no more than 6, or no more than 5 amino acid mutations as compared to the corresponding wild-type IL-7 sequence, e.g. the human IL-7 sequence of SEQ ID NO: 64. In a particular embodiment, the mutant IL-7 polypeptide comprises no more than 5 amino acid mutations as compared to the corresponding wild-type IL-7 sequence, e.g. the human IL-7 sequence of SEQ ID NO: 64. In some embodiments, the mutant IL-7 polypeptide comprises an amino acid sequence that is at least about 70%, at least about 75%, at least about 80%, at least about 85%, at least about 90%, at least about 91%, at least about 92%, at least about 93%, at least about 94%, at least about 95% or at least about 96% identical to SEQ ID NO: 64.

### Immunoconjugates

Provided herein is also a protein comprising any of the mutant IL-7 polypeptides described herein, such as an immunoconjugate.

Immunoconjugates as described herein comprise an IL-7 molecule and an antibody. Such immunoconjugates significantly increase the efficacy of IL-7 therapy by directly targeting IL-7 e.g. into a tumor microenvironment. According to the invention, an antibody comprised in the immunoconjugate can be a whole antibody or immunoglobulin, or a portion or variant thereof that has a biological function such as antigen specific binding affinity.

The general benefits of immunoconjugate therapy are readily apparent. For example, an antibody comprised in an immunoconjugate may recognize a tumor-specific epitope and results in targeting of the immunoconjugate molecule to the tumor site. Therefore, high concentrations of IL-7 can be delivered into the tumor microenvironment, thereby resulting in activation and proliferation of a variety of immune effector cells mentioned herein using a much lower dose of the immunoconjugate than would be required for unconjugated IL-7. However, this characteristic of IL-7 immunoconjugates may again aggravate potential side effects of the IL-7 molecule: Because of the significantly longer circulating half-life of IL-7 immunoconjugate in the bloodstream relative to unconjugated IL-7, the probability for IL-7 or other portions of the fusion protein molecule to activate components generally present in the vasculature is increased. The same concern applies to other fusion proteins that contain IL-7 fused to another moiety such as Fc or albumin, resulting in an extended half-life of IL-7 in circulation. Therefore, immunoconjugates comprising a mutant IL-7 polypeptide as described herein with reduced toxicity compared to wild-type forms of IL-7, is particularly advantageous.

As described hereinabove, targeting IL-7 directly to immune effector cells rather than tumor cells may be advantageous for IL-7 immunotherapy.

Accordingly, the invention provides a mutant IL-7 polypeptide as described hereinbefore, and an antibody that binds to PD-1. In one embodiment the mutant IL-7 polypeptide and the antibody form a fusion protein, i.e. the mutant IL-7 polypeptide shares a peptide bond with the antibody. In some embodiments, the antibody comprises an Fc domain composed of a first and a second subunit. In a specific embodiment the mutant IL-7 polypeptide is fused at its amino-terminal amino acid to the carboxy-terminal amino acid of one of the subunits of the Fc domain, optionally through a linker peptide. In some embodiments, the antibody is a full-length antibody. In some embodiments, the antibody is an immunoglobulin molecule, particularly an IgG class immunoglobulin molecule, more particularly an IgG₁ subclass immunoglobulin molecule. In one such embodiment, the mutant IL-7 polypeptide shares an amino-terminal peptide bond with one of the immunoglobulin heavy chains. In certain embodiments the antibody is an antibody fragment. In some embodiments the antibody is a Fab molecule or a scFv molecule. In one embodiment the antibody is a Fab molecule. In another embodiment the antibody is a scFv molecule. The immunoconjugate may also comprise more than one antibody. Where more than one antibody is comprised in the immunoconjugate, e.g. a first and a second antibody, each antibody can be independently selected from various forms of antibodies and antibody fragments. For example, the first antibody can be a Fab molecule and the second antibody can be a scFv molecule. In a specific embodiment each of said first and said second antibodies is a scFv molecule or each of said first and said second antibodies is a Fab molecule. In a particular embodiment each of said first and said second antibodies is a Fab molecule. In one embodiment each of said first and said second antibodies binds to PD-1.

### Immunoconjugate Formats

Exemplary immunoconjugate formats are described in PCT publication no. WO 2011/020783, which is incorporated herein by reference in its entirety. These immunoconjugates comprise at least two antibodies. Thus, in one embodiment, the immunoconjugate according to the present invention comprises a mutant IL-7 polypeptide as described herein, and at least a first and a second antibody. In a particular embodiment, said first and second antibody are independently selected from the group consisting of an Fv molecule, particularly a scFv molecule, and a Fab molecule. In a specific embodiment, said mutant IL-7 polypeptide shares an amino- or carboxy-terminal peptide bond with said first antibody and said second antibody shares an amino- or carboxy-terminal peptide bond with either i) the mutant IL-7 polypeptide or ii) the first antibody. In a particular embodiment, the immunoconjugate consists essentially of a mutant IL-7 polypeptide and first and second antibodies, particularly Fab molecules, joined by one or more linker sequences. Such formats have the advantage that they bind with high affinity to the target antigen (PD-1), but provide only monomeric binding to the IL-7 receptor, thus avoiding targeting the immunoconjugate to IL-7 receptor bearing immune cells at other locations than the target site. In a particular embodiment, a mutant IL-7 polypeptide shares a carboxy-terminal peptide bond with a first antibody, particularly a first Fab molecule, and further shares an amino-terminal peptide bond with a second antibody, particularly a second Fab molecule. In another embodiment, a first antibody, particularly a first Fab molecule, shares a carboxy-terminal peptide bond with a mutant IL-7 polypeptide, and further shares an amino-terminal peptide bond with a second antibody, particularly a second Fab molecule. In another embodiment, a first antibody, particularly a first Fab molecule, shares an amino-terminal peptide bond with a first mutant IL-7 polypeptide, and further shares a carboxy-terminal peptide with a second antibody, particularly a second Fab molecule. In a particular embodiment, a mutant IL-7 polypeptide shares a carboxy-terminal peptide bond with a first heavy chain variable region and further shares an amino-terminal peptide bond with a second heavy chain variable region. In another embodiment a mutant IL-7 polypeptide shares a carboxy-terminal peptide bond with a first light chain variable region and further shares an amino-terminal peptide bond with a second light chain variable region. In another embodiment, a first heavy or light chain variable region is joined by a carboxy-terminal peptide bond to a mutant IL-7 polypeptide and is further joined by an amino-terminal peptide bond to a second heavy or light chain variable region. In another embodiment, a first heavy or light chain variable region is joined by an amino-terminal peptide bond to a mutant IL-7 polypeptide and is further joined by a carboxy-terminal peptide bond to a second heavy or light chain variable region. In one embodiment, a mutant IL-7 polypeptide shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a mutant IL-7 polypeptide and further shares an amino-terminal peptide bond with a second Fab heavy or light chain. In other embodiments, a first Fab heavy or light chain shares an amino-terminal peptide bond with a mutant IL-7 polypeptide and further shares a carboxy-terminal peptide bond with a second Fab heavy or light chain. In one embodiment, the immunoconjugate comprises a mutant IL-7 polypeptide sharing an amino-terminal peptide bond with one or more scFv molecules and further sharing a carboxy-terminal peptide bond with one or more scFv molecules.

Particularly suitable formats for the immunoconjugates according to the present invention, however comprise an immunoglobulin molecule as antibody. Such immunoconjugate formats are described in WO 2012/146628, which is incorporated herein by reference in its entirety.

Accordingly, in particular embodiments, the immunoconjugate comprises a mutant IL-7 polypeptide as described herein and an immunoglobulin molecule that binds to PD-1, particularly an IgG molecule, more particularly an IgG₁ molecule. In one embodiment the immunoconjugate comprises not more than one mutant IL-7 polypeptide. In one embodiment the immunoconjugate comprises not more than one IL-7 polypeptide. In one embodiment the immunoconjugate comprises only one antibody, wherein the antibody is an immunoglobulin. In one embodiment the immunoconjugate comprises only one IL-7 polypeptide and only one antibody, wherein the antibody is an immunoglobulin. In one embodiment the immunoglobulin molecule is human. In one embodiment, the immunoglobulin molecule comprises a human constant region, e.g. a human CH1, CH2, CH3 and/or CL domain. In one embodiment, the immunoglobulin comprises a human Fc domain, particularly a human IgG₁ Fc domain. In one embodiment the mutant IL-7 polypeptide shares an amino- or carboxy-terminal peptide bond with the immunoglobulin molecule. In one embodiment, the immunoconjugate essentially consists of a mutant IL-7 polypeptide and an immunoglobulin molecule, particularly an IgG molecule, more particularly an IgG₁ molecule, joined by one or more linker sequences. In a specific embodiment the mutant IL-7 polypeptide is fused at its amino-terminal amino acid to the carboxy-terminal amino acid of one of the immunoglobulin heavy chains, optionally through a linker peptide.

The mutant IL-7 polypeptide may be fused to the antibody directly or through a linker peptide, comprising one or more amino acids, typically about 2-20 amino acids. Linker peptides are known in the art and are described herein. Suitable, non-immunogenic linker peptides include, for example, (G₄S)ₙ, (SG₄)ₙ, (G₄S)ₙ or G₄(SG₄)ₙ linker peptides. “n” is generally an integer from 1 to 10, typically from 2 to 4. In one embodiment the linker peptide has a length of at least 5 amino acids, in one embodiment a length of 5 to 100, in a further embodiment of 10 to 50 amino acids. In a particular embodiment, the linker peptide has a length of 15 amino acids. In one embodiment the linker peptide is (GxS)ₙ or (GxS)ₙGₘ with G-glycine, S-serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=3, in a further embodiment x=4, n=2 and m=4. In a particular embodiment the linker peptide is (G₄S)₂G₄. In one embodiment, the linker peptide has (or consists of) the amino acid sequence of (G₄S)₂G₄. In a preferred embodiment, the linker peptide is (G₂SG₂)₃ (SEQ ID NO: 75). In one embodiment, the linker peptide has (or consists of) the amino acid sequence of SEQ ID NO: 75.

In a particular embodiment, the immunoconjugate comprises a mutant IL-7 molecule and an immunoglobulin molecule, particularly an IgG₁ subclass immunoglobulin molecule, that binds to PD-1, wherein the mutant IL-7 molecule is fused at its amino-terminal amino acid to the carboxy-terminal amino acid of one of the immunoglobulin heavy chains through the linker peptide of SEQ ID NO: 75.

In a particular embodiment, the immunoconjugate comprises a mutant IL-7 molecule and an antibody that binds to PD-1, wherein the antibody comprises an Fc domain, particularly a human IgG₁ Fc domain, composed of a first and a second subunit, and the mutant IL-7 molecule is fused at its amino-terminal amino acid to the carboxy-terminal amino acid of one of the subunits of the Fc domain through the linker peptide of SEQ ID NO: 75.

### PD-1 Antibodies

Preferably, the antibody comprised in the immunoconjugate of the invention binds to PD-1, particularly human PD-1, and is able to direct the mutant IL-7 polypeptide to a target site where PD-1 is expressed, particularly to a T cell that expresses PD-1, for example associated with a tumor.

Suitable PD-1 antibodies that may be used in the immunoconjugate of the invention are described in WO 2017/055443 A1, which is incorporated herein by reference in its entirety.

The immunoconjugate of the invention may comprise two or more antibodies, which may bind to the same or to different antigens. In particular embodiments, however, each of these antibodies binds to PD-1. In one embodiment, the antibody comprised in the immunoconjugate of the invention is monospecific. In a particular embodiment, the immunoconjugate comprises a single, monospecific antibody, particularly a monospecific immunoglobulin molecule.

The antibody can be any type of antibody or fragment thereof that retains specific binding to PD-1, particularly human PD-1. Antibody fragments include, but are not limited to, Fv molecules, scFv molecule, Fab molecule, and F(ab′)₂ molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG₁ subclass immunoglobulin.

In some embodiments, the antibody is a monoclonal antibody.

In some embodiments, the antibody comprises a CDR-H1 comprising the amino acid sequence of SEQ ID NO:67, a CDR-H2 comprising the amino acid sequence of SEQ ID NO:68, a CDR-H3 comprising the amino acid sequence of SEQ ID NO:69, a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72. In some embodiments, the heavy and/or light chain variable region is a humanized variable region. In some embodiments, the heavy and/or light chain variable region comprises human framework regions (FR).

In some embodiments, the antibody comprises a heavy chain variable region (VH) comprising an amino acid sequence that is at least about 95%, 96%, 97%, 98%, 99% or 100% identical to the amino acid sequence of SEQ ID NO:73. In some embodiments, the antibody comprises a light chain variable region (VL) comprising an amino acid sequence that is at least about 95%, 96%, 97%, 98%, 99% or 100% identical to an amino acid sequence of SEQ ID NO:74. In a particular embodiment, the antibody comprises (a) a heavy chain variable region (VH) comprising the amino acid sequence of SEQ ID NO: 73, and (b) a light chain variable region (VL) comprising the amino acid sequence of SEQ ID NO: 74.

In some embodiments, the antibody is a humanized antibody. In one embodiment, the antibody is an immunoglobulin molecule comprising a human constant region, particularly an IgG class immunoglobulin molecule comprising a human CH1, CH2, CH3 and/or CL domain.

### Fc Domain

In particular embodiments, the antibody comprised in the immunoconjugates according to the invention comprises an Fc domain, composed of a first and a second subunit. The Fc domain of an antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the immunoconjugate of the invention comprises not more than one Fc domain. In one embodiment the Fc domain of the antibody comprised in the immunoconjugate is an IgG Fc domain. In a particular embodiment the Fc domain is an IgG Fc domain. In another embodiment the Fc domain is an IgG₄ Fc domain. In a more specific embodiment, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is a human Fc domain. In an even more particular embodiment, the Fc domain is a human IgG₁ Fc domain.

### Fc Domain Modifications Promoting Heterodimerization

Immunoconjugates according to the invention comprise a mutant IL-7 polypeptide, particularly a single (not more than one) mutant IL-7 polypeptide, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the immunoconjugate in recombinant production, it will thus be advantageous to introduce in the Fc domain of the antibody a modification promoting the association of the desired polypeptides.

Accordingly, in particular embodiments, the Fc domain of the antibody comprised in the immunoconjugate according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed).

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the antibody comprised in the immunoconjugate an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine(S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain (the “knobs” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some embodiments, the second subunit of the Fc domain additionally comprises the amino acid substitutions H435R and Y436F (numbering according to Kabat EU index).

In a particular embodiment the mutant IL-7 polypeptide is fused (optionally through a linker peptide) to the first subunit of the Fc domain (comprising the “knob” modification). Without wishing to be bound by theory, fusion of the mutant IL-7 polypeptide to the knob-containing subunit of the Fc domain will (further) minimize the generation of immunoconjugates comprising two mutant IL-7 polypeptides (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment the heterodimerization approach described in EP 1870459, is used alternatively. This approach is based on the introduction of charged amino acids with opposite charges at specific amino acid positions in the CH3/CH3 domain interface between the two subunits of the Fc domain. One preferred embodiment for the antibody comprised in the immunoconjugate of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another embodiment, the antibody comprised in the immunoconjugate of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another embodiment, the antibody comprised in the immunoconjugate of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment, the heterodimerization approach described in WO 2013/157953 is used alternatively. In one embodiment, a first CH3 domain comprises amino acid mutation T366K and a second CH3 domain comprises amino acid mutation L351D (numberings according to Kabat EU index). In a further embodiment, the first CH3 domain comprises further amino acid mutation L351K. In a further embodiment, the second CH3 domain comprises further an amino acid mutation selected from Y349E, Y349D and L368E (preferably L368E) (numberings according to Kabat EU index).

In one embodiment, the heterodimerization approach described in WO 2012/058768 is used alternatively. In one embodiment, a first CH3 domain comprises amino acid mutations L351Y, Y407A and a second CH3 domain comprises amino acid mutations T366A, K409F. In a further embodiment, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index). In a further embodiment, a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment, the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A. In one embodiment, a first CH3 domain comprises amino acid mutation T366Y and a second CH3 domain comprises amino acid mutation Y407T (numberings according to Kabat EU index).

In one embodiment, the antibody comprised in the immunoconjugate or its Fc domain is of IgG₂ subclass and the heterodimerization approach described in WO 2010/129304 is used alternatively.

In an alternative embodiment, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment, the first CH3 domain further comprises amino acid substitution of K409 or R409 with a negatively charged amino acid (e.g. glutamic acid (E), or aspartic acid (D), preferably K409D or R409D). In a further embodiment, the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment, the heterodimerization approach described in WO 2007/147901 is used alternatively. In one embodiment, a first CH3 domain comprises amino acid mutations K253E, D282K, and K322D and a second CH3 domain comprises amino acid mutations D239K, E240K, and K292D (numberings according to Kabat EU index).

In still another embodiment, the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the immunoconjugate favorable pharmacokinetic properties, including a long serum half-life which contributes to good accumulation in the target tissue and a favorable tissue-blood distribution ratio. At the same time it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-7 polypeptide and the long half-life of the immunoconjugate, results in excessive activation of cytokine receptors and severe side effects upon systemic administration. Accordingly, in particular embodiments, the Fc domain of the antibody comprised in the immunoconjugate according to the invention exhibits reduced binding affinity to an Fc receptor and/or reduced effector function, as compared to a native IgG₁ Fc domain. In one such embodiment the Fc domain (or the antibody comprising said Fc domain) exhibits less than 50%, preferably less than 20%, more preferably less than 10% and most preferably less than 5% of the binding affinity to an Fc receptor, as compared to a native IgG₁ Fc domain (or an antibody comprising a native IgG₁ Fc domain), and/or less than 50%, preferably less than 20%, more preferably less than 10% and most preferably less than 5% of the effector function, as compared to a native IgG₁ Fc domain domain (or an antibody comprising a native IgG₁ Fc domain). In one embodiment, the Fc domain domain (or an antibody comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or an antibody comprising said Fc domain) exhibits greater than about 70%, particularly greater than about 80%, more particularly greater than about 90% of the binding affinity of a native IgG₁ Fc domain (or an antibody comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the antibody comprised in the immunoconjugate comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the antibody comprising an engineered Fc domain exhibits less than 20%, particularly less than 10%, more particularly less than 5% of the binding affinity to an Fc receptor as compared to an antibody comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or an antibody comprising said Fc domain) exhibits greater than about 70% of the binding affinity of a non-engineered form of the Fc domain (or an antibody comprising said non-engineered form of the Fc domain) to FcRn. The Fc domain, or antibody comprised in the immunoconjugate of the invention comprising said Fc domain, may exhibit greater than about 80% and even greater than about 90% of such affinity. In certain embodiments the Fc domain of the antibody comprised in the immunoconjugate is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or an antibody comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329 (numberings according to Kabat EU index). In a more specific embodiment the Fc domain comprises an amino acid substitution at a position selected from the group of L234, L235 and P329 (numberings according to Kabat EU index). In some embodiments the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G (numberings according to Kabat EU index). In one embodiment the Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the Fc domain comprises amino acid substitutions at positions P329, L234 and L235 (numberings according to Kabat EU index). In more particular embodiments the Fc domain comprises the amino acid mutations L234A, L235A and P329G (“P329G LALA”, “PGLALA” or “LALAPG”). Specifically, in particular embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the antibody comprised in the immunoconjugate of the invention is an IgG₄ Fc domain, particularly a human IgG₄ Fc domain. In one embodiment the IgG₄ Fc domain comprises amino acid substitutions at position S228, specifically the amino acid substitution S228P (numberings according to Kabat EU index). To further reduce its binding affinity to an Fc receptor and/or its effector function, in one embodiment the IgG₄ Fc domain comprises an amino acid substitution at position L235, specifically the amino acid substitution L235E (numberings according to Kabat EU index). In another embodiment, the IgG₄ Fc domain comprises an amino acid substitution at position P329, specifically the amino acid substitution P329G (numberings according to Kabat EU index). In a particular embodiment, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing FcγIIIa receptor.

Effector function of an Fc domain, or an antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the Fc domain, or antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18 (12): 1759-1769 (2006); WO 2013/120929).

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions E13K and G85E (numbering relative to the human IL-7 sequence SEQ ID NO: 64); and wherein the antibody comprises (a) a heavy chain variable region (VH) comprising the amino acid sequence of SEQ ID NO:73, and (b) a light chain variable region (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions D74K and G85K (numbering relative to the human IL-7 sequence SEQ ID NO: 64); and wherein the antibody comprises (a) a heavy chain variable region (VH) comprising the amino acid sequence of SEQ ID NO:73, and (b) a light chain variable region (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 66, and wherein the antibody comprises (a) a heavy chain variable region (VH) comprising the amino acid sequence of SEQ ID NO:73, and (b) a light chain variable region (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 65, and wherein the antibody comprises (a) a heavy chain variable region (VH) comprising the amino acid sequence of SEQ ID NO:73, and (b) a light chain variable region (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 65, and wherein the antibody comprises (a) two heavy chain variable regions (VH), each comprising the amino acid sequence of SEQ ID NO:73, and (b) two light chain variable regions (VL), each comprising the amino acid sequence of SEQ ID NO:74. In one aspect, the invention provides an immunoconjugate comprising a mutant IL-7 polypeptide and an antibody that binds to PD-1, wherein the mutant IL-7 polypeptide comprises the amino acid sequence of SEQ ID NO: 66, and wherein the antibody comprises (a) two heavy chain variable regions (VH), each comprising the amino acid sequence of SEQ ID NO:73, and (b) two light chain variable regions (VL), each comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions E13K and G85E (numbering relative to the human IL-7 sequence SEQ ID NO: 64); and wherein the antibody comprises (a) a heavy chain variable region (VH) comprising the amino acid sequence of SEQ ID NO:73, and (b) a light chain variable region (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions D74K and G85K (numbering relative to the human IL-7 sequence SEQ ID NO: 64); and wherein the antibody comprises (a) a heavy chain variable region (VH) comprising the amino acid sequence of SEQ ID NO:73, and (b) a light chain variable regions (VL) comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions E13K and G85E (numbering relative to the human IL-7 sequence SEQ ID NO: 64); and wherein the antibody comprises (a) two heavy chain variable regions (VH), each comprising the amino acid sequence of SEQ ID NO:73, and (b) two light chain variable regions (VL), each comprising the amino acid sequence of SEQ ID NO:74.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions D74K and G85K (numbering relative to the human IL-7 sequence SEQ ID NO: 64); and wherein the antibody comprises (a) two heavy chain variable regions (VH), each comprising the amino acid sequence of SEQ ID NO:73, and (b) two light chain variable regions (VL), each comprising the amino acid sequence of SEQ ID NO:74. In one embodiment according to any of the above aspects of the invention, the antibody is an IgG class immunoglobulin, comprising a human IgG₁ Fc domain composed of a first and a second subunit, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-7 polypeptide may be fused at its amino-terminal amino acid to the carboxy-terminal amino acid of the first subunit of the Fc domain, through a linker peptide of SEQ ID NO: 75.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:53, a polypeptide comprising an amino acid sequence that is at least about 80%, 85%, 90%, 95%, 96%, 97%, 98%, 99% or 100% identical to the sequence of SEQ ID NO:54, and a polypeptide comprising an amino acid sequence that is at least about 80%, 85%, 90%, 95%, 96%, 97%, 98%, 99% or 100% identical to the sequence of SEQ ID NO:57. Preferably, in the aforementioned embodiment, the percentage of sequence identity to the reference sequence is the same for all three polypeptides, e.g., 90% of sequence identity to SEQ ID NOs: 53, 54 and 57, respectively.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:53, a polypeptide comprising an amino acid sequence that is at least about 80%, 85%, 90%, 95%, 96%, 97%, 98%, 99% or 100% identical to the sequence of SEQ ID NO:54, and a polypeptide comprising an amino acid sequence that is at least about 80%, 85%, 90%, 95%, 96%, 97%, 98%, 99% or 100% identical to the sequence of SEQ ID NO:56. Preferably, in the aforementioned embodiment, the percentage of sequence identity to the reference sequence is the same for all three polypeptides, e.g., 90% of sequence identity to SEQ ID NOs: 53, 54 and 56, respectively.

In one aspect, the invention provides an immunoconjugate comprising a polypeptide comprising an amino acid sequence of SEQ ID NO:53, a polypeptide comprising an amino acid sequence SEQ ID NO: 54, and a polypeptide comprising an amino acid sequence of SEQ ID NO:57.

In one aspect, the invention provides an immunoconjugate comprising a polypeptide comprising an amino acid sequence of SEQ ID NO:53, a polypeptide comprising an amino acid sequence of SEQ ID NO:54, and a polypeptide comprising an amino acid sequence of SEQ ID NO:56.

In one aspect, the invention provides an immunoconjugate comprising two polypeptides, each comprising an amino acid sequence of SEQ ID NO:53, a polypeptide comprising an amino acid sequence SEQ ID NO:54, and a polypeptide comprising an amino acid sequence of SEQ ID NO:57.

In one aspect, the invention provides an immunoconjugate comprising two polypeptides, each comprising an amino acid sequence of SEQ ID NO:53, a polypeptide comprising an amino acid sequence of SEQ ID NO:54, and a polypeptide comprising an amino acid sequence of SEQ ID NO: 56.

In one aspect, the invention provides an immunoconjugate consisting of two polypeptides, each consisting of an amino acid sequence of SEQ ID NO:53, a polypeptide consisting of an amino acid sequence SEQ ID NO:54, and a polypeptide consisting of an amino acid sequence of SEQ ID NO: 57.

In one aspect, the invention provides an immunoconjugate consisting of two polypeptides, each consisting of an amino acid sequence of SEQ ID NO:53, a polypeptide consisting of an amino acid sequence of SEQ ID NO:54, and a polypeptide consisting of an amino acid sequence of SEQ ID NO: 56.

### Polynucleotides

The invention further provides isolated polynucleotides encoding any of the proteins disclosed herein, including the mutant IL-7 polypeptides, proteins comprising a mutant IL-7 polypeptide, such as immunoconjugates, or any parts or portions thereof.

The polynucleotides encoding any of the proteins, such as the immunoconjugates of the invention, may be expressed as a single polynucleotide that encodes the entire protein or as multiple (e.g., two or more) polynucleotides that are co-expressed. Polypeptides encoded by polynucleotides that are co-expressed may associate through, e.g., disulfide bonds or other means to form a functional protein, e.g., a functional immunoconjugate. For example, the light chain portion of an antibody may be encoded by a separate polynucleotide from the portion of the immunoconjugate comprising the heavy chain portion of the antibody and the mutant IL-7 polypeptide. When co-expressed, the heavy chain polypeptides will associate with the light chain polypeptides to form the immunoconjugate. In another example, the portion of the immunoconjugate comprising one of the two Fc domain subunits and the mutant IL-7 polypeptide could be encoded by a separate polynucleotide from the portion of the immunoconjugate comprising the other of the two Fc domain subunits. When co-expressed, the Fc domain subunits will associate to form the Fc domain.

In some embodiments, the isolated polynucleotide encodes the entire immunoconjugate according to the invention as described herein. In other embodiments, the isolated polynucleotide encodes a polypeptide comprised in the immunoconjugate according to the invention as described herein.

In one embodiment, an isolated polynucleotide of the invention encodes the heavy chain of the antibody comprised in the immunoconjugate (e.g. an immunoglobulin heavy chain), and the mutant IL-7 polypeptide. In another embodiment, an isolated polynucleotide of the invention encodes the light chain of the antibody comprised in the immunoconjugate. In another embodiment, an isolated polynucleotide of the invention encodes the heavy chain of the antibody comprised in the immunoconjugate without the mutant IL-7 polypeptide.

In certain embodiments, the polynucleotide or nucleic acid is DNA. In other embodiments, a polynucleotide of the present invention is RNA, for example, in the form of messenger RNA (mRNA). RNA of the present invention may be single stranded or double stranded.

Provided herein is also a vector comprising one or more of the polynucleotides described herein. Moreover, provided herein is also a host cell comprising one or more of the polynucleotides described herein, e.g., a host cell comprising one or more vectors comprising such one or more polynucleotides.

### Recombinant Methods

Mutant IL-7 polypeptides useful in the invention can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. The sequence of native human IL-7 is shown in SEQ ID NO: 64. Substitution or insertion may involve natural as well as non-natural amino acid residues. Amino acid modification includes well known methods of chemical modification such as the addition of glycosylation sites or carbohydrate attachments, and the like.

Proteins of the invention, e.g., immunoconjugates or other proteins comprising a mutant IL-7 as described herein, may be obtained, for example, by solid-state peptide synthesis (e.g. Merrifield solid phase synthesis) or recombinant production. For recombinant production, one or more polynucleotide encoding the protein, such as the immunoconjugate as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of the protein, e.g., an immunoconjugate or a part thereof, along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the protein, e.g., the immunoconjugate or a part thereof, (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the protein of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the protein may be included within or at the ends of the protein encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) one or more vector comprising one or more polynucleotide that encodes the protein of the invention, such as the immunoconjugate. As used herein, the term “host cell” refers to any kind of cellular system which can be engineered to generate the proteins of the invention, such as the immunoconjugates, or parts thereof. Host cells suitable for replicating and for supporting expression of proteins, such as the immunoconjugates, are well known in the art. Such cells may be transfected or transduced as appropriate with the particular expression vector and large quantities of vector containing cells can be grown for seeding large scale fermenters to obtain sufficient quantities of the protein, e.g., immunoconjugate, for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a mutant-IL-7 polypeptide fused to either the heavy or the light chain of an antibody may be engineered so as to also express the other of the antibody chains such that the expressed mutant IL-7 fusion product is an antibody that has both a heavy and a light chain.

In one embodiment, a method of producing a protein comprising a mutant IL-7 polypeptide according to the invention is provided, wherein the method comprises culturing a host cell comprising one or more polynucleotide encoding the protein, as provided herein, under conditions suitable for expression of the protein, and optionally recovering the protein from the host cell (or host cell culture medium).

In one embodiment, a method of producing an immunoconjugate according to the invention is provided, wherein the method comprises culturing a host cell comprising one or more polynucleotide encoding the immunoconjugate, as provided herein, under conditions suitable for expression of the immunoconjugate, and optionally recovering the immunoconjugate from the host cell (or host cell culture medium).

In the immunoconjugate of the invention, the mutant IL-7 polypeptide may be genetically fused to the antibody, or may be chemically conjugated to the antibody. Genetic fusion of the IL-7 polypeptide to the antibody can be designed such that the IL-7 sequence is fused directly to the polypeptide or indirectly through a linker sequence. The composition and length of the linker may be determined in accordance with methods well known in the art and may be tested for efficacy. Particular linker peptides are described herein. Additional sequences may also be included to incorporate a cleavage site to separate the individual components of the fusion if desired, for example an endopeptidase recognition sequence. In addition, an IL-7 fusion protein may also be synthesized chemically using methods of polypeptide synthesis as is well known in the art (e.g. Merrifield solid phase synthesis). Mutant IL-7 polypeptides may be chemically conjugated to other molecules, e.g. antibodies, using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-7 and can readily be identified by those skilled in the art. Alternatively, or in addition, mutant IL-7 and/or the molecule to which it is intended to be conjugated may be chemically derivatized such that the two can be conjugated in a separate reaction as is also well known in the art.

The immunoconjugates of the invention comprise an antibody. Methods to produce antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Immunoconjugates, antibodies, and methods for producing the same are also described in detail e.g. in PCT publication nos. WO 2011/020783, WO 2012/107417, and WO 2012/146628, each of which are incorporated herein by reference in their entirety.

Any animal species of antibody may be used in the immunoconjugates of the invention. Non-limiting antibodies useful in the present invention can be of murine, primate, or human origin. If the immunoconjugate is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human antibodies can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolation from human antibody libraries, as described herein.

Antibodies useful in the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936. Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Further chemical modification of any of the proteins described herein, e.g., the immunoconjugate, may be desirable. For example, problems of immunogenicity and short half-life may be improved by conjugation to substantially straight chain polymers such as polyethylene glycol (PEG) or polypropylene glycol (PPG) (see e.g. WO 87/00056).

A protein of the present disclosure prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immunoconjugate or other protein comprising the mutant IL-7 polypeptide binds. For example, an antibody which specifically binds the mutant IL-7 polypeptide may be used. For affinity chromatography purification of immunoconjugates or other proteins comprising the mutant IL-7 polypeptide of the invention, a matrix with protein A or protein G may be used. For example, sequential Protein A or G affinity chromatography and size exclusion chromatography can be used to isolate an immunoconjugate essentially as described in the Examples. The purity of the purified protein can be determined by any of a variety of well known analytical methods including gel electrophoresis, high pressure liquid chromatography, and the like.

### Compositions, Formulations, and Routes of Administration

In a further aspect, the invention provides pharmaceutical compositions comprising a protein as described herein, such as an immunoconjugate or any other protein comprising a mutant IL-7 polypeptide, e.g., for use in any of the below therapeutic methods. In one embodiment, a pharmaceutical composition comprises any of the proteins provided herein and a pharmaceutically acceptable carrier. In another embodiment, a pharmaceutical composition comprises any of the proteins provided herein and at least one additional therapeutic agent, e.g., as described below.

Further provided is a method of producing a protein of the invention in a form suitable for administration in vivo, the method comprising (a) obtaining a protein according to the invention, e.g., an immunoconjugate, and (b) formulating the protein with at least one pharmaceutically acceptable carrier, whereby a preparation of protein is formulated for administration in vivo.

Pharmaceutical compositions of the present invention comprise a therapeutically effective amount of the protein, e.g., immunoconjugate, dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains a protein described herein, e.g., an immunoconjugate, and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

A protein of the invention, e.g., an immunoconjugate, (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the protein of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the protein may be in powder form for constitution with a suitable vehicle, e.g., sterile pyrogen-free water, before use. Sterile injectable solutions are prepared by incorporating the protein of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the proteins described herein may also be formulated as a depot preparation. Such long acting formulations may be administered by implantation (for example subcutaneously or intramuscularly) or by intramuscular injection. Thus, for example, the protein may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the proteins of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The proteins described herein may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the mutant IL-7 polypeptides and proteins comprising a mutant IL-7 polypeptide, such as the immunoconjugates, provided herein may be used in therapeutic methods. The mutant IL-7 polypeptides and proteins comprising a mutant IL-7 polypeptide, such as the immunoconjugates, of the invention may be used as immunotherapeutic agents, for example in the treatment of cancers.

For use in therapeutic methods, mutant IL-7 polypeptides and proteins comprising a mutant IL-7 polypeptide, such as the immunoconjugates, of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Mutant IL-7 polypeptides and proteins comprising a mutant IL-7 polypeptide, such as the immunoconjugates, of the invention may be particularly useful in treating disease states where stimulation of the immune system of the host is beneficial, in particular conditions where an enhanced cellular immune response is desirable. These may include disease states where the host immune response is insufficient or deficient. Disease states for which the mutant IL-7 polypeptides and proteins comprising a mutant IL-7 polypeptide, such as the immunoconjugates, of the invention may be administered comprise, for example, a tumor or infection where a cellular immune response would be a critical mechanism for specific immunity. The mutant IL-7 polypeptides and proteins comprising a mutant IL-7 polypeptide, such as the immunoconjugates, of the invention may be administered per se or in any suitable pharmaceutical composition.

In one aspect, mutant IL-7 polypeptides and proteins comprising a mutant IL-7 polypeptide, such as the immunoconjugates, of the invention for use as a medicament are provided. In further aspects, mutant IL-7 polypeptides and proteins comprising a mutant IL-7 polypeptide, such as the immunoconjugates, of the invention for use in treating a disease are provided. In certain embodiments, mutant IL-7 polypeptides and proteins comprising a mutant IL-7 polypeptide, such as the immunoconjugates, of the invention for use in a method of treatment are provided. In one embodiment, the invention provides a protein comprising a mutant IL-7 polypeptide as described herein for use in the treatment of a disease in an individual in need thereof. In one embodiment, the invention provides an immunoconjugate as described herein for use in the treatment of a disease in an individual in need thereof. In one embodiment, the invention provides a mutant IL-7 polypeptide as described herein for use in the treatment of a disease in an individual in need thereof. In certain embodiments, the invention provides a protein comprising a mutant IL-7 polypeptide for use in a method of treating an individual having a disease comprising administering to the individual a therapeutically effective amount of the protein. In certain embodiments, the invention provides an immunoconjugate for use in a method of treating an individual having a disease comprising administering to the individual a therapeutically effective amount of the immunoconjugate. In certain embodiments, the invention provides a mutant IL-7 polypeptide for use in a method of treating an individual having a disease comprising administering to the individual a therapeutically effective amount of the mutant IL-7 polypeptide. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a protein comprising a mutant IL-7 polypeptide for use in stimulating the immune system. In certain embodiments, the invention provides a protein comprising a mutant IL-7 polypeptide for use in a method of stimulating the immune system in an individual comprising administering to the individual an effective amount of the protein to stimulate the immune system. In further embodiments, the invention provides an immunoconjugate for use in stimulating the immune system. In certain embodiments, the invention provides an immunoconjugate for use in a method of stimulating the immune system in an individual comprising administering to the individual an effective amount of the immunoconjugate to stimulate the immune system. In further embodiments, the invention provides a mutant IL-7 polypeptide for use in stimulating the immune system. In certain embodiments, the invention provides a mutant IL-7 polypeptide for use in a method of stimulating the immune system in an individual comprising administering to the individual an effective amount of the mutant IL-7 polypeptide to stimulate the immune system. An “individual” according to any of the above embodiments is a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides for the use of a mutant IL-7 polypeptide described herein or protein comprising such mutant IL-7 polypeptide, e.g., any of the immunoconjugates, in the manufacture or preparation of a medicament. In one embodiment, the medicament is for the treatment of a disease in an individual in need thereof. In one embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in an individual comprising administering to the individual an effective amount of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides a method for treating a disease in an individual. In one embodiment, the method comprises administering to an individual having such disease a therapeutically effective amount of a mutant IL-7 polypeptide described herein or protein comprising such mutant IL-7 polypeptide, e.g., any of the immunoconjugates. In one embodiment, a composition is administered to said individual, comprising the mutant IL-7 polypeptide or protein comprising such mutant IL-7 polypeptide, e.g., an immunoconjugate, in a pharmaceutically acceptable form. In certain embodiments, the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of a mutant IL-7 polypeptide or protein comprising a mutant IL-7 polypeptide, e.g., an immunoconjugate, to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using a mutant IL-7 polypeptide or protein comprising the mutant IL-7 polypeptide of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of kidney cancer, skin cancer, lung cancer, colorectal cancer, breast cancer, brain cancer, head and neck cancer, prostate cancer and bladder cancer. A skilled artisan readily recognizes that in many cases the mutant IL-7 polypeptide or protein comprising the mutant IL-7 polypeptide, such as an immunoconjugate, may not provide a cure but may only provide partial benefit. In some embodiments, a physiological change having some benefit is also considered therapeutically beneficial. Thus, in some embodiments, an amount of mutant IL-7 polypeptide or protein comprising the mutant IL-7 polypeptide, such as an immunoconjugate, that provides a physiological change is considered an “effective amount” or a “therapeutically effective amount”. The subject, patient, or individual in need of treatment is typically a mammal, more specifically a human.

In some embodiments, an effective amount of the mutant IL-7 polypeptide described herein or protein comprising such mutant IL-7 polypeptide, such as an immunoconjugate, is administered to a cell. In other embodiments, a therapeutically effective amount of the mutant IL-7 polypeptide described herein or protein comprising such mutant IL-7 polypeptide, such as an immunoconjugate, is administered to an individual for the treatment of disease.

For the prevention or treatment of disease, the appropriate dosage of the mutant IL-7 polypeptide described herein or protein comprising such mutant IL-7 polypeptide, such as an immunoconjugate, (when used alone or in combination with one or more other additional therapeutic agents) will depend on the type of disease to be treated, the route of administration, the body weight of the patient, the type of molecule (e.g. comprising an Fc domain or not), the severity and course of the disease, whether the mutant IL-7 polypeptide or protein comprising the mutant IL-7 polypeptide is administered for preventive or therapeutic purposes, previous or concurrent therapeutic interventions, the patient's clinical history and response to the mutant IL-7 polypeptide or protein comprising the mutant IL-7 polypeptide, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The mutant IL-7 polypeptide or protein comprising the mutant IL-7 polypeptide, e.g., the immunoconjugate, is suitably administered to the patient at one time or over a series of treatments. Depending on the type and severity of the disease, about 1 μg/kg to 15 mg/kg (e.g. 0.1 mg/kg-10 mg/kg) of mutant IL-7 polypeptide or protein comprising the mutant IL-7 polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the mutant IL-7 polypeptide or protein comprising the mutant IL-7 polypeptide would be in the range from about 0.005 mg/kg to about 10 mg/kg. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the mutant IL-7 polypeptide or protein comprising the mutant IL-7 polypeptide). An initial higher loading dose, followed by one or more lower doses may be administered. However, other dosage regimens may be useful. The progress of this therapy is easily monitored by conventional techniques and assays.

The mutant IL-7 polypeptide or protein comprising the mutant IL-7 polypeptide of the invention will generally be used in an amount effective to achieve the intended purpose. For use to treat or prevent a disease condition, the mutant IL-7 polypeptide or protein comprising the mutant IL-7 polypeptide of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the mutant IL-7 polypeptide or protein comprising the mutant IL-7 polypeptide which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the mutant IL-7 polypeptide or protein comprising the mutant IL-7 polypeptide may not be related to plasma concentration. One having skill in the art will be able to optimize therapeutically effective local dosages without undue experimentation.

A therapeutically effective dose of the mutant IL-7 polypeptide or protein comprising the mutant IL-7 polypeptide described herein will generally provide therapeutic benefit without causing substantial toxicity. Toxicity and therapeutic efficacy of an mutant IL-7 polypeptide or protein comprising the mutant IL-7 polypeptide can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Mutant IL-7 polypeptides or proteins comprising the mutant IL-7 polypeptide that exhibit large therapeutic indices are preferred. In one embodiment, the mutant IL-7 polypeptide or protein comprising the mutant IL-7 polypeptide according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with mutant IL-7 polypeptides or proteins comprising the mutant IL-7 polypeptide of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The maximum therapeutic dose of a protein comprising the mutant IL-7 polypeptide as described herein, e.g., an immunoconjugate, may be increased from those used for a protein comprising wild-type IL-7.

### Other Agents and Treatments

The mutant IL-7 polypeptides or proteins comprising the mutant IL-7 polypeptide according to the invention may be administered in combination with one or more other agents in therapy. For instance, a mutant IL-7 polypeptide or protein comprising the mutant IL-7 polypeptide of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of mutant IL-7 polypeptide or protein comprising the mutant IL-7 polypeptide used, the type of disorder or treatment, and other factors discussed above. The mutant IL-7 polypeptides or proteins comprising a mutant IL-7 polypeptide are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the mutant IL-7 polypeptide or protein comprising the mutant IL-7 polypeptide of the invention can occur prior to, simultaneously, and/or following, administration of the additional therapeutic agent and/or adjuvant. Mutant IL-7 polypeptides or proteins comprising a mutant IL-7 polypeptide of the invention may also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a mutant IL-7 polypeptide or protein comprising a mutant IL-7 polypeptide as described herein, such as an immunoconjugate of the invention. The label or package insert indicates that the composition is used for treating the condition of choice. Moreover, the article of manufacture may comprise (a) a first container with a composition contained therein, wherein the composition comprises a mutant IL-7 polypeptide or protein comprising a mutant IL-7 polypeptide as described herein, such as an immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Amino Acid Sequences

| | | SEQ ID |
|---|---|---|
| Description | Amino Acid Sequences | NOs |
| PD-1 FV000363 | DIVMTQSPDSLAVSLGERATINCKASESVDTSDNSFIHWYQQKPGQSPK | 1 |
| LC | LLIYRSSTLESGVPDRFSGSGSGTDFTLTISSLQAEDVAVYYCQQNYDVP | |
| | WTFGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKVYA | |
| | CEVTHQGLSSPVTKSFNRGEC | |
| PD-1 FV000363 | EVQLLESGGGLVQPGGSLRLSCAASGFSFSSYTMSWVRQAPGKGLEWV | 2 |
| HC hole | ATISGGGRDIYYPDSVKGRFTISRDNSKNTLYLQMNSLRAEDTAVYYCV | |
| | LLTGRVYFALDSWGQGTLVTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALHNH | |
| | YTQKSLSLSPG | |
| P1AH4515 | EVQLLESGGGLVQPGGSLRLSCAASGFSFSSYTMSWVRQAPGKGLEWV | 3 |
| HC knob | ATISGGGRDIYYPDSVKGRFTISRDNSKNTLYLQMNSLRAEDTAVYYCV | |
| | LLTGRVYFALDSWGQGTLVTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEALHN | |
| | HYTQKSLSLSPGGGGGSGGGGSGGGGSDCDIEGKDGKQYESVLMVSID | |
| | QLLDSMKEIGSNCLNNEFNFFKRHICDANKEGMFLFRAARKLRQFLKM | |
| | NSTGDFDLHLLKVSEGTTILLNCTGQVKGRKPAALGEAQPTKSLEENKS | |
| | LKEQKKLNDLCFLKRLLQEIKTCWNKILMGTKEH | |
| P1AG3724 | EVQLLESGGGLVQPGGSLRLSCAASGFSFSSYTMSWVRQAPGKGLEWV | 4 |
| HC knob | ATISGGGRDIYYPDSVKGRFTISRDNSKNTLYLQMNSLRAEDTAVYYCV | |
| | LLTGRVYFALDSWGQGTLVTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEALHN | |
| | HYTQKSLSLSPGGGGGSGGGGSGGGGDCDIEGKDGKQYESVLMVSIDQ | |
| | LLDSMKEIGSNCLNNEFNFFKRHICDANKEGMFLFRAARKLRQFLKMNS | |
| | TGDFDLHLLKVSEETTILLNCTGQVKGRKPAALGEAQPTKSLEENKSLK | |
| | EQKKLNDLCFLKRLLQEIKTCWNKILMGTKEH | |
| P1AG3725 | EVQLLESGGGLVQPGGSLRLSCAASGFSFSSYTMSWVRQAPGKGLEWV | 5 |
| HC knob | ATISGGGRDIYYPDSVKGRFTISRDNSKNTLYLQMNSLRAEDTAVYYCV | |
| | LLTGRVYFALDSWGQGTLVTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEALHN | |
| | HYTQKSLSLSPGGGGGSGGGGSGGGGDCDIEGKDGKQYESVLMVSIDQ | |
| | LLDSMKEIGSNCLNNEFNFFKRHICDANKEGMFLFRAARKLRQFLKMNS | |
| | TGDFDLHLLKVSEETTILLNCAGQVKGRKPAALGEAQPTKSLEENKALK | |
| | EQKKLNDLCFLKRLLQEIKTCWNKILMGTKEH | |
| P1AG3727 | EVQLLESGGGLVQPGGSLRLSCAASGFSFSSYTMSWVRQAPGKGLEWV | 6 |
| HC knob | ATISGGGRDIYYPDSVKGRFTISRDNSKNTLYLQMNSLRAEDTAVYYCV | |
| | LLTGRVYFALDSWGQGTLVTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEALHN | |
| | HYTQKSLSLSPGGGGGSGGGGSGGGGDCDIEGKDGKQYESVLMVSIDQ | |
| | LLDSMKEIGSNCLNNEFNFFKRHICDANKEGMFLFRAARKLRQFLKMNS | |
| | TGDFDLHLLEVSEETTILLNCAGQVKGRKPAALGEAQPTKSLEENKALK | |
| | EQKKLNDLCFLKRLLQEIKTCWNKILMGTKEH | |
| P1AH6010 | EVQLLESGGGLVQPGGSLRLSCAASGFSFSSYTMSWVRQAPGKGLEWVA | 7 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDKHL | |
| | LKVSEKTTILLNCTGQVKGRKPAALGEAQPTKSLEENKSLKEQKKLNDLC | |
| | FLKRLLQEIKTCWNKILMGTKEH | |
| P1AH6009 | EVQLLESGGGLVQPGGSLRLSCAASGFSFSSYTMSWVRQAPGKGLEWVA | 8 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDKHL | |
| | LKVSEGTTILLNCTGQVKGRKPAALGEAQPTKSLEENKSLKEQKKLNDLC | |
| | FLKRLLKEIKTCWNKILMGTKEH | |
| P1AH6008 | EVQLLESGGGLVQPGGSLRLSCAASGFSFSSYTMSWVRQAPGKGLEWVA | 9 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMKSIDQLLDSMKEI | |
| | GSNCLNNEFNFFKRHICDANKEGMFLFRAARKLRQFLKMNSTGDFDLHL | |
| | LKVSEGTTILLNCTGQVKGRKPAALGEAQPTKSLEENKSLKEQKKLNDLC | |
| | FLKRLLKEIKTCWNKILMGTKEH | |
| P1AH6007 | EVQLLESGGGLVQPGGSLRLSCAASGFSFSSYTMSWVRQAPGKGLEWVA | 10 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESKLMVSIDQLLDSMKEI | |
| | GSNCLNNEFNFFKRHICDANKEGMFLFRAARKLRQFLKMNSTGDFDLHL | |
| | LKVSEGTTILLNCTGQVKGRKPAALGEAQPTKSLEENKSLKEQKKLNDLC | |
| | FLKRLLKEIKTCWNKILMGTKEH | |
| P1AH6006 | EVQLLESGGGLVQPGGSLRLSCAASGFSFSSYTMSWVRQAPGKGLEWVA | 11 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LAVSEGTTILLNCTGQVKGRKPAALGEAQPTKSLEENKSLKEQKKLNDLC | |
| | FLKRLLKEIKTCWNKILMGTKEH | |
| P1AH6005 | EVQLLESGGGLVQPGGSLRLSCAASGFSFSSYTMSWVRQAPGKGLEWVA | 12 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGKFDLHL | |
| | LKVSEKTTILLNCTGQVKGRKPAALGEAQPTKSLEENKSLKEQKKLNDLC | |
| | FLKRLLQEIKTCWNKILMGTKEH | |
| P1AH6004 | EVQLLESGGGLVQPGGSLRLSCAASGFSFSSYTMSWVRQAPGKGLEWVA | 13 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMKSIDQLLDSMKEI | |
| | GSNCLNNEFNFFKRHICDANKEGMFLFRAARKLRQFLKMNSTGDFDLHL | |
| | LKVSEKTTILLNCTGQVKGRKPAALGEAQPTKSLEENKSLKEQKKLNDLC | |
| | FLKRLLQEIKTCWNKILMGTKEH | |
| P1AH6003HC | EVQLLESGGGLVQPGGSLRLSCAASGFSFSSYTMSWVRQAPGKGLEWVA | 14 |
| knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESALMVSIDQLLDSMKEI | |
| | GSNCLNNEFNFFKRHICDANKEGMFLFRAARKLRQFLKMNSTGDFDLHL | |
| | LKVSEKTTILLNCTGQVKGRKPAALGEAQPTKSLEENKSLKEQKKLNDLC | |
| | FLKRLLQEIKTCWNKILMGTKEH | |
| P1AH6002 | EVQLLESGGGLVQPGGSLRLSCAASGFSFSSYTMSWVRQAPGKGLEWVA | 15 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESALMVSIDQLLDSMKEI | |
| | GSNCLNNEFNFFKRHICDANKEGMFLFRAARKLRQFLKMNSTGDFDLHL | |
| | LAVSEGTTILLNCTGQVKGRKPAALGEAQPTKSLEENKSLKEQKKLNDLC | |
| | FLKRLLQEIKTCWNKILMGTKEH | |
| P1AH6017 | EVQLLESGGGLVQPGGSLRLSCAASGFSFSSYTMSWVRQAPGKGLEWVA | 16 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEK | |
| P1AH6016 | EVQLLESGGGLVQPGGSLRLSCAASGFSFSSYTMSWVRQAPGKGLEWVA | 17 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKKH | |
| P1AH6047 | EVQLLESGGGLVQPGGSLRLSCAASGFSFSSYTMSWVRQAPGKGLEWVA | 18 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKKNDLC | |
| | FLKRLLQEIKTCWNKILMGTKEH | |
| P1AH6046 | EVQLLESGGGLVQPGGSLRLSCAASGFSFSSYTMSWVRQAPGKGLEWVA | 19 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAAQGEAQPTKSQEENKSLKEQKKLNDLC | |
| | FLKRLLQEIKTCWNKILMGTKEH | |
| P1AH6045 | EVQLLESGGGLVQPGGSLRLSCAASGFSFSSYTMSWVRQAPGKGLEWVA | 20 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSKEENKSLKEQKKLNDLC | |
| | FLKRLLQEIKTCWNKILMGTKEH | |
| P1AH6044 | EVQLLESGGGLVQPGGSLRLSCAASGFSFSSYTMSWVRQAPGKGLEWVA | 21 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAAKGEAQPTKSLEENKSLKEQKKLNDLC | |
| | FLKRLLQEIKTCWNKILMGTKEH | |
| P1AH6043 | EVQLLESGGGLVQPGGSLRLSCAASGFSFSSYTMSWVRQAPGKGLEWVA | 22 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | KEVSEETTILLNCTGQVKGRKPAALGEAQPTKSLEENKSLKEQKKLNDLC | |
| | FLKRLLQEIKTCWNKILMGTKEH | |
| P1AH6042 | EVQLLESGGGLVQPGGSLRLSCAASGFSFSSYTMSWVRQAPGKGLEWVA | 23 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFKKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6041 | EVQLLESGGGLVQPGGSLRLSCAASGFSFSSYTMSWVRQAPGKGLEWVA | 24 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEK | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6025 | EVQLLESGGGLVQPGGSLRLSCAASGFSFSSYTMSWVRQAPGKGLEWVA | 25 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDKHL | |
| | LKVSEETTILLNCTGQVKGRKPAALGEAQPTKSLEENKSLKEQKKLNDLC | |
| | FLKRLLQEIKTCWNKILMGTKEH | |
| P1AH6021 | EVQLLESGGGLVQPGGSLRLSCAASGFSFSSYTMSWVRQAPGKGLEWVA | 26 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYKSVLMVSIDQLLDSMKEI | |
| | GSNCLNNEFNFFKRHICDANKEGMFLFRAARKLRQFLKMNSTGDFDLHL | |
| | LKVSEETTILLNCTGQVKGRKPAALGEAQPTKSLEENKSLKEQKKLNDLC | |
| | FLKRLLQEIKTCWNKILMGTKEH | |
| P1AH6020 | EVQLLESGGGLVQPGGSLRLSCAASGFSFSSYTMSWVRQAPGKGLEWVA | 27 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LQVSEQTTILLNCTGQVKGRKPAALGEAQPTKSLEENKSLKEQKKLNDLC | |
| | FLKRLLQEIKTCWNKILMGTKEH | |
| P1AH6019 | EVQLLESGGGLVQPGGSLRLSCAASGFSFSSYTMSWVRQAPGKGLEWVA | 28 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEQTTILLNCTGQVKGRKPAALGEAQPTKSLEENKSLKEQKKLNDLC | |
| | FLKRLLQEIKTCWNKILMGTKEH | |
| P1AH6018 | EVQLLESGGGLVQPGGSLRLSCAASGFSFSSYTMSWVRQAPGKGLEWVA | 29 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LQVSEETTILLNCTGQVKGRKPAALGEAQPTKSLEENKSLKEQKKLNDLC | |
| | FLKRLLQEIKTCWNKILMGTKEH | |
| P1AH6039 | EVQLLESGGGLVQPGGSLRLSCAASGFSFSSYTMSWVRQAPGKGLEWVA | 30 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKNGTQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSNETNKSLKEQKKLNDLC | |
| | FLKRLLQEIKTCWNKILMGTKEH | |
| P1AH6038 | EVQLLESGGGLVQPGGSLRLSCAASGFSFSSYTMSWVRQAPGKGLEWVA | 31 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKNGTQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENGTLKEQKKLNDLC | |
| | FLKRLLQEIKTCWNKILMGTKEH | |
| P1AH6037 | EVQLLESGGGLVQPGGSLRLSCAASGFSFSSYTMSWVRQAPGKGLEWVA | 32 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNQNFTFFKRHICDANKEGMFLFRAARKLRQFLKMNSTGDFDLHL | |
| | LEVSEETTILLNCTGQVKGRKPAALGEAQPNKTLEENKSLKEQKKLNDLC | |
| | FLKRLLQEIKTCWNKILMGTKEH | |
| P1AH6036 | EVQLLESGGGLVQPGGSLRLSCAASGFSFSSYTMSWVRQAPGKGLEWVA | 33 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNNFTFFKRHICDANKEGMFLFRAARKLRQFLKMNSTGDFDLHL | |
| | LEVSEETTILLNCTGQVKGRKPAALGNATSTKSLEENKSLKEQKKLNDLC | |
| | FLKRLLQEIKTCWNKILMGTKEH | |
| P1AH6035 | EVQLLESGGGLVQPGGSLRLSCAASGFSFSSYTMSWVRQAPGKGLEWVA | 34 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKNGTQYESVLMVSIDQLLDSMKEI | |
| | GSNCLNNEFNFFKRHICDANKNGTFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6034 | EVQLLESGGGLVQPGGSLRLSCAASGFSFSSYTMSWVRQAPGKGLEWVA | 35 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCNITGKDGKQYESVLMVSIDQLLDSMKEI | |
| | GSNCLNNEFNFFKRHICDANKNGTFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6033 | EVQLLESGGGLVQPGGSLRLSCAASGFSFSSYTMSWVRQAPGKGLEWVA | 36 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTLNGTSTST | |
| P1AH6032 | EVQLLESGGGLVQPGGSLRLSCAASGFSFSSYTMSWVRQAPGKGLEWVA | 37 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSNETNKSLKEQKKLNDLC | |
| | FLKRLLQEIKTCWNKILMGTKEH | |
| P1AH6031 | EVQLLESGGGLVQPGGSLRLSCAASGFSFSSYTMSWVRQAPGKGLEWVA | 38 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPNKTLEENKSLKEQKKLNDLC | |
| | FLKRLLQEIKTCWNKILMGTKEH | |
| P1AH6030 | EVQLLESGGGLVQPGGSLRLSCAASGFSFSSYTMSWVRQAPGKGLEWVA | 39 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGNATSTKSLEENKSLKEQKKLNDLC | |
| | FLKRLLQEIKTCWNKILMGTKEH | |
| P1AH6029 | EVQLLESGGGLVQPGGSLRLSCAASGFSFSSYTMSWVRQAPGKGLEWVA | 40 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKNGTFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6028 | EVQLLESGGGLVQPGGSLRLSCAASGFSFSSYTMSWVRQAPGKGLEWVA | 41 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICNATKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6027 | EVQLLESGGGLVQPGGSLRLSCAASGFSFSSYTMSWVRQAPGKGLEWVA | 42 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNQNFTFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6026 | EVQLLESGGGLVQPGGSLRLSCAASGFSFSSYTMSWVRQAPGKGLEWVA | 43 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNNFTFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6024 | EVQLLESGGGLVQPGGSLRLSCAASGFSFSSYTMSWVRQAPGKGLEWVA | 44 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCNITGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH6023 | EVQLLESGGGLVQPGGSLRLSCAASGFSFSSYTMSWVRQAPGKGLEWVA | 45 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKNGTQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH4506 | EVQLLESGGGLVQPGGSLRLSCAASGFSFSSYTMSWVRQAPGKGLEWVA | 46 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGGGSGGGGSGGGGSDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AH4508 | EVQLLESGGGLVQPGGSLRLSCAASGFSFSSYTMSWVRQAPGKGLEWVA | 47 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGGGSGGGGSGGGGSDCDIEGKDGKQYESVLMVSIDQLLDSMKEI | |
| | GSNCLNNEFNFFKRHICDANKEGMFLFRAARKLRQFLKMNSTGDFDLHL | |
| | LKVSEGTTILLNCTGQVKGRKPAALGEAQPTKSLEENKSLKEQKKLNDLC | |
| | FLKRLLQEIKTCWNKILMGTKEH | |
| P1AH4502 | EVQLLESGGGLVQPGGSLRLSCAASGFSFSSYTMSWVRQAPGKGLEWVA | 48 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGGGSGGGGSGGGGDCDIEGKDGKQYESVLMVSIDQLLDSMKEIG | |
| | SNCLNNEFNFFKRHICDANKEGMFLFRAARKLRQFLKMNSTGDFDLHLL | |
| | KVSEGTTILLNCTGQVKGRKPAALGEAQPTKSLEENKSLKEQKKLNDLCF | |
| | LKRLLQEIKTCWNKILMGTKEH | |
| P1AH4515 | EVQLLESGGGLVQPGGSLRLSCAASGFSFSSYTMSWVRQAPGKGLEWVA | 49 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LKVSEGTTILLNCTGQVKGRKPAALGEAQPTKSLEENKSLKEQKKLNDLC | |
| | FLKRLLQEIKTCWNKILMGTKEH | |
| P1AH4513 | EVQLLESGGGLVQPGGSLRLSCAASGFSFSSYTMSWVRQAPGKGLEWVA | 50 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AG3048 | EVQLLESGGGLVQPGGSLRLSCAASGFSFSSYTMSWVRQAPGKGLEWVA | 51 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGGGSGGGGSGGGGDCDIEGKDGKQYESVLMVSIDQLLDSMKEIG | |
| | SNCLNNEFNFFKRHICDANKEGMFLFRAARKLRQFLKMNSTGDFDLHLL | |
| | EVSEETTILLNCTGQVKGRKPAALGEAQPTKSLEENKSLKEQKKLNDLCF | |
| | LKRLLQEIKTCWNKILMGTKEH | |
| P1AG0950 | EVQLLESGGGLVQPGGSLRLSCAASGFSFSSYTMSWVRQAPGKGLEWVA | 52 |
| HC knob | TISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGGGSGGGGSGGGGSDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| PD-1 FV020519 | DIVMTQSPDSLAVSLGERATINCKASESVDTSDNSFIHWYQQKPGQSPKL | 53 |
| LC | LIYRASTLESGVPDRFSGSGSGTDFTLTISSLQAEDVAVYYCQQNYDVPW | |
| | TFGQGTKVEIKRTVAAPSVFIFPPSDEQLKSGTASVVCLLNNFYPREAKVQ | |
| | WKVDNALQSGNSQESVTEQDSKDSTYSLSSTLTLSKADYEKHKVYACEV | |
| | THQGLSSPVTKSFNRGEC | |
| PD-1 FV020519 | EVQLLESGGGLVQPGGSLRLSCAASGFSFSSYTMSWVRQAPGKGLEWVS | 54 |
| HC hole | AISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVCTLPPSRDELTKNQVSLSCAVKGFYPSDIAVEWESNGQPENNYKTT | |
| | PPVLDSDGSFFLVSKLTVDKSRWQQGNVFSCSVMHEALHNHYTQKSLSL | |
| | SPG | |
| P1AJ0773 | EVQLLESGGGLVQPGGSLRLSCAASGFSFSSYTMSWVRQAPGKGLEWVS | 55 |
| HC knob | AISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGDFDLHL | |
| | LEVSEETTILLNCTGQVKGRKPAALGEAQPTKSLEENKSLKEQKKLNDLC | |
| | FLKRLLQEIKTCWNKILMGTKEH | |
| P1AI5585 | EVQLLESGGGLVQPGGSLRLSCAASGFSFSSYTMSWVRQAPGKGLEWVS | 56 |
| HC knob | AISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYESVLMVSIDQLLDSMKEI | |
| | GSNCLNNEFNFFKRHICDANKEGMFLFRAARKLRQFLKMNSTGKFDLHL | |
| | LKVSEKTTILLNCTGQVKGRKPAALGEAQPTKSLEENKSLKEQKKLNDLC | |
| | FLKRLLQEIKTCWNKILMGTKEH | |
| P1AI5587 | EVQLLESGGGLVQPGGSLRLSCAASGFSFSSYTMSWVRQAPGKGLEWVS | 57 |
| HC knob | AISGGGRDIYYPDSVKGRFTISRDNSKNTLYLQMNSLRAEDTAVYYCVLL | |
| | TGRVYFALDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKP | |
| | KDTLMISRTPEVTCVVVDVSHEDPEVKFNWYVDGVEVHNAKTKPREEQ | |
| | YNSTYRVVSVLTVLHQDWLNGKEYKCKVSNKALGAPIEKTISKAKGQPR | |
| | EPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLS | |
| | LSPGGGSGGGGSGGGGSGGDCDIEGKDGKQYKSVLMVSIDQLLDSMKEI | |
| | GSNCLNNEFNFFKRHICDANKEGMFLFRAARKLRQFLKMNSTGDFDLHL | |
| | LKVSEETTILLNCTGQVKGRKPAALGEAQPTKSLEENKSLKEQKKLNDLC | |
| | FLKRLLQEIKTCWNKILMGTKEH | |
| P1AF7303 LC1 | EIVLTQSPGTLSLSPGERATLSCRASQSVTSSYLAWYQQKPGQAPRLLINV | 58 |
| | GSRRATGIPDRFSGSGSGTDFTLTISRLEPEDFAVYYCQQGIMLPPTFGQGT | |
| | KVEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLS | |
| | SPVTKSFNRGEC | |
| P1AF7303 HC | EVQLLESGGGLVQPGGSLRLSCAASGFTFSSYAMSWVRQAPGKGLEWVS | 59 |
| hole | AIIGSGASTYYADSVKGRFTISRDNSKNTLYLQMNSLRAEDTAVYYCAKG | |
| | WFGGFNYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQTYI | |
| | CNVNHKPSNTKVDKKVEPKSCDKTHTCPPCPAPEAAGGPSVFLFPPKPKD | |
| | TLMISRTPEVTCVVVDVSHEDPEVKFNWYVDGVEVHNAKTKPREEQYNS | |
| | TYRVVSVLTVLHQDWLNGKEYKCKVSNKALGAPIEKTISKAKGQPREPQ | |
| | VCTLPPSRDELTKNQVSLSCAVKGFYPSDIAVEWESNGQPENNYKTTPPV | |
| | LDSDGSFFLVSKLTVDKSRWQQGNVFSCSVMHEALHNHYTQKSLSLSPG | |
| | K | |
| P1AF7303 HC | EVQLLESGGGLVQPGGSLRLSCAASGFTFSSYAMSWVRQAPGKGLEWVS | 60 |
| knob | AIIGSGASTYYADSVKGRFTISRDNSKNTLYLQMNSLRAEDTAVYYCAKG | |
| | WFGGFNYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQTYI | |
| | CNVNHKPSNTKVDKKVEPKSCDKTHTCPPCPAPEAAGGPSVFLFPPKPKD | |
| | TLMISRTPEVTCVVVDVSHEDPEVKFNWYVDGVEVHNAKTKPREEQYNS | |
| | TYRVVSVLTVLHQDWLNGKEYKCKVSNKALGAPIEKTISKAKGQPREPQ | |
| | VYTLPPCRDELTKNQVSLWCLVKGFYPSDIAVEWESNGQPENNYKTTPP | |
| | VLDSDGSFFLYSKLTVDKSRWQQGNVFSCSVMHEALHNHYTQKSLSLSP | |
| | GGGGGSGGGGSGGGGSDCDIEGKDGKQYESVLMVSIDQLLDSMKEIGSN | |
| | CLNNEFNFFKRHICDANKEGMFLFRAARKLRQFLKMNSTGDFDLHLLKV | |
| | SEGTTILLNCTGQVKGRKPAALGEAQPTKSLEENKSLKEQKKLNDLCFLK | |
| | RLLQEIKTCWNKILMGTKEH | |
| PD1 FV012014 LC | DVVMTQSPLSLPVTLGQPASISCRSSQSLVHANTNTYLEWYQQRPGQSPR | 61 |
| | LLIYKVSNRFSGVPDRFSGSGSGTDFTLKISRVEAEDVGVYYCFQGTHVP | |
| | NTFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYACE | |
| | VTHQGLSSPVTKSFNRGEC | |
| PD1 FV012014 HC | QIQLVQSGSELKKPGASVKVSCKASGYTFTHYAMNWVRQAPGQGLEWM | 62 |
| knob plus IL7 | GWINTNTGEPTYAQGFTGRFVFSLDTSVSTAYLQISSLKAEDTAVYYCAR | |
| | EREPGMDNWGQGTLVTVSSASTKGPSVFPLAPSSKSTSGGTAALGCLVK | |
| | DYFPEPVTVSWNSGALTSGVHTFPAVLQSSGLYSLSSVVTVPSSSLGTQTY | |
| | ICNVNHKPSNTKVDKKVEPKSCDKTHTCPPCPAPEAAGGPSVFLFPPKPK | |
| | DTLMISRTPEVTCVVVDVSHEDPEVKFNWYVDGVEVHNAKTKPREEQY | |
| | NSTYRVVSVLTVLHQDWLNGKEYKCKVSNKALGAPIEKTISKAKGQPRE | |
| | PQVYTLPPCRDELTKNQVSLWCLVKGFYPSDIAVEWESNGQPENNYKTT | |
| | PPVLDSDGSFFLYSKLTVDKSRWQQGNVFSCSVMHEALHNHYTQKSLSL | |
| | SPGGGSGGGGSGGGGSGDCDIEGKDGKQYESVLMVSIDQLLDSMKEIGS | |
| | NCLNNEFNFFKRHICDANKEGMFLFRAARKLRQFLKMNSTGDFDLHLLK | |
| | VSEGTTILLNCTGQVKGRKPAALGEAQPTKSLEENKSLKEQKKLNDLCFL | |
| | KRLLQEIKTCHNKILMGTKEH | |
| P1AK9505 HC | DKTHTCPPCPAPEAAGGPSVFLFPPKPKDTLMISRTPEVTCVVVDVSHEDP | 63 |
| hole | EVKFNWYVDGVEVHNAKTKPREEQYNSTYRVVSVLTVLHQDWLNGKE | |
| | YKCKVSNKALGAPIEKTISKAKGQPREPQVCTLPPSRDELTKNQVSLSCA | |
| | VKGFYPSDIAVEWESNGQPENNYKTTPPVLDSDGSFFLVSKLTVDKSRW | |
| | QQGNVFSCSVMHEALHNRFTQKSLSLSPG | |
| human IL7 wild- | DCDIEGKDGKQYESVLMVSIDQLLDSMKEIGSNCLNNEFNFFKRHICDAN | 64 |
| type | KEGMFLFRAARKLRQFLKMNSTGDFDLHLLKVSEGTTILLNCTGQVKGR | |
| | KPAALGEAQPTKSLEENKSLKEQKKLNDLCFLKRLLQEIKTCWNKILMGT | |
| | KEH | |
| IL7v2.2 (D74K, | DCDIEGKDGKQYESVLMVSIDQLLDSMKEIGSNCLNNEFNFFKRHICDAN | 65 |
| G85K) | KEGMFLFRAARKLRQFLKMNSTGKFDLHLLKVSEKTTILLNCTGQVKGR | |
| | KPAALGEAQPTKSLEENKSLKEQKKLNDLCFLKRLLQEIKTCWNKILMGT | |
| | KEH | |
| IL7v2.6(E13K, | DCDIEGKDGKQYKSVLMVSIDQLLDSMKEIGSNCLNNEFNFFKRHICDAN | 66 |
| G85E) | KEGMFLFRAARKLRQFLKMNSTGDFDLHLLKVSEETTILLNCTGQVKGR | |
| | KPAALGEAQPTKSLEENKSLKEQKKLNDLCFLKRLLQEIKTCWNKILMGT | |
| | KEH | |
| CDR-H1 of de- | SYTMS | 67 |
| risked PD-1 binder | | |
| acc. to Kabbat | | |
| CDR-H2 of de- | AISGGGRDIYYPDSVKG | 68 |
| risked PD-1 binder | | |
| acc. to Kabbat | | |
| CDR-H3 of de- | LTGRVYFALDS | 69 |
| risked PD-1 binder | | |
| acc. to Kabbat | | |
| CDR-L1 of de- | KASESVDTSDNSFIH | 70 |
| risked PD-1 binder | | |
| acc. to Kabbat | | |
| CDR-L2 of de- | RASTLES | 71 |
| risked PD-1 binder | | |
| acc. to Kabbat | | |
| CDR-L3 of de- | QQNYDVPWT | 72 |
| risked PD-1 binder | | |
| acc. to Kabbat | | |
| VH of de-risked | EVQLLESGGGLVQPGGSLRLSCAASGFSFSSYTMSWVRQAPGKGLEWVS | 73 |
| PD1 binder | AISGGGRDIYYPDSVKGRFTISRDNSKNTLYLQMNSLRAEDTAVYYCVLL | |
| | TGRVYFALDSWGQGTLVTVSS | |
| VLof de-risked | DIVMTQSPDSLAVSLGERATINCKASESVDTSDNSFIHWYQQKPGQSPKL | 74 |
| PD1 binder | LIYRASTLESGVPDRFSGSGSGTDFTLTISSLQAEDVAVYYCQQNYDVPW | |
| | TFGQGTKVEIK | |
| Linker | GGSGGGGSGGGGSGG | 75 |
| P1AL9335 LC | DIVMTQSPDSLAVSLGERATINCKASESVDTSDNSFIHWYQQKPGQSPKL | 76 |
| | LIYRASTLESGVPDRFSGSGSGTDFTLTISSLQAEDVAVYYCQQNYDVPW | |
| | TFGQGTKVEIKRADAAPTVSIFPPSSEQLTSGGASVVCFLNNFYPKDINVK | |
| | WKIDGSERQNGVLNSWTDQDSKDSTYSMSSTLTLTKDEYERHNSYTCEA | |
| | THKTSTSPIVKSFNRGEC | |
| P1AL9335 HC 1 | EVQLLESGGGLVQPGGSLRLSCAASGFSFSSYTMSWVRQAPGKGLEWVS | 77 |
| | AISGGGRDIYYPDSVKGRFTISRDNSKNTLYLQMNSLRAEDTAVYYCVLL | |
| | TGRVYFALDSWGQGTLVTVSSAKTTPPSVYPLAPGSAAQTNSMVTLGCL | |
| | VKGYFPEPVTVTWNSGSLSSGVHTFPAVLQSDLYTLSSSVTVPSSTWPSQ | |
| | TVTCNVAHPASSTKVDKKIEPKSCGCKPCICTVPEVSSVFIFPPKPKDVLTI | |
| | TLTPKVTCVVVAISKDDPEVQFSWFVDDVEVHTAQTKPREEQINSTFRSV | |
| | SELPIMHQDWLNGKEFKCRVNSAAFGAPIEKTISKTKGRPKAPQVYTIPPP | |
| | KKQMAKDKVSLTCMITNFFPEDITVEWQWNGQPAENYKNTQPIMKTDG | |
| | SYFVYSKLNVQKSNWEAGNTFTCSVLHEGLHNHHTEKSLSHSPG | |
| P1AL9335 | EVQLLESGGGLVQPGGSLRLSCAASGFSFSSYTMSWVRQAPGKGLEWVS | 78 |
| HC 2 | AISGGGRDIYYPDSVKGRFTISRDNSKNTLYLQMNSLRAEDTAVYYCVLL | |
| | TGRVYFALDSWGQGTLVTVSSAKTTPPSVYPLAPGSAAQTNSMVTLGCL | |
| | VKGYFPEPVTVTWNSGSLSSGVHTFPAVLQSDLYTLSSSVTVPSSTWPSQ | |
| | TVTCNVAHPASSTKVDKKIEPKSCGCKPCICTVPEVSSVFIFPPKPKDVLTI | |
| | TLTPKVTCVVVAISKDDPEVQFSWFVDDVEVHTAQTKPREEQINSTFRSV | |
| | SELPIMHQDWLNGKEFKCRVNSAAFGAPIEKTISKTKGRPKAPQVYTIPPP | |
| | KEQMAKDKVSLTCMITNFFPEDITVEWQWNGQPAENYDNTQPIMDTDGS | |
| | YFVYSDLNVQKSNWEAGNTFTCSVLHEGLHNHHTEKSLSHSPGGGSGGG | |
| | GSGGGGSGGDCDIEGKDGKQYKSVLMVSIDQLLDSMKEIGSNCLNNEFN | |
| | FFKRHICDANKEGMFLFRAARKLRQFLKMNSTGDFDLHLLKVSEETTILL | |
| | NCTGQVKGRKPAALGEAQPTKSLEENKSLKEQKKLNDLCFLKRLLQEIKT | |
| | CWNKILMGTKEH | |
| Murinized | EIVLTQSPATLSLSPGERATLSCRASKGVSTSGYSYLHWYQQKPGQAPRL | 79 |
| Pembrolizumab LC | LIYLASYLESGVPARFSGSGSGTDFTLTISSLEPEDFAVYYCQHSRDLPLTF | |
| | GGGTKVEIKRADAAPTVSIFPPSSEQLTSGGASVVCFLNNFYPKDINVKW | |
| | KIDGSERQNGVLNSWTDQDSKDSTYSMSSTLTLTKDEYERHNSYTCEAT | |
| | HKTSTSPIVKSFNRNEC | |
| Murinized | QVQLVQSGVEVKKPGASVKVSCKASGYTFTNYYMYWVRQAPGQGLEW | 80 |
| Pembrolizumab HC | MGGINPSNGGTNFNEKFKNRVTLTTDSSTTTAYMELKSLQFDDTAVYYC | |
| | ARRDYRFDMGFDYWGQGTTVTVSSAKTTPPSVYPLAPGSAAQTNSMVT | |
| | LGCLVKGYFPEPVTVTWNSGSLSSGVHTFPAVLQSDLYTLSSSVTVPSST | |
| | WPSQTVTCNVAHPASSTKVDKKIVPRDCGCKPCICTVPEVSSVFIFPPKPK | |
| | DVLTITLTPKVTCVVVAISKDDPEVQFSWFVDDVEVHTAQTKPREEQINS | |
| | TFRSVSELPIMHQDWLNGKEFKCRVNSAAFGAPIEKTISKTKGRPKAPQV | |
| | YTIPPPKEQMAKDKVSLTCMITNFFPEDITVEWQWNGQPAENYKNTQPIM | |
| | DTDGSYFVYSKLNVQKSNWEAGNTFTCSVLHEGLHNHHTEKSLSHSPG | |

### Further Embodiments

Embodiment 1. A mutant interleukin-7 (IL-7) polypeptide comprising the amino acid substitutions E13K and G85E, wherein the numbering is relative to the human IL-7 sequence of SEQ ID NO: 64.

Embodiment 2. The mutant IL-7 polypeptide according to Embodiment 1, wherein the binding affinity to IL-7Rα is reduced as compared to an interleukin-7 polypeptide comprising the sequence of SEQ ID NO: 64.

Embodiment 3. The mutant IL-7 polypeptide according to Embodiment 1, wherein the binding affinity to IL-7Rα is reduced as compared to a reference IL-7 polypeptide which is identical to the mutant IL-7 except that it has E at position 13 and G at position 85, wherein the numbering is relative to the human IL-7 sequence of SEQ ID NO: 64.

Embodiment 4. The mutant IL-7 polypeptide according to Embodiment 2 or 3, wherein the binding affinity to IL-7Rα is reduced at least a 5-fold, at least a 10-fold or at least a 25-fold.

Embodiment 5. A mutant interleukin-7 (IL-7) polypeptide, comprising at least two amino acid substitutions at the position of E13 and G85 of human IL-7 according to SEQ ID NO: 64, wherein the amino acid substitution reduces the binding affinity of the mutant interleukin-7 polypeptide to IL-7Rα compared to an interleukin-7 polypeptide comprising the sequence of SEQ ID NO: 64, wherein the at least two amino acid substitutions are E13K and G85E.

Embodiment 6. The mutant IL-7 polypeptide according to any one of Embodiments 1 to 5, wherein the mutant IL-7 polypeptide comprises an amino acid sequence that is at least about 70%, at least about 75%, at least about 80%, at least about 85%, at least about 90%, at least about 91%, at least about 92%, at least about 93%, at least about 94%, at least about 95%, at least about 96% or 100% identical to SEQ ID NO: 66.

Embodiment 7. The mutant IL-7 polypeptide according to any one of Embodiments 1 to 5, wherein the mutant IL-7 polypeptide comprises the amino acid sequence of SEQ ID NO: 66.

Embodiment 8. A mutant interleukin-7 (IL-7) polypeptide comprising the amino acid substitutions D74K and G85K, wherein the numbering is relative to the human IL-7 sequence of SEQ ID NO: 64.

Embodiment 9. The mutant IL-7 polypeptide according to Embodiment 8, wherein the binding affinity to IL-7Rα is reduced as compared to an interleukin-7 polypeptide comprising the sequence of SEQ ID NO: 64.

Embodiment 10. The mutant IL-7 polypeptide according to Embodiment 8, wherein the binding affinity to IL-7Rα is reduced as compared to a reference IL-7 polypeptide which is identical to the mutant IL-7 except that it has D at position 74 and G at position 85, wherein the numbering is relative to the human IL-7 sequence of SEQ ID NO: 64.

Embodiment 11. The mutant IL-7 polypeptide according to Embodiment 9 or 10, wherein the binding affinity to IL-7Rα is reduced at least a 5-fold, at least a 10-fold or at least a 25-fold.

Embodiment 12. A mutant interleukin-7 (IL-7) polypeptide, comprising at least two amino acid substitutions at the position of D74 and G85 of human IL-7 according to SEQ ID NO: 64, wherein the amino acid substitution reduces the binding affinity of the mutant interleukin-7 polypeptide to IL-7Rα compared to an interleukin-7 polypeptide comprising the sequence of SEQ ID NO: 64, wherein the at least two amino acid substitutions are D74K and G85K.

Embodiment 13. The mutant IL-7 polypeptide according to any one of Embodiments 8 to 12, wherein the mutant IL-7 polypeptide comprises an amino acid sequence that is at least about 70%, at least about 75%, at least about 80%, at least about 85%, at least about 90%, at least about 91%, at least about 92%, at least about 93%, at least about 94%, at least about 95%, at least about 96% or 100% identical to SEQ ID NO: 65.

Embodiment 14. The mutant IL-7 polypeptide according to any one of Embodiments 8 to 12, wherein the mutant IL-7 polypeptide comprises the amino acid sequence of SEQ ID NO: 65.

Embodiment 15. A protein comprising the mutant IL-7 polypeptide of any one of Embodiments 1 to 14.

Embodiment 16. An immunoconjugate comprising (i) a mutant IL-7 polypeptide of any one of Embodiments 1 to 14 and (ii) an antibody.

Embodiment 17. The immunoconjugate according to Embodiment 16, wherein the antibody binds to PD-1.

Embodiment 18. An immunoconjugate according to Embodiments 16 or 17, wherein the antibody comprises (a) a heavy chain variable region (VH) comprising a CDR-H1 comprising the amino acid sequence of SEQ ID NO:67, a CDR-H2 comprising the amino acid sequence of SEQ ID NO: 68, a CDR-H3 comprising the amino acid sequence of SEQ ID NO:69, and (b) a light chain variable region (VL) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

Embodiment 19. An immunoconjugate according to any one of Embodiments 16 to 18, wherein the antibody comprises (a) a heavy chain variable region (VH) comprising an amino acid sequence that is at least about 95%, at least about 96%, at least about 97%, at least about 98%, at least about 99% or 100% identical to the amino acid sequence of SEQ ID NO:73, and (b) a light chain variable region (VL) comprising an amino acid sequence that is at least about 95%, at least about 96%, at least about 97%, at least about 98%, at least about 99% or 100% identical to the amino acid sequence of SEQ ID NO:74.

Embodiment 20. An immunoconjugate according to any one of Embodiments 16 to 18, wherein the antibody comprises (a) a heavy chain variable region (VH) comprising the amino acid sequence of SEQ ID NO:73, and (b) a light chain variable region (VL) comprising the amino acid sequence of SEQ ID NO: 74.

Embodiment 21. An immunoconjugate according to Embodiments 16 or 17, wherein the antibody comprises (a) two heavy chain variable regions (VH), each comprising a CDR-H1 comprising the amino acid sequence of SEQ ID NO:67, a CDR-H2 comprising the amino acid sequence of SEQ ID NO: 68, a CDR-H3 comprising the amino acid sequence of SEQ ID NO:69, and (b) two light chain variable regions (VL), each comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

Embodiment 22. An immunoconjugate according to any one of Embodiments 16, 17 and 21, wherein the antibody comprises (a) two heavy chain variable regions (VH), each comprising an amino acid sequence that is at least about 95%, at least about 96%, at least about 97%, at least about 98%, at least about 99% or 100% identical to the amino acid sequence of SEQ ID NO:73, and (b) two light chain variable regions (VL), each comprising an amino acid sequence that is at least about 95%, at least about 96%, at least about 97%, at least about 98%, at least about 99% or 100% identical to the amino acid sequence of SEQ ID NO:74.

Embodiment 23. An immunoconjugate according to any one of Embodiments 16, 17 and 21, wherein the antibody comprises (a) two heavy chain variable regions (VH), each comprising an amino acid sequence that is identical to the amino acid sequence of SEQ ID NO:73, and two light chain variable regions (VL), each comprising an amino acid sequence that is identical to the amino acid sequence of SEQ ID NO:74.

Embodiment 24. The immunoconjugate of any one of Embodiments 16 to 23, wherein the immunoconjugate comprises not more than one IL-7 polypeptide and/or not more than one antibody.

Embodiment 25. The immunoconjugate of any one of Embodiments 16 to 24, wherein the antibody comprises an Fc domain composed of a first and a second subunit.

Embodiment 26. The immunoconjugate of Embodiment 25, wherein the Fc domain is an IgG class, particularly an IgG1 subclass, Fc domain.

Embodiment 27. The immunoconjugate of Embodiment 25 or 26, wherein the Fc domain is a human Fc domain.

Embodiment 28. The immunoconjugate of any one of Embodiments 16 to 27, wherein the antibody is an IgG class, particularly an IgG1 subclass immunoglobulin.

Embodiment 29. The immunoconjugate of any one of Embodiments 25 to 28, wherein the Fc domain comprises a modification promoting the association of the first and the second subunit of the Fc domain.

Embodiment 30. The immunoconjugate of any one of Embodiments 25 to 29, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Embodiment 31. The immunoconjugate of any one of Embodiments 25 to 30, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index).

Embodiment 32. The immunoconjugate of Embodiment 31, wherein in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

Embodiment 33. The immunoconjugate of any one of Embodiments 25 to 32, wherein the mutant IL-7 polypeptide is fused at its amino-terminal amino acid to the carboxy-terminal amino acid of one of the subunits of the Fc domain, particularly the first subunit of the Fc domain, optionally through a linker peptide.

Embodiment 34. The immunoconjugate of Embodiment 33, wherein the linker peptide has the amino acid sequence of SEQ ID NO: 75.

Embodiment 35. The immunoconjugate of any one of Embodiments 25 to 34, wherein the Fc domain comprises one or more amino acid substitution that reduces binding to an Fc receptor, particularly an Fcγ receptor, and/or effector function, particularly antibody-dependent cell-mediated cytotoxicity (ADCC).

Embodiment 36. The immunoconjugate of Embodiment 35, wherein said one or more amino acid substitution is at one or more position selected from the group of L234, L235, and P329 (Kabat EU index numbering).

Embodiment 37. The immunoconjugate of any one of Embodiments 25 to 36, wherein each subunit of the Fc domain comprises the amino acid substitutions L234A, L235A and P329G (Kabat EU index numbering).

Embodiment 38. The immunoconjugate of any one of Embodiments 16 to 37, comprising a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO:56.

Embodiment 39. The immunoconjugate of any one of Embodiments 16 to 37, comprising a polypeptide comprising the amino acid sequence of SEQ ID NO: 53, a polypeptide comprising the amino acid sequence of SEQ ID NO: 54, and a polypeptide comprising the amino acid sequence of SEQ ID NO:56.

Embodiment 40. The immunoconjugate of any one of Embodiments 16 to 37, comprising a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 53, a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO: 54, and a polypeptide comprising an amino acid sequence that is at least about 80%, at least about 85%, at least about 90%, at least about 95%, at least about 96%, at least about 97%, at least about 98%, at least about 99% or 100% identical to the sequence of SEQ ID NO:57.

Embodiment 41. The immunoconjugate of any one of Embodiments 16 to 37, comprising a polypeptide comprising the amino acid sequence of SEQ ID NO: 53, a polypeptide comprising the amino acid sequence of SEQ ID NO: 54, and a polypeptide comprising the amino acid sequence of SEQ ID NO:57.

Embodiment 42. The immunoconjugate of any one of Embodiments 16 to 41, essentially consisting of a mutant IL-7 polypeptide and an IgG1 immunoglobulin molecule, joined by a linker sequence.

Embodiment 43. One or more isolated polynucleotide encoding the mutant IL-7 polypeptide according to any one of Embodiments 1 to 14 or the immunoconjugate of any one of Embodiments 16 to 42.

Embodiment 44. One or more vector, particularly expression vector, comprising the polynucleotide(s) of Embodiment 43.

Embodiment 45. A host cell comprising the polynucleotide(s) of Embodiment 43 or the vector(s) of Embodiment 44.

Embodiment 46. A method of producing a mutant IL-7 polypeptide or an immunoconjugate comprising a mutant IL-7 polypeptide and an antibody that binds to PD-1, comprising (a) culturing the host cell of Embodiment 45 under conditions suitable for the expression of the mutant IL-7 polypeptide or the immunoconjugate, and optionally (b) recovering the mutant IL-7 polypeptide or the immunoconjugate.

Embodiment 47. A mutant IL-7 polypeptide or an immunoconjugate comprising a mutant IL-7 polypeptide and an antibody that binds to PD-1, produced by the method of Embodiment 46.

Embodiment 48. A pharmaceutical composition comprising the mutant IL-7 polypeptide of any one of Embodiments 1 to 14 and 47 or the immunoconjugate of any one of Embodiments 16 to 42 and 47 and a pharmaceutically acceptable carrier.

Embodiment 49. The mutant IL-7 polypeptide of any one of Embodiments 1 to 14 and 47 or the immunoconjugate of any one of Embodiments 16 to 42 and 47 for use as a medicament.

Embodiment 50. The mutant IL-7 polypeptide of any one of Embodiments 1 to 14 and 47 or the immunoconjugate of any one of Embodiments 16 to 42 and 47 for use in the treatment of a disease.

Embodiment 51. The mutant IL-7 polypeptide or the immunoconjugate for use in the treatment of a disease of Embodiment 50, wherein said disease is cancer.

Embodiment 52. Use of the mutant IL-7 polypeptide of any one of Embodiments 1 to 14 and 47 or the immunoconjugate of any one of Embodiments 16 to 42 and 47 in the manufacture of a medicament for the treatment of a disease.

Embodiment 53. The use of Embodiment 52, wherein said disease is cancer.

Embodiment 54. A method of treating a disease in an individual, comprising administering to said individual a therapeutically effective amount of a composition comprising the mutant IL-7 polypeptide of any one of Embodiments 1 to 14 and 47 or the immunoconjugate of any one of Embodiments 16 to 42 and 47 in a pharmaceutically acceptable form.

Embodiment 55. The method of Embodiment 54, wherein said disease is cancer.

Embodiment 56. A method of stimulating the immune system of an individual, comprising administering to said individual an effective amount of a composition comprising the mutant IL-7 polypeptide of any one of Embodiments 1 to 14 and 47 or the immunoconjugate of any one of Embodiments 16 to 42 and 47 in a pharmaceutically acceptable form.

### EXAMPLES

The following are examples of methods and compositions of the invention. It is understood that various other embodiments may be practiced, given the general description provided above.

### Assays Applied for Functional Characterization of PD1-IL7 Variants

### IL-7R Signaling Assay

In one aspect, an assay is provided to determine the potency and cis/trans-signaling of a PD-1-IL-7v immunoconjugate is provided. For this purpose, CD4 T cells from healthy donor PBMCs are sorted with CD4 beads (Miltenyi, #130-045-101) and activated for 3 days in presence of 1 μg/ml plate-bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells are harvested and washed several times to remove endogenous cytokines and half of the cells are labeled with Cell Trace Violet (CTV) (5 uM, 5 minutes at room temperature (RT); C34557, Thermo Scientific) and the other half are left unlabeled.

Then, the unlabelled cells are incubated with a saturating concentration of a competing anti-PD-1 antibody (in-house molecule, 10 μg/ml) for 30 minutes at RT followed by several washing steps to remove the excess unbound anti-PD-1 antibody. Thereafter, the PD-1 pre-blocked cells (25 μl, 6*106 cells/ml) are co-cultured 1:1 with the PD-1+ CTV-labeled cells (25 μl, 6×106 cells/ml) in a V-bottom plate before being treated for 12 minutes at 37° C. with increasing concentrations of treatment immunoconjugates (50 μl, 1:10 dilution steps). To preserve the phosphorylation state, an equal amount of Phosphoflow Fix Buffer I (100 μl, 557870, BD Bioscience) is added after 12 minutes incubation with the various constructs. The cells are then incubated for an additional 30 minutes at 37° C. before being permeabilized overnight at −80° C. with Phosphoflow PermBuffer III (558050, BD Bioscience). On the next day, STAT-5 in its phosphorylated form is stained for 30 minutes at 4° C. by using an anti-STAT-5P antibody (47/Stat5 (pY694) clone, 562076, BD Bioscience).

The cells are acquired at the fluorescence-activated cell sorting (FACS) BD-LSRFortessa or Symphony A3/A5 (BD Bioscience) instrument. The frequency of STAT-5P is determined with FLOWJO (V10) and plotted with GraphPad Prism (v8).

The dose-response curves on PD-1+ T cells provide information on the potency of the assessed molecules in signaling through the IL-7R. In addition, the dose-response curves on T cells pre-treated with a competing anti-PD-1 antibody, to prevent the PD-1 mediated delivery, show the potency of the molecules in providing IL-7R signaling independently from PD-1 expression.

### Suppression Assay

In one aspect, an assay is provided to assess whether PD-1-IL-7v immunoconjugates can reverse the regulatory T cell (Treg) suppression of conventional T cell (T_{conv}) effector functions. In some instances, T_{conv} and Treg are isolated and labeled.

In certain aspects, CD4+CD25+CD127dim Treg are isolated with the two-step Regulatory T cell Isolation Kit (Miltenyi, #130-094-775). In parallel, the CD4+CD25-T_{conv} are isolated by collecting the negative fraction of a CD25 positive selection (Miltenyi, #130-092-983) followed by a CD4+ enrichment (Miltenyi, #130-045-101). The T_{conv} are labeled with Carboxyfluoroscein succinimidyl ester (CFSE, eBioscience, #65-0850-84) and the T_{reg} are labeled with Cell Trace Violet (CTV, ThermoFisher scientific, C34557) to be able to distinguish them and to track the proliferation of both populations. T_{conv} and T_{reg} are then cultured together for 5 days, with or without treatment, in presence of CD4-CD25-PBMCs from an unrelated donor to provide an allospecific stimulation.

In certain aspects, on day 5, the accumulation of cytokines in the Golgi complex is enhanced by applying Protein Transport Inhibitors (GOLGIPLUG™ #555029, BD Bioscience; and GOLGISTOP™ #554724, BD Bioscience) for 5 hours prior to the FACS staining. The ability of the proliferated T_{conv} to secrete granzyme B (GrzB) in presence and absence of Treg is measured. Treg suppression is calculated with the following formula:

$$\% cytokine suppression = 100 - ( \frac{\% {cytokine}_{( Tconv + Treg \pm immunoconjugate )}}{\% {cytokine}_{( Tconv )}} * 100 )$$

Where % cytokine (T_{conv}+Treg±immunoconjugate) is the level of cytokine secreted by T_{conv} in the presence of Treg±treatment immunoconjugate and % cytokine (T_{conv}) is the level of cytokine secreted by T_{conv} in the absence of Treg. P is calculated using one-way ANOVA (*p<0.05, **p<0.01, ***p<0.001, ****p<0.0001).

### Internalization Assay

In one aspect, an assay is provided to assess the internalization of the different immunoconjugates. For this purpose PBMCs are isolated by density gradient centrifugation using Ficoll-Paque (Sigma-Aldrich). CD4 T cells are bead-sorted starting from 108 PBMCs by using a CD4-positive selection kit (Miltenyi, #130-045-101) following manufacturer instructions. CD4 T cells are then seeded in a 12 well plate previously coated with 1 μg/ml anti-CD3 (overnight pre-coated, clone OKT3, #317315, BioLegend) at 2-4×106 cells/well in RPMI 10% FBS in presence of 1 μg/ml of soluble anti-CD28 (clone CD28.2, #302923, BioLegend) and cultured for 3 days at 37° C.

Three days activated CD4 T cells are incubated in duplicates in FACS tubes in presence the immunoconjugates for 30 minutes at 4° C. The cells are then washed, divided in two groups, one of which is incubated for 3 additional hours at 37° C. and the other one is immediately stained with a PE labelled anti-PGLALA secondary antibody and anti-CD4 antibody (eBioscience) before being fixed with BD Cell Fix. After the 3 hours incubations, also the second group of the cells is stained with the PE labelled anti-PGLALA secondary antibody and anti-CD4 before fixation. The cells are then acquired at LSRFortessa (BD Biosciences) and data analysis are performed with FlowJo (V10) and plotted with GraphPad Prism (v8). The expression levels of detectable antibody on the cell surface at 4° C. is compared to the one at 37° C. to calculate the percentage of internalized molecules at 37° C. P is calculated using one-way ANOVA (*p<0.05, **p<0.01, ***p<0.001, ****p<0.0001).

### List of Molecules Used in Different Aspects of the Invention (Table 1)

TABLE 1
Concept IDs, components of PD1-IL7 fusion proteins and amino
acid sequences of described PD1-IL7 fusion proteins

| | PD1 binder | | | | SEQ ID |
|---|---|---|---|---|---|
| Tapir ID | ID | linker | IL7 variant | Description | NOs |
| P1AH4515 | FV000363 | (G4S)3 | wild-type | PD1-IL7wt_{—} | 1, 2, 3 |
| | | | | fully glycosylated | |
| P1AG3724 | FV000363 | (G4S)2G4 | G85E | PD1-IL7var21_{—} | 1, 2, 4 |
| | | | | fully glycosylated | |
| P1AG3725 | FV000363 | (G4S)2G4 | G85E, T93A, S118A | PD1-IL7var21_{—} | 1, 2, 5 |
| | | | | partially glycosylated | |
| | | | | (T93A/S118A) | |
| P1AG3727 | FV000363 | (G4S)2G4 | K81E, G85E, T93A, | PD1-IL7var18_21_{—} | 1, 2, 6 |
| | | | S118A | partially glycosylated | |
| | | | | (T93A/S118A) | |
| P1AH6010 | FV000363 | (G2SG2)3 | L77K, G85K | new PD1-IL7v_{—} | 1, 2, 7 |
| | | | | fully glycosylated | |
| P1AH6009 | FV000363 | (G2SG2)3 | L77K, Q136 | new PD1-IL7v_{—} | 1, 2, 8 |
| | | | | fully glycosylated | |
| P1AH6008 | FV000363 | (G2SG2)3 | V18K, Q136K | new PD1-IL7v_{—} | 1, 2, 9 |
| | | | | fully glycosylated | |
| P1AH6007 | FV000363 | (G2SG2)3 | V15K, Q136K | new PD1-IL7v_{—} | 1, 2, 10 |
| | | | | fully glycosylated | |
| P1AH6006 | FV000363 | (G2SG2)3 | K81A, Q136K | new PD1-IL7v_{—} | 1, 2, 11 |
| | | | | fully glycosylated | |
| P1AH6005 | FV000363 | (G2SG2)3 | D74K, G85K | new PD1-IL7v_{—} | 1, 2, 12 |
| | | | | fully glycosylated | |
| | | | | (PD1-IL7v2.2) | |
| P1AH6004 | FV000363 | (G2SG2)3 | V18K, G85K | new PD1-IL7v_{—} | 1, 2, 13 |
| | | | | fully glycosylated | |
| P1AH6003 | FV000363 | (G2SG2)3 | V15A, G85K | new PD1-IL7v_{—} | 1, 2, 14 |
| | | | | fully glycosylated | |
| P1AH6002 | FV000363 | (G2SG2)3 | V15A, K81A | new PD1-IL7v_{—} | 1, 2, 15 |
| | | | | fully glycosylated | |
| P1AH6017 | FV000363 | (G2SG2)3 | K81E, G85E, H152K | new PD1-IL7v_{—} | 1, 2, 16 |
| | | | | fully glycosylated | |
| P1AH6016 | FV000363 | (G2SG2)3 | K81E, G85E, E151K | new PD1-IL7v_{—} | 1, 2, 17 |
| | | | | fully glycosylated | |
| P1AH6047 | FV000363 | (G2SG2)3 | K81E, G85E, L125K | new PD1-IL7v_{—} | 1, 2, 18 |
| | | | | fully glycosylated | |
| P1AH6046 | FV000363 | (G2SG2)3 | K81E, G85E, L104Q, | new PD1-IL7v_{—} | 1, 2, 19 |
| | | | L113Q | fully glycosylated | |
| P1AH6045 | FV000363 | (G2SG2)3 | K81E, G85E, L113K | new PD1-IL7v_{—} | 1, 2, 20 |
| | | | | fully glycosylated | |
| P1AH6044 | FV000363 | (G2SG2)3 | K81E, G85E, L104K | new PD1-IL7v_{—} | 1, 2, 21 |
| | | | | fully glycosylated | |
| P1AH6043 | FV000363 | (G2SG2)3 | K81E, G85E, L80K | new PD1-IL7v_{—} | 1, 2, 22 |
| | | | | fully glycosylated | |
| P1AH6042 | FV000363 | (G2SG2)3 | K81E, G85E, L67K | new PD1-IL7v | 1, 2, 23 |
| | | | | fully glycosylated | |
| P1AH6041 | FV000363 | (G2SG2)3 | K81E, G85E, I30K | new PD1-IL7v_{—} | 1, 2, 24 |
| | | | | fully glycosylated | |
| P1AH6025 | FV000363 | (G2SG2)3 | L77K, G85E | new PD1-IL7v_{—} | 1, 2, 25 |
| | | | | fully glycosylated | |
| P1AH6021 | FV000363 | (G2SG2)3 | E13K, G85E | new PD1-IL7v_{—} | 1, 2, 26 |
| | | | | fully glycosylated | |
| | | | | (PD1-ILv2.6) | |
| P1AH6020 | FV000363 | (G2SG2)3 | K81Q, G85Q | new PD1-IL7v_{—} | 1, 2, 27 |
| | | | | fully glycosylated | |
| P1AH6019 | FV000363 | (G2SG2)3 | K81E, G85Q | new PD1-IL7v_{—} | 1, 2, 28 |
| | | | | fully glycosylated | |
| P1AH6018 | FV000363 | (G2SG2)3 | K81Q, G85E | new PD1-IL7v_{—} | 1, 2, 29 |
| | | | | fully glycosylated | |
| P1AH6039 | FV000363 | (G2SG2)3 | D8N, K10T, K81E, G85E, | new PD1-IL7v_{—} | 1, 2, 30 |
| | | | L113N, E115T | fully glycosylated | |
| P1AH6038 | FV000363 | (G2SG2)3 | D8N, K10T, K81E, G85E, | new PD1-IL7v_{—} | 1, 2, 31 |
| | | | K117G, S118T | fully glycosylated | |
| P1AH6037 | FV000363 | (G2SG2)3 | N37Q, E38N, N40T, | new PD1- | 1, 2, 32 |
| | | | K81E, G85E, T110N, | IL7v_additionally | |
| | | | S112T | glycosylated | |
| P1AH6036 | FV000363 | (G2SG2)3 | E38N, N40T, K81E, | new PD1- | 1, 2, 33 |
| | | | G85E, E106N, Q108T, | IL7v_additionally | |
| | | | P109S | glycosylated | |
| P1AH6035 | FV000363 | (G2SG2)3 | D8N, K10T, K81E, G85E, | new PD1- | 1, 2, 34 |
| | | | E52N, M54T | IL7v_additionally | |
| | | | | glycosylated | |
| P1AH6034 | FV000363 | (G2SG2)3 | D3N, E5T, E52N, M54T, | new PD1- | 1, 2, 35 |
| | | | K81E, G85E | IL7v_additionally | |
| | | | | glycosylated | |
| P1AH6033 | FV000363 | (G2SG2)3 | K81E, G85E, K150L, | new PD1- | 1, 2, 36 |
| | | | E151N, H152G | IL7v_additionally | |
| | | | | glycosylated | |
| P1AH6032 | FV000363 | (G2SG2)3 | K81E, G85E, L113N, | new PD1- | 1, 2, 37 |
| | | | E115T | IL7v_additionally | |
| | | | | glycosylated | |
| P1AH6031 | FV000363 | (G2SG2)3 | K81E, G85E, T110N, | new PD1- | 1, 2, 38 |
| | | | S112T | IL7v_additionally | |
| | | | | glycosylated | |
| P1AH6030 | FV000363 | (G2SG2)3 | K81E, G85E, E106N, | new PD1- | 1, 2, 39 |
| | | | Q108T, P109S | IL7v_additionally | |
| | | | | glycosylated | |
| P1AH6029 | FV000363 | (G2SG2)3 | E52N, M54T, K81E, G85E | new PD1- | 1, 2, 40 |
| | | | | IL7v_additionally | |
| | | | | glycosylated | |
| P1AH6028 | FV000363 | (G2SG2)3 | D48N, N50T, K81E, G85E | new PD1- | 1, 2, 41 |
| | | | | IL7v_additionally | |
| | | | | glycosylated | |
| P1AH6027 | FV000363 | (G2SG2)3 | N37Q, E38N, N40T, | new PD1- | 1, 2, 42 |
| | | | K81E, G85E | IL7v_additionally | |
| | | | | glycosylated | |
| P1AH6026 | FV000363 | (G2SG2)3 | E38N, N40T, K81E, G85E | new PD1- | 1, 2, 43 |
| | | | | IL7v_additionally | |
| | | | | glycosylated | |
| P1AH6024 | FV000363 | (G2SG2)3 | D3N, E5T, K81E, G85E | new PD1- | 1, 2, 44 |
| | | | | IL7v_additionally | |
| | | | | glycosylated | |
| P1AH6023 | FV000363 | (G2SG2)3 | D8N, K10T, K81E, G85E | new PD1- | 1, 2, 45 |
| | | | | IL7v_additionally | |
| | | | | glycosylated | |
| P1AH4506 | FV000363 | (G4S)3 | K81E, G85E | PD1-IL7var18_21 | 1, 2, 46 |
| P1AH4508 | FV000363 | (G4S)3 | wild-type | PD1-ILwt | 1, 2, 47 |
| P1AH4502 | FV000363 | (G4S)2G4 | wild-type | PD1-ILwt_fully | 1, 2, 48 |
| | | | | glycosylated | |
| P1AH4515 | FV000363 | (G2SG2)3 | wild-type | PD1-ILwt_fully | 1, 2, 49 |
| | | | | glycosylated | |
| P1AH4513 | FV000363 | (G2SG2)3 | K81E, G85E | PD1-IL7var18_21_{—} | 1, 2, 50 |
| | | | | fully glycosylated | |
| P1AG3048 | FV000363 | (G4S)2G4 | K81E, G85E | PD1-IL7var18_21_{—} | 1, 2, 51 |
| | | | | fully glycosylated | |
| P1AG0950 | FV000363 | 3G4S | K81E, G85E | PD1-IL7var18_21_{—} | 1, 2, 52 |
| | | | | fully glycosylated | |
| P1AJ0773 | FV020519 | (G2SG2)3 | K81E, G85E | PD1-IL7var18_21_{—} | 53, 54, 55 |
| | | | | fully glycosylated | |
| P1AI5585 | FV020519 | (G2SG2)3 | D74K, G85K | PD1-IL7v2.2 | 53, 54, 56 |
| | | | | fully glycosylated | |
| P1AI5587 | FV020519 | (G2SG2)3 | E13K, G85E | PD1-IL7v2.6 | 53, 54, 57 |
| | | | | fully glycosylated | |
| P1AF7303 | FV000359 | 3G4S | wild-type | Control: FAP (4B9) IgG | 85, 59, 60 |
| | | | | kh PGLALA huIL7wt | |
| P1AK9505 | FV012014 | 3G3SG | W142H | Reference - | 61, 62, 63 |
| | | | | OSE_FV012014_h- | |
| | | | | IL7(1-152)_W142H | |
| P1AL9335 | FV020519 | (G2SG2)3 | E13K, G85E | Murinized PD1-IL7v2.6 | 76, 77, 78 |

### Example 1

### Example 1.1 Design of IL7 Double Mutants

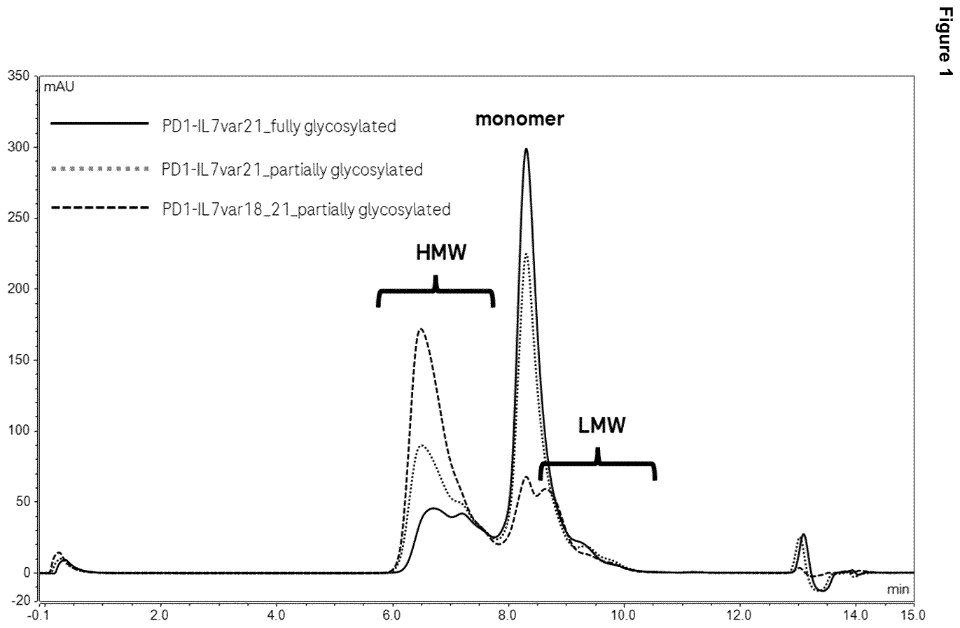

Single beneficial mutations in IL7, which result in diminished binding to the IL7R (described in WO 2021/209402), were combined to generate P1AG3727 (described in WO 2023/62050). This compound encompasses the two substitutions K81E and G85E and facilitates the desired MoA (cis-activation), as described in WO 2023/62050. The recombinant expression of P1AG3727 in stably transfected CHO cells resulted however in unexpected and undesired Chemistry, Manufacturing and Controls (CMC) properties, such as high protein aggregation (Table 2, FIG. 1).

TABLE 2
Titer and CMC characteristics after one-step ProteinA
purification in stable expression conditions.

| | | | Mono- | |
|---|---|---|---|---|
| | | HMW | mer | LMW |
| Tapir ID | IL7 variant | (%) | (%) | (%) |
| P1AG3724 | G85E (fully glycosylated) | 25.1 | 69.7 | 5.2 |
| P1AG3725 | G85E (partially | 41.4 | 53.4 | 5.3 |
| | glycosylarted_T93A/S118A) | | | |
| P1AG3727 | K81E/G85E- (partially- | 67.3 | 29 | 3.8 |
| | glycosylated: T93A/S118A) | | | |

The glycosylation status (fully glycosylated with all glycosylation sites present versus partially glycosylated with two N-glycosylation sites removed through the amino acid substitutions T93A and S118A) already has a negative impact on CMC properties. Moreover, the chosen combination of mutations (K81E and G85E) led to a further significant rise in HMW content and an unacceptable reduction in monomer content, for the manufacturing of a therapeutic molecule. This underscores that merely combining mutations does not invariably result in a molecule suitable for manufacturing. Consequently, novel fully glycosylated molecule variants were engineered with the objective to increase the protein monomer content.

Increased aggregation may be attributed to intrinsic biophysical properties, such as the isoelectric point (pI), which influences protein solubility at a specific pH. In P1AG3727, the introduction of an additional negative charge led to a reduction in the theoretical pH to 7.7 compared to the pI 8.0 of P1AG3724 (Table 3), which is close to the pH of the endoplasmic reticulum (pH of 7.2+/−0.3), or to unfavorable intramolecular charge distribution favoring aggregation or to minor structural changes favoring aggregation.

TABLE 3
Theoretical pI of IL7 variants. pI determined
based on amino acid composition.

| Tapir ID | IL7 variant | Theoretical pI |
|---|---|---|
| P1AH4515 | wild-type - fully glycosylated | 8.26 |
| P1AG3724 | G85E - fully glycosylated | 8.00 |
| P1AG3725 | G85E - partially glycosylated | 8.00 |
| | (T93A/S118A) | |
| P1AG3727 | K81E/G85E - partially glycosylated | 7.7 |
| | (T93A/S118A) | |

New designs to potentially decrease aggregation propensity were based on categories A to E (Table 4).

TABLE 4
Categories of new designs

| | |
|---|---|
| A | (Tapir IDs P1AH6002 to P1AH6010): includes novel combinations of substitutions the single |
| | beneficial mutations previously identified, as described in WO 2021/209402. The combinations of |
| | substitutions were selected based on their reduced binding affinity to the IL7 receptor (faster |
| | dissociation) and in vitro cis-activation potential, as described in WO 2021/209402. |
| B | (Tapir IDs P1AH6041 to P1AH6047, P1AH6016, P1AH6017): includes pI compensating alkaline |
| | residues in addition to the two amino acid changes in P1AH4513 (K81E/G85E), which represents |
| | a fully glycosylated form of P1AG3727. |
| C | (Tapir IDs P1AH6018 to P1AH6021, P1AH6025): includes alternative amino acid replacements |
| | at residue 81 and/or 85 or additional replacements, while keeping on of the two residues diverging |
| | in P1AH4513 from the IL7 wild-type sequence (K81E/G85E). |
| D | (Tapir IDs P1AH602, P1AH6024, P1AH6026 to P1AH6039): includes introduction of additional |
| | N-glycosylation sites and thus potentially increase the content of sialylated N-glycans to enhance |
| | protein solubility, by increasing protein hydrophilicity due to the negative charge and the polar |
| | nature of sialic acids. |
| E | (P1AH4502, P1AH4508, P1AH4506): includes variation of linker composition to avoid |
| | conformational constraints leading to improper folding and thus potentially increased aggregation. |

### Example 1.2 Production and Analytics of PD1-IL7v Fusion Proteins in HEK Cells

The plasmids for antibody expression were generated by gene synthesis (Twist Bioscience, USA) and cloned into Roche's in-house expression vector using standard cloning procedures. Antibodies were expressed by transient transfection of Expi293FTM cells (ThermoFisher scientific, USA). Cells were seeded in Expi293TM medium (Gibco, Cat. N° 1435101) at a density of 3×106/ml. Expression vectors and ExpiFectamine (Gibco, ExpiFectamine™ transfection kit, Catalog number 13385544) were separately mixed in OptiMEM™ reduced serum medium (Gibco, Catalog number 11520386). After 1 minute, both solutions were combined, mixed by pipetting and incubated for 7 minutes at room temperature. Cells were added to the expression vector/ExpiFectamine solution and incubated for 24 hours at 37° C. in a shaking incubator with a 5% CO2 atmosphere. One day post transfection, supplements (Transfection Enhancers 1 and 2, ExpiFectamine™ transfection kit) were added. Cell supernatants were harvested after 6-7 days by centrifugation and subsequent filtration (1.2 μm filter).

Antibodies were purified from cell culture supernatants by affinity chromatography using MabSelectSure-Sepharose™ (GE Healthcare, Sweden) chromatography. Purification was performed with Roboclumns® (Repligen, USA) on a Tecan Evo freedom liquid handling system. Briefly, sterile filtered cell culture supernatants were captured on a MabSelect SuRe resin equilibrated with PBS buffer (10 mM Na2HPO4, 1 mM KH2PO4, 137 mM NaCl and 2.7 mM KCl, pH 7.4), washed with equilibration buffer and eluted with 25 mM citrate, pH 3.0. After neutralization with 1 M Tris pH 9.0, aggregated protein was separated from monomeric antibody species by size exclusion chromatography (Superdex 200, GE Healthcare) in 20 mM histidine, 140 mM NaCl, pH 6.0. Monomeric protein fractions were pooled and stored at −80° C. Product quality was analysed by CE-SDS using LabChip® HT Protein Express Chip and LabChip® GXII protein characterization system (Perkin Elmer) applying the standard protocol for proteins.

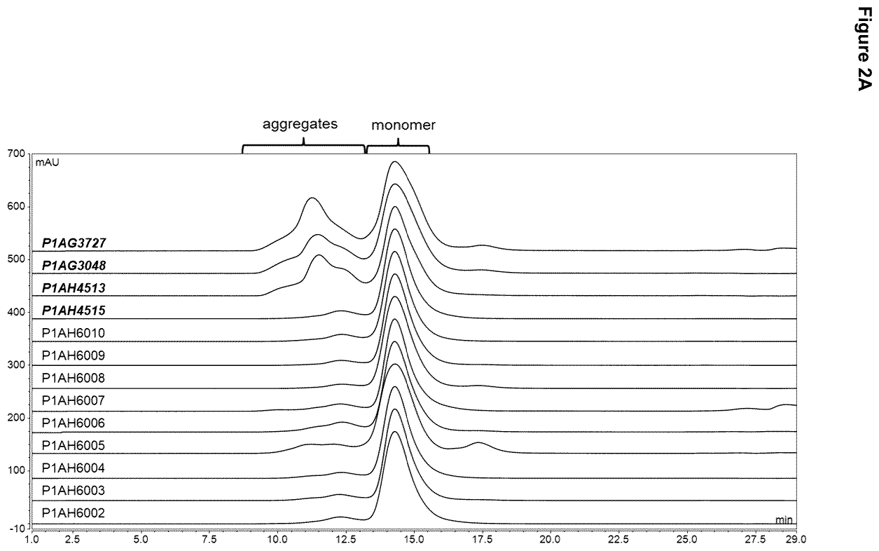

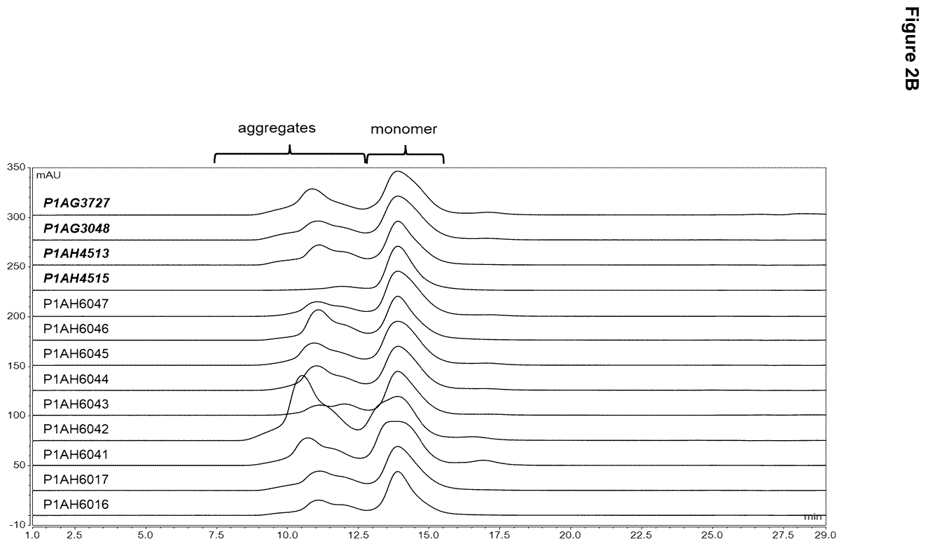

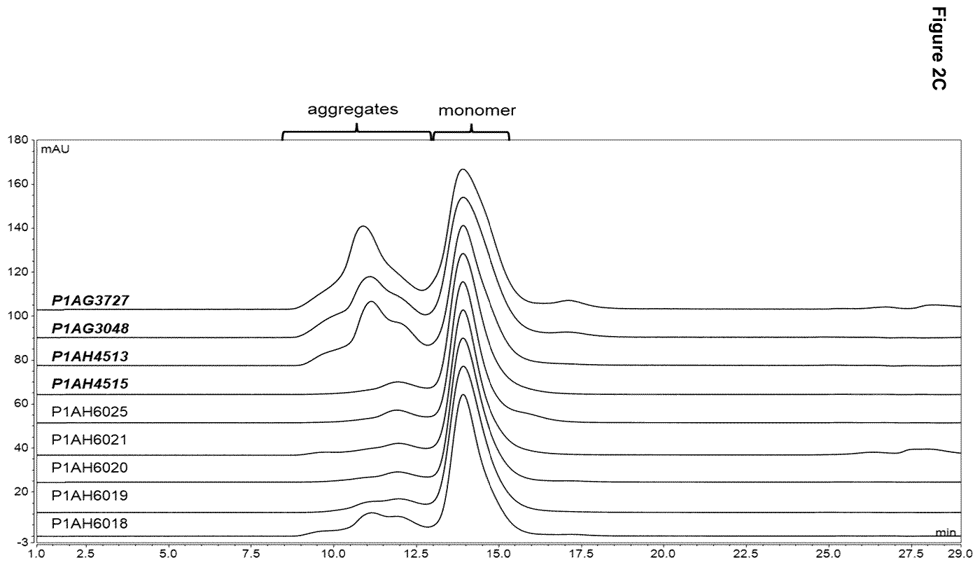

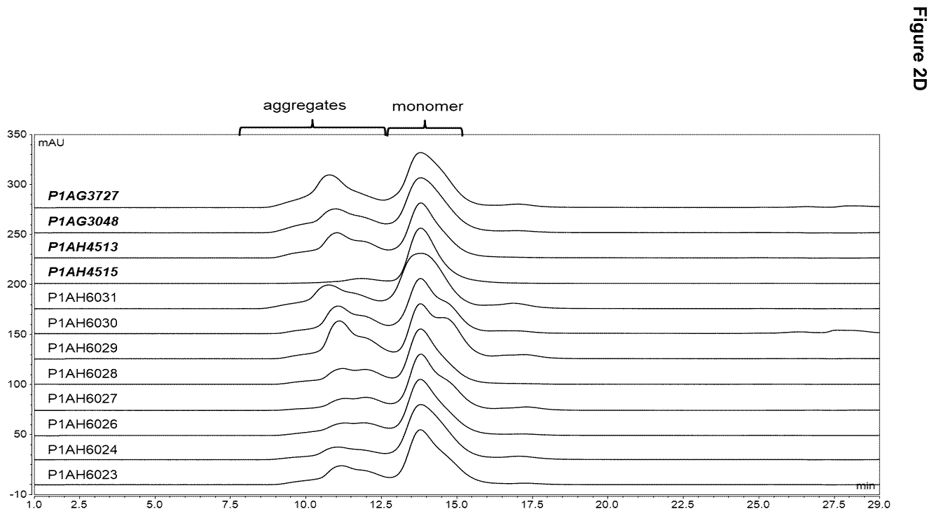

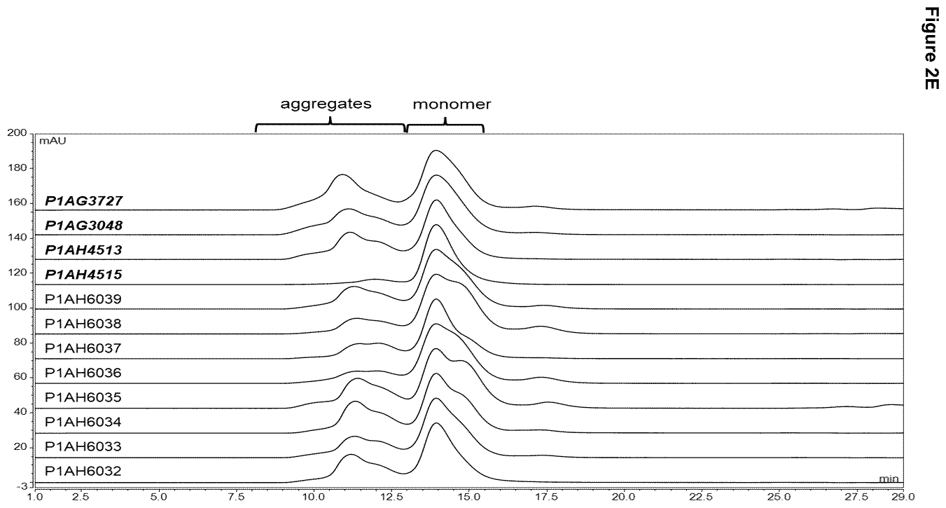

Table 5 and FIGS. 2A-F show the expression and product quality results of the evaluated PD1-IL7 variants. The product titer of the different variants ranged between 144 and 433 μg/ml (Table 5). PD1-IL7 wt fusion (P1AH4515) had a titer of 186 μg/ml, while P1AH4513, which is targeted for improvement, had a titer of 144 μg/ml. Quality analysis of the SEC-purified material revealed that the HMW content after Protein A chromatography differed significantly between 4.5 and 56% for the different IL7 variants, whereas P1AH4515 showed 7.8% and P1AH4513 showed 39% HMW content. The monomer content after Protein A chromatography differed between 41 and 95%, with P1AH4515 holding 92% and P1AH4513 holding 62% monomeric fraction. No correlation between titer and monomer or HMW content was found.

TABLE 5
Design category, titer and CMC characteristics after one-step ProteinA purification.

| | | | Main Peak | | | | |
|---|---|---|---|---|---|---|---|
| | | | [%] | | Monomer | | Selected |
| | | Titer | (by non- red | HMWs [%] | [%] | LMWs [%] | for in vitro |
| Tapir ID | Category | (ug/ml) | CE-SDS) | (by SEC) | (by SEC) | (by SEC) | testing |
| P1AH6010 | A | 193 | 81.6 | 8.0 | 92.0 | 0.0 | x |
| P1AH6009 | A | 213 | 67.2 | 4.6 | 95.4 | 0.0 | x |
| P1AH6008 | A | 217 | 55.9 | 4.5 | 92.6 | 2.9 | x |
| P1AH6007 | A | 197 | 84.0 | 10.7 | 89.4 | 0.0 | x |
| P1AH6006 | A | 201 | 78.3 | 11.5 | 87.6 | 1.0 | x |
| P1AH6005 | A | 217 | 60.2 | 12.5 | 78.7 | 8.9 | x |
| P1AH6004 | A | 433 | 73.3 | 7.6 | 92.5 | 0.0 | x |
| P1AH6003 | A | 362 | 76.1 | 7.5 | 92.5 | 0.0 | x |
| P1AH6002 | A | 388 | 72.3 | 7.1 | 92.9 | 0.0 | x |
| P1AH6017 | B | 211 | 83.9 | 36.7 | 63.3 | 0.0 | |
| P1AH6016 | B | 183 | 88.5 | 34.0 | 66.0 | 0.0 | |
| P1AH6047 | B | 342 | 75.9 | 27.9 | 70.0 | 2.1 | |
| P1AH6046 | B | 225 | 85.3 | 43.0 | 57.0 | 0.0 | |
| P1AH6045 | B | 295 | 69.4 | 34.8 | 62.5 | 2.7 | |
| P1AH6044 | B | 244 | 72.0 | 37.6 | 59.7 | 2.7 | |
| P1AH6043 | B | 315 | 80.0 | 26.0 | 72.0 | 1.9 | |
| P1AH6042 | B | 228 | 41.6 | 56.1 | 41.0 | 2.9 | |
| P1AH6041 | B | 298 | 54.7 | 36.1 | 58.5 | 5.5 | |
| P1AH6025 | C | 191 | 79.8 | 8.6 | 84.3 | 7.1 | x |
| P1AH6021 | C | 184 | 81.2 | 12.0 | 87.9 | 0.0 | x |
| P1AH6020 | C | 207 | 78.5 | 9.0 | 91.0 | 0.0 | x |
| P1AH6019 | C | 187 | 86.4 | 12.4 | 87.6 | 0.0 | x |
| P1AH6018 | C | 200 | 88.7 | 22.5 | 77.5 | 0.0 | x |
| P1AH6039 | D | 309 | 71.3 | 28.2 | 68.4 | 3.4 | |
| P1AH6038 | D | 256 | 60.8 | 19.0 | 73.4 | 7.6 | |
| P1AH6037 | D | 263 | 84.0 | 27.0 | 71.7 | 1.4 | |
| P1AH6036 | D | 290 | 66.0 | 19.1 | 75.1 | 5.7 | |
| P1AH6035 | D | 28 | 93.8 | 31.1 | 64.1 | 4.8 | |
| P1AH6034 | D | 278 | 67.3 | 35.3 | 62.4 | 2.3 | |
| P1AH6033 | D | 254 | 74.8 | 30.1 | 67.0 | 2.9 | |
| P1AH6032 | D | 180 | 83.1 | 37.0 | 63.0 | 0.0 | |
| P1AH6031 | D | 229 | 58.7 | 30.0 | 64.8 | 5.2 | |
| P1AH6030 | D | 225 | 70.8 | 34.7 | 62.7 | 2.7 | |
| P1AH6029 | D | 230 | 94.1 | 38.0 | 58.3 | 3.8 | |
| P1AH6028 | D | 231 | 83.9 | 30.5 | 69.5 | 0.0 | |
| P1AH6027 | D | 261 | 48.4 | 23.1 | 72.9 | 4.0 | |
| P1AH6026 | D | 258 | 82.9 | 25.3 | 73.3 | 1.4 | |
| P1AH6024 | D | 205 | 69.2 | 21.4 | 74.6 | 3.9 | |
| P1AH6023 | D | 233 | 81.4 | 30.4 | 65.7 | 3.9 | |
| P1AH4506 | E | 177 | 74.8 | 33.1 | 64.4 | 2.5 | |
| P1AH4508 | E | 180 | 77.0 | 9.1 | 90.9 | 0.0 | |
| P1AH4502 | E | 170 | 74.4 | 8.2 | 91.8 | 0.0 | |
| P1AH4515 | control | 186 | 80.7 | 7.8 | 92.2 | 0.0 | |
| P1AH4513 | control | 144 | 84.5 | 38.5 | 61.7 | 0.0 | |
| P1AG3727 | control | 156 | 67.8 | 37.8 | 59.4 | 2.9 | |
| P1AG3048 | control | 146 | 76.8 | 35.9 | 62.2 | 1.9 | |

Variants with improved monomer content over P1AH4513 (61.7%) were selected for subsequent in vitro studies. Only variants with a monomer content of at least 75% were moved forward. The selected variants are highlighted in Table 5.

Out of the fortytwo newly designed variants, fourteen variants (highlighted as selected in Table 5) demonstrated a significant improvement compared to P1AG3048 and P1AH4513 with enhanced monomer content after Protein A purification (defined as >75% monomer content). Nine of the selected variants were designed by combining pre-selected single mutants (WO 2023/62050) that showed reduced binding affinity as well as significantly increased dissociation rates (kd) to the IL7R (category A). The remaining five selected variants were designed based on P1AH4513, where alternative amino acids were introduced at positions 81 and/or 85. These positions exhibit divergence in P1AH4513 from the wild-type IL7 sequence (category C). None of the variants possessing additional stabilizing mutations or additional glycosides, nor variants with different linker length demonstrated an enhancement in monomer content (category B, D and E) thus having no impact on CMC properties. Given that P1AH4513 does not meet the required CMC properties, the straightforward combination of advantageous single mutations as described in WO 2023/62050 did not lead to a molecule that could be efficiently produced. Rational design, incorporating multiple design approaches (A to E), in combination with a broad screening of variants were needed to identify a variant that not only exhibits the desired MoA, but also possesses acceptable CMC properties.

### Example 1.3 Selection of IL-7 Variants Through PD-1 Mediated Cis Delivery of IL-7R Signaling (STAT-5P) to PD-1⁺ CD4 T Cells

In this experiment, the STAT5 phosphorylation was used as a readout to assess whether PD1-IL7v would signal through the IL-7R upon binding to PD-1 on the same CD4 T cells (cis) expressing PD-1, rather than on neighbor CD4 T cells (trans) devoided of PD-1 on their surface. For this purpose, CD4 T cells from healthy donor PBMCs are sorted with CD4 beads (Miltenyi, #130-045-101) and activated for 3 days in presence of 1 μg/ml plate-bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells are harvested and washed several times to remove endogenous cytokines and half of the cells are labeled with Cell Trace Violet (CTV) (5 μM, 5 minutes at room temperature (RT); C34557, Thermo Scientific) and the other half are left unlabeled.

Then, the unlabelled cells are incubated with a saturating concentration of a competing anti-PD-1 antibody (in-house molecule, 10 μg/ml) for 30 minutes at RT, in order to prevent the binding to PD-1 by the PD1-IL7 variants, followed by several washing steps to remove the excess unbound anti-PD-1 antibody. Thereafter, the PD-1 pre-blocked cells (25 μl, 6*106 cells/ml) are co-cultured 1:1 with the PD-1+ CTV-labeled cells (25 μl, 6×106 cells/ml) in a V-bottom plate before being treated for 12 minutes at 37° C. with increasing concentrations of treatment immunoconjugates (50 μl, 1:10 dilution steps). To preserve the phosphorylation state, an equal amount of Phosphoflow Fix Buffer I (100 μl, 557870, BD Bioscience) is added after 12 minutes incubation with the various constructs. The cells are then incubated for an additional 30 minutes at 37° C. before being permeabilized overnight at −80° C. with Phosphoflow PermBuffer III (558050, BD Bioscience). On the next day, STAT-5 in its phosphorylated form is stained for 30 minutes at 4° C. by using an anti-STAT-5P antibody (47/Stat5 (pY694) clone, 562076, BD Bioscience).

The cells are acquired at the fluorescence-activated cell sorting (FACS) BD-LSRFortessa or Symphony A3/A5 (BD Bioscience) instrument. The frequency of STAT-5P is determined with FLOWJO (V10) and plotted with GraphPad Prism (v8).

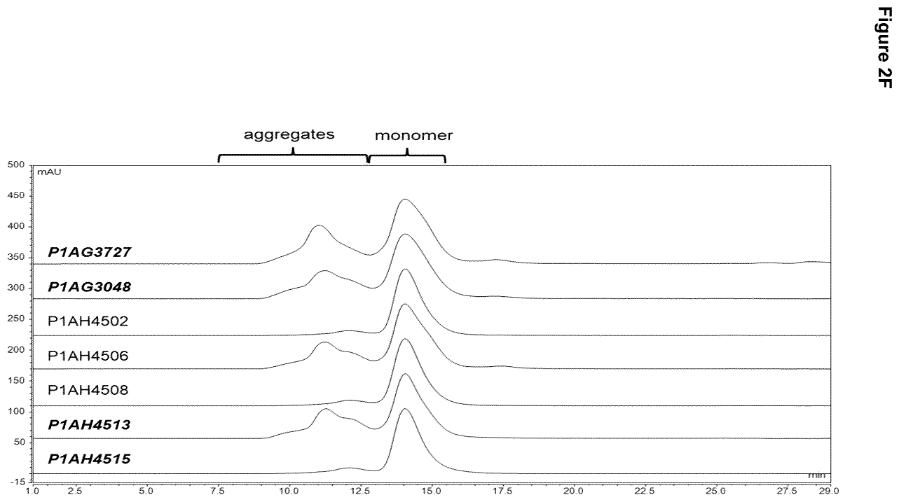

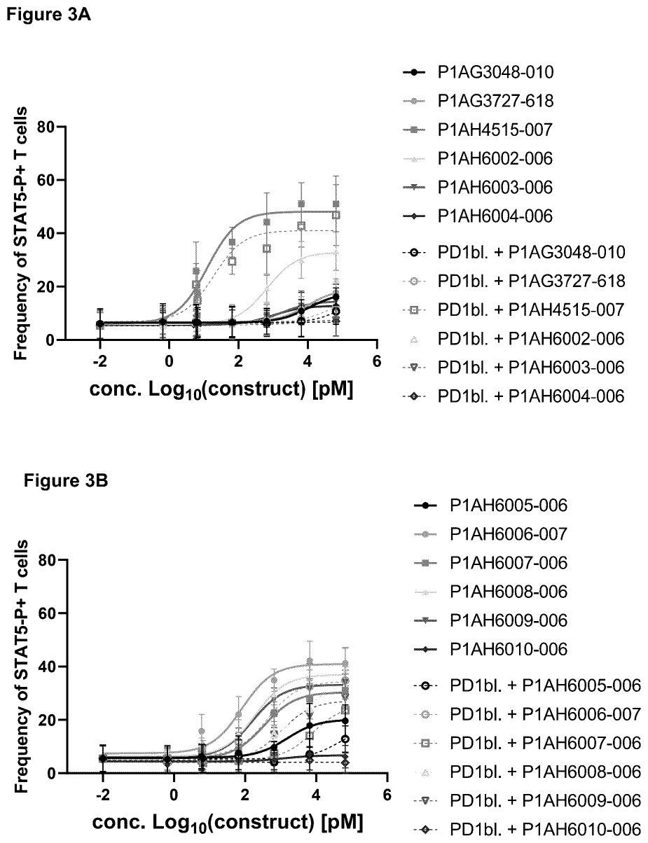

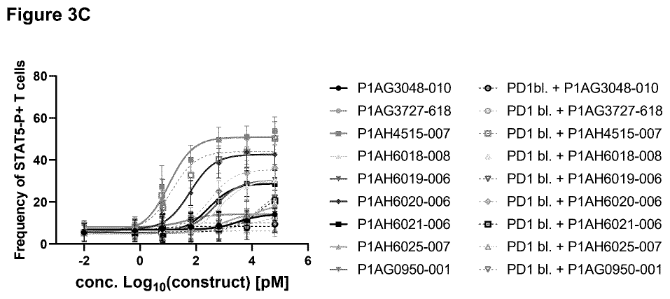

FIG. 3A and FIG. 3C show the frequency of STAT-5P+ cells within the PD1+ T cells, labelled with Cell Trace Violet and those labelled with CFSE pre-blocked with PD-1 antibody, upon exposure to 0.1 ug/ml of the 14 PD1-IL7 variants. The data in FIG. 3A-C show the potency difference of novel double mutant PD1-IL7 variants in PD-1+ and PD-1-(pre-blocked cells with competing anti-PD-1 antibody) CD4 T cells. The potency measurement on the PD1+CD4 T cells reflects the PD1-mediated delivery of IL-7 versus the PD1-independent delivery of IL-7R agonism in PD1-CD4 T cells. The STAT-5P EC50 fold increase between PD1-mediated and PD-1 independent delivery of IL-7R agonsim of each PD1-IL7v molecule was calculated by dividing the EC50 of the PD-1− CD4 T cells by the EC50 of PD1+ T cells. This provides evidence on the strength of the cis-delivery, PD1-mediated, of IL7v for each of the IL-7 double mutants. Furthermore, the EC50 fold increase between the PD1-IL7v and PD1-IL7 wt was calculated by dividing the EC50 of PD1-IL7v by the EC50 of PD1-IL7 wt. This indicated the loss in potency of the PD1-IL 7v due to the reduced affinity to the IL-7Rα. Tables 6, 7 and 8 show the EC50, EC50 fold increase (cis-delivery and potency reduction) and Area under the Curve of the dose-response STAT-5 phosphorylation for the selected mutants and PD1-IL7 wt on PD-1+ and PD-1− CD4 T cells obtained from 5 donors.

In summary, of the the tested PD1-IL7 variants only P1AH6002, P1AH6005, P1AH6007, P1AH6018, P1AH6019 and P1AH6021 show the contribution of the PD-1 in mediating the delivery of the IL-7 variants, in a dose response manner, to the IL-7R on PD-1 expressing versus PD-1− CD4 T cells with a minimum cis-activity window of 20 fold and a potency reduction of at least 25 fold compared to PD1-IL7 wt (FIG. 3A-C and Tables 6, 7 and 8).

TABLE 6
Cis/trans activity of new IL7 double mutant variants

| | | | Cis-delivery: | | | | |
|---|---|---|---|---|---|---|---|
| | | | fold increase in | | | | |
| | PD1+ | PD1blocked | EC50 | Potency reduction | PD1+ | PD1bl. | |
| | (EC50) | (EC50) | EC50 [with | to PD1-IL7wt: | (AUC) | (AUC) | |
| | (Mean ± | (Mean ± | PD1bl]/EC50 | EC50 (PD1+)/ | (Mean ± | (Mean ± | Delta |
| Tapir-ID | SEM) | SEM) | [no PD1bl | EC50 (PD1-IL7wt) | SEM) | SEM) | AUC |
| P1AG3048- | 11024 | 136791 | 12.4 | 874.2 | 13.1 | 6.1 | 7 |
| 010 | | | | | | | |
| P1AG3727- | 10022 | 38325 | 3.8 | 794.8 | 15.3 | 7.8 | 7.4 |
| 618 | | | | | | | |
| P1AH4515 | 12.6 | 19.2 | 1.5 | 1 | 157.1 | 125.1 | 32 |
| P1AH6002- | 663.6 | 21030 | 31.7 | 52.6 | 57.1 | 16.7 | 40.4 |
| 006 | | | | | | | |
| P1AH6003- | 1767 | 7188 | 4.1 | 140.1 | 16.6 | 4.2 | 12.4 |
| 006 | | | | | | | |
| P1AH6004- | 1011 | 100.5 | 0.1 | 80.2 | 14.1 | 3.4 | 10.6 |
| 006 | | | | | | | |

TABLE 7
Cis/trans activity of new IL7 double mutant variants

| | | | Cis-delivery: | | | | |
|---|---|---|---|---|---|---|---|
| | | | fold increase in | | | | |
| | PD1+ | PD1blocked | EC50 | Potency reduction | PD1+ | PD1bl. | |
| | (EC50) | (EC50) | EC50 [with | to PD1-IL7wt: | (AUC) | (AUC) | |
| | (Mean ± | (Mean ± | PD1bl]/EC50 | EC50 (PD1+)/ | (Mean ± | (Mean ± | Delta |
| Tapir-ID | SEM) | SEM) | [no PD1bl | EC50 (PD1-IL7wt) | SEM) | SEM) | AUC |
| P1AH6005- | 1499 | 47136 | 31.4 | 118.9 | 26.5 | 6.9 | 19.5 |
| 006 | | | | | | | |
| P1AH6006- | 81.4 | 423.1 | 5.2 | 6.5 | 107.8 | 67.4 | 40.4 |
| 007 | | | | | | | |
| P1AH6007- | 345.4 | 8200 | 23.7 | 27.4 | 56.1 | 21.3 | 34.9 |
| 006 | | | | | | | |
| P1AH6008- | 186.6 | 1624 | 8.7 | 14.8 | 82.5 | 43.3 | 39.2 |
| 006 | | | | | | | |
| P1AH6009- | 160.3 | 1078 | 6.7 | 12.7 | 73 | 38.9 | 34 |
| 006 | | | | | | | |
| P1AH6010- | 1169 | n.d. | n.d. | 92.7 | 7.1 | 7.8 | −0.7 |
| 006 | | | | | | | |

TABLE 8
Cis/trans activity of new IL7 double mutant variants

| | | | Cis-delivery: | Potency | | | |
|---|---|---|---|---|---|---|---|
| | | | fold increase in | reduction to | | | |
| | PD1+ | PD1blocked | EC50 | PD1-IL7wt: | PD1+ | PD1bl. | |
| | (EC50) | (EC50) | EC50 [with | EC50 (PD1+)/ | (AUC) | (AUC) | |
| | (Mean ± | (Mean ± | PD1bl]/EC50 [no | EC50 (PD1- | (Mean ± | (Mean ± | Delta |
| Tapir-ID | SEM) | SEM) | PD1bl | IL7wt) | SEM) | SEM) | AUC |
| P1AG3048- | 3232 | 3.2 | 0.001 | 299.3 | 19.2 | 12.3 | 7 |
| 010 | | | | | | | |
| P1AG3727- | 6753 | 13651 | 2 | 625.3 | 22 | 15.1 | 6.9 |
| 618 | | | | | | | |
| P1AH4515 | 10.8 | 13.7 | 1.3 | 1 | 173.3 | 142.8 | 30.5 |
| P1AH6018- | 766.3 | 27928 | 36.4 | 71 | 53 | 17.6 | 35.5 |
| 008 | | | | | | | |
| P1AH6019- | 496.4 | 17429 | 35.1 | 46 | 58 | 21.5 | 36.5 |
| 006 | | | | | | | |
| P1AH6020- | 62 | 317.5 | 5.1 | 5.7 | 118.2 | 75.8 | 42.4 |
| 006 | | | | | | | |
| P1AH6021- | 286.4 | 15026 | 52.5 | 26.5 | 58.3 | 17.4 | 41 |
| 006 | | | | | | | |
| P1AH6025- | 840.2 | 8.6 | 0.01 | 77.8 | 19.9 | 4.8 | 15.1 |
| 007 | | | | | | | |
| P1AG0950- | 44.6 | 7.4 | 0.2 | 4.1 | 19.5 | 7.8 | 11.7 |
| 001 | | | | | | | |

### Example 1.4 Further Selection of PD1-IL7 Variants Based on the Ability to Rescue T_{conv} Effector Function from T_{Reg} Suppression

In this experiment, it was assessed whether the PD1-IL7 mutants can reverse the T_{reg} suppression of T_{conv} effector functions. Therefore, a suppressive-function assay was established, and for this purpose, T_{conv} and T_{reg} were isolated and labeled as follow. CD4⁺ CD25⁺ CD127dim Regulatory T cells (T_{reg}) were isolated with the two-step Regulatory T cell Isolation Kit (Miltenyi, #130-094-775). In parallel the CD4⁺ CD25⁻ conventional T cells (T_{conv}) were isolated by collecting the negative fraction of a CD25 positive selection (Miltenyi, #130-092-983) followed by a CD4+ enrichment (Miltenyi, #130-045-101). The T_{conv} were labeled with CFSE (eBioscience, #65-0850-84) and the T_{reg} were labeled with Cell Trace Violet (ThermoFisher scientific, C34557) to track the proliferation of both populations. T_{conv} and T_{reg} were then cultured together for 5 days with CD4-CD25-PBMCs from an unrelated donor to provide an allospecific stimulation in presence or absence of 6 nM of treatment. On day 5, the accumulation of cytokines in the Golgi complex was enhanced by applying Protein Transport Inhibitors (GolgiPlug #555029, BD and GolgiStop #554724, BD) for 5 hours prior to the FACS staining.

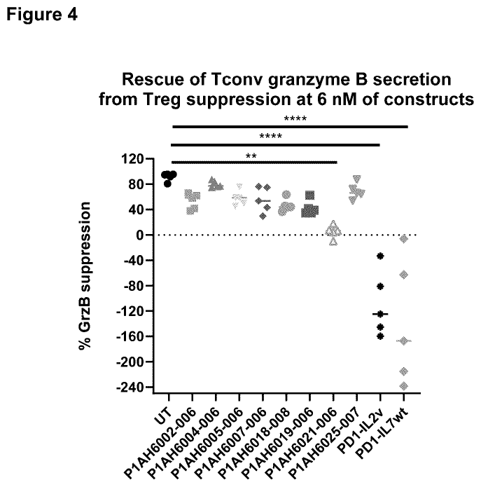

The ability of the proliferated T_{conv} to secrete granzyme B (GrzB; FIG. 4) in presence and absence of T_{reg} was measured. The T_{reg} suppression was calculated with the following formula:

$$\% cytokine suppression = 100 - ( \frac{\% {cytokine}_{( Tconv + Treg \pm immunoconjugate )}}{\% {cytokine}_{( Tconv )}} * 100 )$$

Where % cytokine (_{Tconv+Treg±immunoconjugate}) is the level of cytokine secreted by T_{conv} in the presence of T_{reg}±treatment immunoconjugate and % cytokine (T_{conv}) is the level of cytokine secreted by T_{conv} in the absence of T_{reg}. In FIG. 4, each symbol represents a separate donor, horizontal lines indicate medians with N=5 donors, from 3 independent experiments. P was calculated using one-way ANOVA (*p<0.05, **p<0.01, ***p<0.001, ****p<0.0001).

FIG. 4 shows the T_{reg} mediated suppression of granzyme B production by T_{conv} in a 5 days coculture in presence or absence of selected PD1-IL7 variants. Table 9 shows the percentage of T_{reg}-mediated suppression of granzyme B production by T conv and the percentage of rescued granzyme B secretion by T_{conv} from T_{reg} suppression in presence of 6 nM of the different immunoconjugates. The data were obtained with T cells from five different donors.

In untreated samples, 91.6% of granzyme B secretion by T_{conv} was suppressed by T_{reg}. PD1-IL2v was used as positive control due to its ability to overcome T_{reg} suppression which in this case was reduced to −108.9% resulting in the ability to rescue T_{conv} granzyme B production to 208.9%. The T_{reg} suppression was even further reduced to 138% by PD1-IL7 wt, corresponding to 238% of rescued granzyme B production. Interestingly, the PD1-IL7v mutants resulted in a reduction of T_{reg} suppression ranging between 79% to 5.3% depending on the decreased affinity of the mutated IL-7 variants for the IL-7R. These results reveal those mutants with a reduced affinity for the IL-7R which still maintain the IL-7 wt competitive advantage for T_{conv} versus T_{reg} (FIG. 4 and Table 9).

TABLE 9
Percentage of T_{reg}-mediated suppression of granzyme
B produced by T_{conv} and percentage of rescued granzyme
B production by T conv. Median of 5 donors.

| | | Percentage of | Percentage of |
|---|---|---|---|
| | | suppressed | rescued |
| | Concen- | Granzyme B | Granzyme B |
| | tration | production by | production by |
| Tapir-ID | (nM) | T_{conv} | T_{conv} |
| UT | | 91.6 | 8.4 |
| P1AH6002-006 | 6 | 53.2 | 46.8 |
| P1AH6004-006 | 6 | 79.9 | 20.1 |
| P1AH6005-006 | 6 | 57.9 | 42.1 |
| P1AH6007-006 | 6 | 55.4 | 44.6 |
| P1AH6018-008 | 6 | 46.7 | 53.3 |
| P1AH6019-006 | 6 | 42.3 | 57.7 |
| P1AH6021-006 | 6 | 5.3 | 94.7 |
| P1AH6025-007 | 6 | 68.8 | 31.2 |
| PD1-IL2v | 6 | −108.9 | 208.9 |
| (P1AE4422) | | | |
| PD1-IL7wt | 6 | −137.9 | 237.9 |
| (P1AH4515) | | | |

### Example 2

### Example 2.1. Impact of the Combination of the PD1 Binder and IL7 Variant on Titer and Monomer Content

The PD1 binder and the IL7 variant were individually optimized, focusing on immunological properties for the PD1 binder and CMC properties for the IL7 variant (as mentioned above). To evaluate the effects of the two PD1 binder, FV000363 (WO2017/055443A1, WO 2018/184964A1) and a de-risked variant FV020519, in combination with the IL7 variants, combinations thereof were produced transiently in CHO cells and subsequently analyzed regarding properties like titer and monomer content.

### Example 2.2 Transient Production of IgG-Like Proteins in CHO Cells

PD1-IL7v constructs were prepared in a 750 ml scale by Evitria using their proprietary vector system with conventional (non-PCR based) cloning techniques and using suspension-adapted CHO cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). The supernatant was harvested by centrifugation and subsequent filtration (0.2 μm filter). Proteins were purified from filtered cell culture supernatants on a liquid handling platform in 96 well format using a one-step Protein A affinity chromatography. In brief, supernatants were loaded on ProPlus Phy Tip Columns (MabSelect SuRe™, Phynexus) and washed with a buffer comprising sodium phosphate, sodium citrate. Target proteins were eluted in a low pH buffer and neutralized thereafter. The Titer was determined by Bio-Layer Interferometry (BLI) measurements on the Octet N1 (Sartorius). The sensors used are the Octet ProA Biosensors (Sartorius). Purity and molecular weight of the micro-purified proteins were analyzed by CE-SDS in the presence and absence of a reducing agent using a LabChipGXII or LabChip GX Touch, Perkin Elmer (Caliper). Determination of the aggregate content was performed by HPLC chromatography at 25° C. using analytical size-exclusion column (TSKgel G3000 SW XL or UP-SW3000) equilibrated in a running buffer comprising KH2PO4, KCl, and NaN3.

The IL7 variant is the main determinant of the construct's critical performance metrics, including titer, HMW and monomer content. In contrast, the selection of the PD1-binder (FV000363 vs. FV020519) has a marginal effect on the CMC characteristics, meaning switch of binder does not impact CMC properties (Table10).

TABLE 10
Titer and CMC characteristics of PD1-IL7 fusion proteins.

| | aSEC | aSEC | | CE-SDS (non- | |
|---|---|---|---|---|---|
| | % | % | aSEC | reduced) | Titer |
| Tapir ID | HMW | monomer | % LMW | % purity | mg/L |
| P1AI5587 | 10.24 | 79.01 | 10.75 | 84.48 | 118 |
| P1AJ0773 | 28.22 | 61.75 | 10.04 | 79.65 | 84 |
| P1AH4513 | 25.82 | 64.57 | 9.6 | 80.27 | 68 |
| P1AH6021 | 11.09 | 79.28 | 9.62 | 85.15 | 107 |

### Example 2.3. IL-7R Signaling (STAT5-P) on Activated PD-1+ and PD-1− CD4 T Cells Upon Treatment with Increasing Doses of Selected PD1-IL7 Double Mutants from Transient Expression

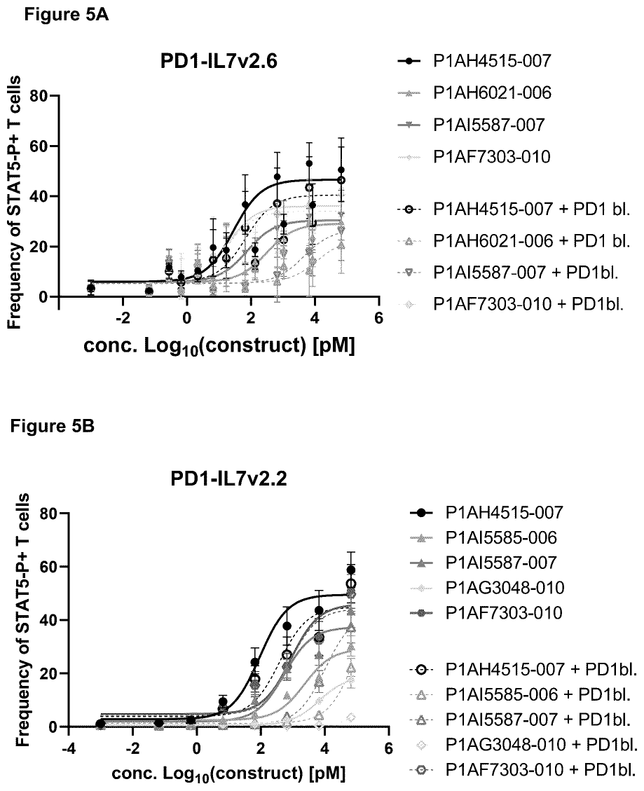

In the following experiment, the IL-7R signaling of two different PD1-IL7 double mutants were measured by exposing activated PD1⁺ and PD-1⁻ (anti-PD-1 pre-treated) CD4 T cells to increasing concentration of immunoconjugates. Given that PD1-IL7 wt acts in a PD1-independent fashion or in trans, meaning that the IL-7 wt moiety can signal in PD1 negative T cells cultured in close proximity to PD1⁺ cells, this experiment would allow the selection of PD1-IL7 mutants with reduced affinity to the IL7R to ensure the preferential signaling in cis, meaning that the IL-7v moiety mainly binds and signals through the IL-7R on the same PD-1⁺ Tcell after PD-1 docking. For this purpose, CD4 T cells were sorted from healthy donor PBMCs and the experiment was performed as described for Example 1. The data in the FIG. 5A-B show the potency difference of the two novel double mutant PD1-IL7 variants on PD-1⁺ and PD-1⁻ (pre-blocked cells with competing anti-PD-1 antibody) CD4 T cells. The potency measurement on the PD1⁺ CD4 T cells reflects the PD1-mediated delivery of IL-7R agonism versus the PD1-independent delivery of IL-7 in PD1 CD4 T cells. Tables 11 and 12 show the EC50, EC50 fold increase (cis-delivery and potency reduction) and Area under the Curve of the dose-response STAT-5 phosphorylation for the selected mutants, PD1-IL7 wt and FAP-IL7 wt on PD-1⁺ and PD-1⁻ CD4 T cells obtained from 5 donors.

The dose-response curves on PD-1⁺ T cells provide information on the potency of the single and double mutants in signaling through the IL-7R through PD-1 mediated delivery. In addition, the dose-response curves on T cells pre-treated with a competing anti-PD-1 antibody, to prevent the PD-1 mediated delivery, show the potency of the single and double mutants in providing IL-7R signalling independently from the binding to PD-1. In this particular assay P1AH6021 and P1AI5587 have, respectively, 38 and 60 folds reduced potency on PD-1⁻ T cells than on PD-1⁺ T cells, indicative of their preferential cis-activity (FIG. 5A) and that the P1AI5587 is more potent than the P1AH6021. Of note, P1AI5587 is only 2.6 less potent than P1AH4515 on PD-1⁺ T cells while it is 66 fold less potent on PD-1⁻ T cells (FIG. 5A and Tables 11 and 12). In FIG. 5B, P1AI5587 and P1AI5585 (derived from P1AH6005) are compared to P1AG3048, P1AH4515 and P1AF7303. The P1AI5587, P1AI5585 and P1AG3048 show an increasingly higher cis-activity window starting from 20 fold and the highest delta AUC between PD-1⁺ and PD-1⁻ T cells, also used to measure the cis-activity window, for P1AI5587 (FIG. 5B and Tables 11 and 12).

TABLE 11
EC50 and Area under the Curve (AUC) of the dose-response STAT-5 phosphorylation
for each mutant on PD-1⁺ and PD-1⁻ CD4 T cells obtained from 5 donors.

| | | | Cis-delivery: | | | | |
|---|---|---|---|---|---|---|---|
| | | | fold increase in | | | | |
| | PD1+ | PD1 blocked | EC50 | Potency reduction to | PD1+ | PD1bl. | |
| | (EC50) | (EC50) | EC50 [with | PD1-IL7wt: EC50 | (AUC) | (AUC) | |
| | (Mean ± | (Mean ± | PD1bl]/EC50 | (PD1+)/ EC50 (PD1- | (Mean ± | (Mean ± | delta |
| Batch ID | SEM) | SEM) | [no PD1bl] | IL7wt) | SEM) | SEM) | AUC |
| P1AH4515- | 28.5 | 67.6 | 2.4 | 1 | 139.1 | 110.1 | 29 |
| 007 | | | | | | | |
| P1AH6021- | 306.7 | 11646 | 38 | 10.8 | 74.7 | 42.4 | 32.4 |
| 006 | | | | | | | |
| P1AI5587- | 75.1 | 4472 | 59.6 | 2.6 | 91.4 | 50.7 | 40.7 |
| 007 | | | | | | | |
| P1AF7303- | 18.4 | 17.2 | 0.9 | 0.6 | 117.3 | 110 | 7.3 |
| 010 | | | | | | | |

TABLE 12
EC50 and Area under the Curve (AUC) of the dose-response STAT-5 phosphorylation
for each mutant on PD-1⁺ and PD-1⁻ CD4 T cells obtained from 6 donors.

| | | | Cis-delivery: | | | | |
|---|---|---|---|---|---|---|---|
| | | PD1 | fold increase in | | | | |
| | PD1+ | blocked | EC50 | Potency reduction to | PD1+ | PD1bl. | |
| | (EC50) | (EC50) | EC50 [with | PD1-IL7wt: EC50 | (AUC) | (AUC) | |
| | (Mean ± | (Mean ± | PD1bl]/EC50 | (PD1+)/EC50 (PD1- | (Mean ± | (Mean ± | delta |
| Batch ID | SEM) | SEM) | [no PD1bl | IL7wt | SEM) | SEM) | AUC |
| P1AH4515- | 92.9 | 389.6 | 4.2 | 1 | 141.2 | 109 | 32.2 |
| 005 | | | | | | | |
| P1AI5585- | 2295 | 110689 | 48.2 | 24.7 | 49 | 16.8 | 32.1 |
| 006 | | | | | | | |
| P1AI5587- | 540.3 | 10519 | 19.5 | 5.8 | 79.6 | 38.3 | 41.3 |
| 007 | | | | | | | |
| P1AJ0773- | 6670 | 31744011 | 4759.2 | 71.8 | 20 | 2.7 | 17.3 |
| 005 | | | | | | | |
| P1AF7303- | 872.9 | 819.3 | 0.9 | 9.4 | 102.6 | 99.6 | 3 |
| 010 | | | | | | | |

### Example 2.4: Rescue of T_{conv} effector function from T_{Reg} Suppression Upon PD1-IL7v Treatment from Transient Expression

In this assay it was assessed whether PD1-IL7 mutants can reverse the T_{reg} suppression of T_{conv} effector functions. Therefore, a suppressive-function assay was established, and for this purpose, T_{conv} and T_{reg} were isolated and labelled as described in Example 2.

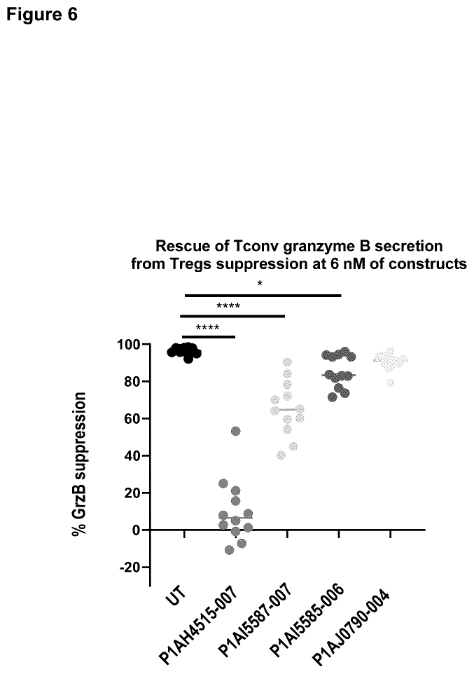

FIG. 6 shows the T_{reg} mediated suppression of granzyme B production by T_{conv} in a five days coculture in presence or absence of selected PD1-IL7 variants. Table 13 shows the percentage of T_{reg}-mediated suppression of granzyme B production by T_{conv} and the percentage of rescued granzyme B secretion by T_{conv} from T_{reg} suppression in presence of 6 nM of the different immunoconjugates. The data were obtained with T cells from twelve different donors. In the untreated samples, 96.4% of granzyme B secretion by T_{conv} was suppressed by T_{reg}. PD1-IL7 wt was used as positive control due to its ability to overcome T_{reg} suppression which in this case was reduced to 10.2% resulting in the ability to rescue T_{conv} granzyme B production to 89.8%. In the coculture treated with P1AI5587 and the P1AI5585 T_{reg} suppression was reduced to 65% and 85%, respectively, resulting in 34.5% and 14.7% rescue of T_{conv} effector functions. Of interest, an untargeted version of P1AI5587 only reduced T_{reg} suppression to 91%, equivalent to a rescue of 9% of T_{conv} ability to secrete granzyme B (FIG. 6 and Table 13).

TABLE 13
Percentage of T_{reg}-mediated suppression of granzyme
B produced by T_{conv} and percentage of rescued granzyme B
production by T_{conv}. Median of 12 donors.

| | | Percentage of | Percentage of |
|---|---|---|---|
| | Concen- | suppressed | rescued |
| | tration | Granzyme B | Granzyme B |
| Tapir-ID | (nM) | production by T_{conv} | production by T_{conv} |
| UT | | 96.4 | 3.6 |
| P1AH4515-007 | 6 | 10.2 | 89.8 |
| P1AI5587-007 | 6 | 65.4 | 34.6 |
| P1AI5585-006 | 6 | 85.3 | 14.7 |
| P1AJ0790-004 | 6 | 90.8 | 9.2 |

### Example 3

### Example 3.1 Production of IgG-Like Proteins in Stably-Transfected CHO Cells

A selection of variants was stably transfected into CHO cells to gain insights on parameters, like product titer, monomer content and product-related impurity profile in a fed-batch production process.

### Example 3.2. Generation of Stable CHO Producer Pools

Stable pools of CHO cells producing the selected PD1-IL7v variants were generated by double recombination-mediated cassette exchange (RMCE) of PD1-IL7v-coding transgenes into a pre-engineered genomic locus of a CHO expression host (TI host) using the Cre/lox recombinase system. The pre-engineered genomic locus, also called landing pad, is flanked by two heterospecific Lox sites: 2 L and L3. A third incompatible Lox site, LoxFas, is located inside the landing pad. The landing pad expresses EGFP for monitoring of a successful gene integration.

For stable transfection, this target region is replaced by a multi-gene expression cassette through double recombinase mediated-cassette exchange (RMCE). The final expression cassette is formed through recombination, when two plasmids (Plasmid 1 and Plasmid 2) are transfected into the TI host along with another plasmid directing the transient expression of Cre recombinase. DNA that is intended for integration is flanked by L3 and LoxFas in Plasmid 1, and by LoxFas and 2 L in Plasmid 2. Together with PD1-IL7v-encoding transgenes, a marker gene for positive selection is integrated into the target locus replacing the EGFP. The complete marker gene is assembled from one part of Plasmid 1, donating the promoter and the start codon, and a second part from Plasmid 2, donating the marker gene coding region and the polyadenylation signal. By this, only cells having integrated the intended parts of both plasmids survive the selection process. In order to optimize the expression of PD1-IL7v variants in stably transfected cells, two versions of Plasmid 1 and three versions of Plasmid 2 were tested in three different combinations (Table 14). The plasmids differ in the number and/or the order of the transgenes coding for the three different subunits of PD1-IL7v variants, which are H (heavy chain with hole mutation), K (heavy chain with knob mutation) and L (light chain).

### Example 3.3. Cloning of Plasmids for Targeted Integration

DNA fragments coding for PD1-IL7v subunits H, L and K were chemically synthesized and separately introduced into shuttle plasmids using suitable restriction sites thereby yielding the complete transgene for the subunits H, K or L. Each transgene comprises the following functional elements:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
the human gastrin terminator (hGT).

The transgenes were excised from their shuttle plasmids in a way that they were provided with suitable sticky ends. The Plasmids 1 and 2 were generated by combining DNA fragments carrying transgenes of H, K and L and another fragment carrying the bacterial backbone element (origin of replication and beta-lactamase resistance gene) in a multiway ligation.

TABLE 14
PD1-IL7 variants and gene configurations
utilized for stable expression.

| | | Gene configuration | |
|---|---|---|---|
| | PD1-IL7v | (Plasmid | Pool ID |
| Tapir-ID | variant | 1//Plasmid 2) | TAPIR Biosys |
| P1AI5585 | PD1-IL7v2.2 | L-K // H-L | PD1IL7v.S.0122 |
| | | K-L // L-H | PD1IL7v.S.0123 |
| | | L-K // L-H-K | PD1IL7v.S.0124 |
| P1AI5587 | PD1-IL7v2.6 | L-K // H-L | PD1IL7v.S.0125 |
| | | K-L // L-H | PD1IL7v.S.0126 |
| | | L-K // L-H-K | PD1IL7v.S.0127 |

### Example 3.4 Cultivation, Transfection and Selection of Stable CHO Pools

CHO host cells were propagated in disposable 125 ml vented shake flasks under standard humidified conditions (80% rH, 37° C., and 5% CO₂) at a constant agitation rate of 150 rpm. Every 3 to 4 days the cells were seeded at a concentration of 4×10e5 cells/ml in a proprietary, chemically-defined medium. Density and viability of the cultures were monitored with a Cedex HiRes cell counter (F. Hoffmann-La Roche Ltd, Basel, Switzerland). For stable transfection, CHO cells were seeded in fresh medium with a density of 4×10e5 cells/ml. Two days later, cells were transfected with Plasmids 1 and 2 together with a plasmid for a transient expression of Cre recombinase at a molar ratio of 5:5:2 using the MaxCyte STx device (MaxCyte, USA), according to the manufacturer's protocol, 3×10⁷ cells were transfected with 30 μg plasmid. After transfection the cells were seeded in 30 ml medium without selection agents. On day 5 after seeding, the cells were centrifuged and transferred to 80 mL seeding medium at 6×105 cells/ml for selection of recombinant cells. The seeding medium contained selection Agent 1 for negative selection and selection Agent 2 for positive selection at effective concentrations. Cell density and viability of the culture was monitored regularly. When the viability of the culture started to increase again, the concentrations of selection Agents 1 and 2 were reduced to about half the amount used before. About 28 days after the selection had been started, the viability exceeded 99% and selection was considered as complete. 29 days after starting the selection, the success of Cre mediated cassette exchange was checked by flow cytometry measuring the expression of intracellular EGFP and extracellular CrossMab sticking to the cell surface. An APC antibody (allophycocyanin-labeled F(ab′)2 Fragment goat anti-human IgG) against human antibody light and heavy chains was used for staining. Flow cytometry was performed with a MACSQuant® X Flow Cytometer (Miltenyi Biotec, Germany). 10,000 events per sample were measured. Living cells were gated in a plot of forward scatter (FSC) against side scatter (SSC). The live cell gate was defined with non-transfected host cells and applied to all samples by employing the FlowJo 10.8.1 EN software (TreeStar, Olten, Switzerland). Fluorescence of GFP was quantified in the FITC channel (excitation at 488 nm, detection at 530 nm) whereas PD1-IL7v was measured in the APC channel (excitation at 645 nm, detection at 660 nm). Subsequently, the cells were cryopreserved for later use.

### Example 3.5 Production

Stable transfection pools, generated as described above, were used to express proteins of interest in a fed-batch process. For a fed-batch process, cells were initially expanded in a proprietary, chemically-defined selective cell culture medium in disposable 125 ml vented shake flasks under standard humidified conditions (80% rH, 36.5° C., and 5% CO₂) at a constant agitation rate of 150 rpm. Every 3 to −4 days the cells were seeded at a defined cell density of 3-6×10e5 cells/ml. After 2 to 3 passages, cells were then split into a proprietary, chemically-defined non-selective cell culture medium at increasing cell densities, to accumulate sufficient biomass for inoculating of a fed-batch production process. For the fed-batch process, laboratory-scale bioreactors were filled with the non-selective fermentation medium and inoculated at a defined cell density. During the fed-batch cultivation a constant temperature, dissolved oxygen and pH were maintained. To avoid nutrient limitation, nutrient feeds wereadded as required. To isolate proteins, cell culture fluid was centrifuged to separate cells from the supernatants which were then filtered using 0.2 μm filter. All pools exhibited an analogous performance, achieving similar titers in the range of 3.8 to 4.0 g/L, as detailed in Table 15.

### Example 3.6. Small Scale Purification and Analytics of IgG-Like Proteins

Immunoconjugates were purified from cell culture supernatants by affinity chromatography using MabSelectSure-Sepharose™ (GE Healthcare, Sweden) chromatography. Purification was performed with Roboclumns® (Repligen, USA) on a Tecan Evo freedom liquid handling system. Briefly, sterile filtered cell culture supernatants were captured on a MabSelect SuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM citrate, pH 3.0. After neutralization with 1 M Tris pH 9.0, aggregated protein was separated from monomeric antibody species by size exclusion chromatography (Superdex 200, GE Healthcare) in 20 mM histidine, 140 mM NaCl, pH 6.0. Monomeric protein fractions were pooled and stored at −80° C. Product quality was analyzed by SEC using TSKgel UP-SW3000 (running buffer 200 mM K₂HPO₄/KH₂PO₄, 250 mM KCl, pH 6.2) and CE-SDS using LabChip® HT Protein Express Chip and LabChip® GXII protein characterization system (Perkin Elmer) applying the standard protocol for proteins.

TABLE 15
Summary of titer and CMC characteristics for various PD1-IL7 variant pools

| | | | | | CE-SDS (non-reduced) | | |
|---|---|---|---|---|---|---|---|
| | | SEC | | | Main peak | Hole-hole | Knob-knob |
| Pool ID TAPIR | Titer | Monomer | HMW | LMW | (%) 195 | (%) 172 | (%) 224 |
| Biosys | (g/L) | (%) | (%) | (%) | kDa | kDa | kDa |
| PD1IL7v.S.0122 | 4.0 | 49 | 29 | 22 | 66 | 12 | 2 |
| PD1IL7v.S.0123 | 3.8 | 41 | 41 | 18 | 65 | 4 | 5 |
| PD1IL7v.S.0124 | 4.8 | 41 | 43 | 17 | 61 | 4 | 8 |
| PD1IL7v.S.0125 | 4.2 | 69 | 12 | 19 | 85 | 8 | 2 |
| PD1IL7v.S.0126 | 3.9 | 51 | 26 | 22 | 71 | 4 | 5 |
| PD1IL7v.S.0127 | 4.5 | 51 | 24 | 25 | 66 | 3 | 7 |

All pools, regardless of the IL7 variant expressed or the gene configuration used (Table 14) demonstrated comparable titers (Table 15). Variant P1AI5585 showed a lower monomer content than P1AI5587, irrespective of the used gene configuration. Pool PDIIL7v.S.0122 exhibited the highest monomer content for P1AI5585, Pool PD1IL7v.S.0125 had the highest monomer content for P1AI5587 (Table 15). In both instances, the same gene configuration of L-K//H-L was represented in these pools. Consequently, the pools PD1IL7v.S.0122 and PD1IL7v.S.0125 were selected for P1AI5585 and P1AI5587, respectively, for further purification.

### Example 4

### Example 4.1 Purification of IgG-Like Proteins

120 ml of supernatants from pool PD1IL7v.S.0122 and PD1IL7v.S.0125 were purified via a 3-step purification using a Protein A chromatography step (ProA) for capturing and initial removal of process related impurities like host cell proteins and DNA. The following cation exchange chromatography step (CEX) increased the SE-HPLC monomer purity from roughly 50% after Protein A for PDIIL7v.S.0122 and 70% for PDIIL7v.S.0125 to approximately 90%. Hereby, product related impurities like aggregates, mispaired- and incomplete antibodies were removed. After centrifugal concentration to increase the protein concentration a final polishing step by size exclusion chromatography (SEC) was done. Thereby, the majority of all remaining process- and product related impurities was removed leading to a final SE-HPLC monomer purity of more than 96% for PD1IL7v.S.0122 and 98% for PD1IL7v.S.0125. (Table 16). Consequently, from 120 ml of supernatant, a total of 75.5 mg of the pure compound P1AI5585 and 117.5 mg of the pure compound P1AI5587 were obtained. The material obtained following size exclusion chromatography was used for further analysis.

TABLE 16
Cumulative yield and CMC characteristics for the pools PD1IL7v.S.0122 and PD1IL7v.S.0125

| | | | | | | | | | | |
|---|---|---|---|---|---|---|---|---|---|---|
| | Cumulative | | | | | | | | | |
| Purification | Yield | HMW4 | HMW3 | HMW2 | HMW1 | Monomer | LMW1 | LMW2 | LMW3 | LMW4 |
| Step | (%) | (%) | (%) | (%) | (%) | (%) | (%) | (%) | (%) | (%) |

Pool ID: PD1IL7v.S.0122 (P1AI5585; PD1-IL7v2.2)

| | | | | | | | | | | |
|---|---|---|---|---|---|---|---|---|---|---|
| ProA | 70 | 10.24 | 10.33 | — | 3.01 | 57.46 | 12.50 | — | 1.47 | 5.00 |
| CEX | 26 | 6.32 | 9.11 | — | 4.02 | 74.83 | — | — | — | 0.79 |
| SEC | 17.5 | — | 0.42 | — | 2.58 | 96.14 | — | 0.86 | — | — |

Molecule PD1IL7v.S.0125 (P1AI5587; PD1-IL7v2.6)

| | | | | | | | | | | |
|---|---|---|---|---|---|---|---|---|---|---|
| ProA | 95 | 3.52 | 5.02 | — | 2.27 | 74.05 | 7.35 | 2.89 | 1.24 | 3.64 |
| CEX | 44 | 3.36 | 3.12 | — | 2.33 | 89.13 | 1.53 | — | — | 0.52 |
| SEC | 34 | — | 0.12 | — | 0.68 | 98.09 | 0.74 | 0.36 | — | — |

### Example 4.2: Analysis of N-Glycan Pattern by 2-AB-Labeling of Released Oligosaccharides and HILIC Chromatography

Fully purified PD1-IL7v batches P1AI4485-407 and P1AI5587-468 from example 4.1 and batch P1AI4487-270 from a transient expression in CHO were characterized regarding their overall N-glycosylation pattern, i.e. deriving from Fc- and IL7v glycosylation by HILIC chromatography of released 2AB-labeled N-glycans. Enzymatic release of N-glycans, labeling with 2-Ab reagent and chromatographic separation of labeled glycans was performed as following:

TABLE 17
Equipment and reagents

| | |
|---|---|
| Instrumentation | Dionex Ultimate 3000 equipped with a fluorescence detector (FLD), a column heater |
| | capable and an autosampler with cooling capability |
| Column | Waters Acquity BEH Glycan Column, 2.1 × 150 mm, 1.7 μm |
| Further Devices | NanoSep ® Centrifugal Devices 10 K Omega (Pall Life Science) |
| | HyperSep-96 diol cartridge (60300-635, Thermo Scientific) or GlycoClean TM S-plus |
| | Cartridges, Glyko No.: GC210 (365) |
| | Clean-Up Station, Product Code: Glyko GC100 (connected to a vacuum source) (sealing |
| | plugs inclusive) |
| Reagents | N-glycosidase F (PNGase F) (Roche)(glycerol-free, # 11 365 185 001) |
| | Digestion Buffer (10 mM ammonium formate pH 8.6) |
| | Signal 2-AB-plus Labelling Kit, Prozyme GKK-804 |
| | Trypsin (sequencing grade modified), Prozyme V511B |
| | Resuspension buffer (Trypsin) supplied with V511B |

### Sample Preparation

200 μg of each sample was filled into NanoSep® Centrifugal Devices 10 K. Buffer exchange into digestion buffer (10 mM ammonium formate pH 8.6) was performed by 3x centrifugation down to almost dry and refilling each with 350 μL. After the final centrifugation step, 48 μL digestion buffer, 2 μL N-glycosidase F (PNGase F, glycerol-free, Roche, Cat #11 365 185 001) and 20 μL trypsin solution (1.0 mg/mL in resuspension-buffer, Prozyme V511B) were added and incubated in the NanoSep unit at 37° C. for 16-18 hours (overnight). N-linked oligosaccharides released from the Fc-part and the IL7 moiety were collected from the NanoSep unit into 1.5 mL Eppendorf screw cap tubes by flow-through centrifugation. 2-AB labeling of the released N-glycans was performed with the Signal 2-AB-plus Labelling Kit (Prozyme GKK-804) according to supplier's instructions (note: reaction has to occur in the dark). For cleaning of the 2-Ab labelled N-glycans, HyperSep-96 diol cartridges were prepared by equilibrating with 1 mL of water, followed by 1 mL of 96% (v/v) acetonitrile on a Glyko Clean-Up Station by applying vacuum (unused wells were blocked with strips of sealing plugs). 2-AB labelled N-glycan samples were mixed with 1 mL of 96% (v/v) acetonitrile and loaded onto the equilibrated HyperSep-96 diol cartridges and a very low vacuum was applied. The cartridge was washed with 3×0.75 mL 96% (v/v) acetonitrile and samples were transferred from the HyperSep-96 diol cartridges in 2 ml-centrifugal devices. 100 μL 20% (v/v) acetonitrile/water was added and penetration allowed for ˜ 2-3 minutes. Glycans were eluted by flow through centrifugation (˜ 2 min at 5000 rcf) (or by vacuum on the Glyko Clean-Up Station) and diluted 1:1 with 96% acetonitrile (v/v) for chromatographic analysis. 10 μL of each oligosaccharide sample was loaded onto the HILIC-BEH glycan column.

TABLE 18
Chromatograpic separation conditions

| | |
|---|---|
| Column temperature | 60° C. |
| Eluent system | |
| Eluent A | 100 mM ammonium formate pH 4.5 |
| Eluent B | 100% acetonitrile, Buffer A |
| Autosampler temperature: | 10° C. |
| Detection (Dionex-UPLC) | Fluorescence (λex = 330 nm; λem = 420 |
| | nm) |
| | Sensitivity: 6 |
| | Data Collection Rate: 5.00 Hz |
| | Response Time: 2 |

TABLE 19
Gradient applied to the HILIC-BEH glycan column

| Time | Flow | Eluent A | Eluent B |
|---|---|---|---|
| [min] | [ml/min] | [%] | [%] |
| 0 | 0.5 | 25 | 75 |
| 50 | 0.5 | 46 | 54 |
| 51 | 0.25 | 100 | 0 |
| 55 | 0.25 | 100 | 0 |
| 56 | 0.25 | 25 | 75 |
| 56.1 | 0.5 | 25 | 75 |
| 65 | 0.5 | 25 | 75 |

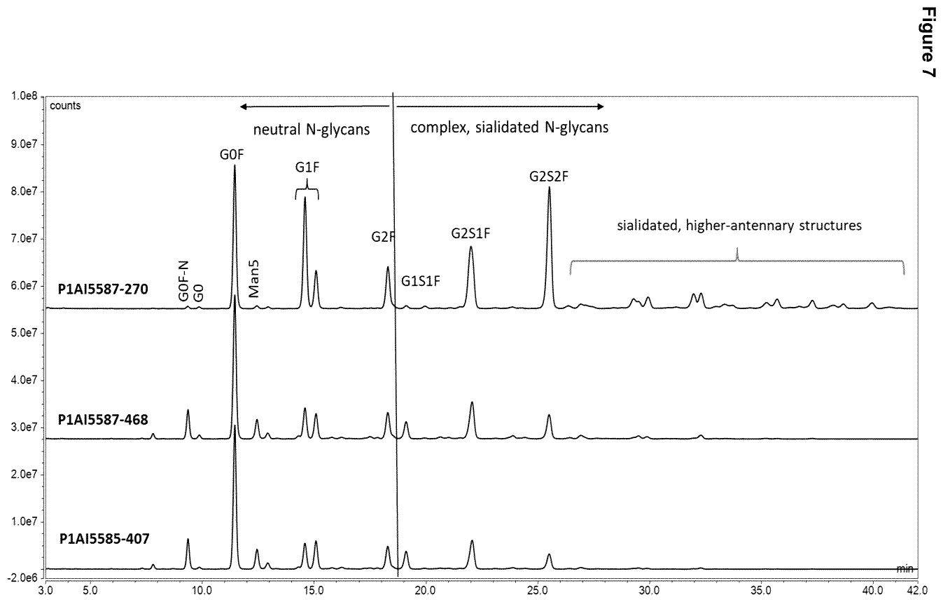

Batches of PD1-IL7v tested reveal N-glyco patterns as expected for a complex glycoprotein. PD1-IL7 batches expressed transiently or in stable transfected CHO cells showed significant complex, sialylated bi-, tri and tetra-antennary N-glycans at the IL7 part. Neutral non-charged N-glycans GOF, GIF and G2F mainly derived from Fc-glycosylation. Most of the charged bi-, tri- and tetra-antennary glycans are located at the IL7v part of the molecules. The ratios of individual N-glyco structures can be affected by the expression conditions as indicated by the different relative ratios of individual oligosaccharides or structure groups in the batches shown in FIG. 7.

### Example 4.3.1: Evaluation of Thermal Stability

Aggregation and melting (denaturation) propensity of fully purified samples of P1AI5585 and P1AI5587 was evaluated by simultaneous monitoring changes of light scattering and the intrinsic tryptophan fluorescence using an Uncle system (Unchained Labs) by stepwise ramping temperature. During the temperature increase the Uncle instrument measures static light scattering of the sample with 2 lasers at 266 (and 473 nm). In addition, tryptophanes within the protein are excited by the UV laser (266 nm) and the tryptophan emission spectrum is recorded.

9 μL of each protein at 1 mg/mL in 20 mM histidine, 140 mM NaCl, pH 6.0 are filled into Uni cuvettes and a linear thermal ramp from 30-90° C., with a ramp rate of 0.1° C./minute is applied. When the proteins start to aggregate, the intensity of scattered light strongly increases as larger molecules like oligomers or high molecular weight aggregates scatter light more efficiently than monomers. The aggregation onset temperature (Tagg) is defined as the temperature at which the scattering intensity starts to increase.

When a protein starts to denature, the protein unfolds and is changing its 3D structure. As a consequence buried tryptophanes become more solvent exposed, resulting in a change of the tryptophan emission spectrum at 330 nm and 350 nm. The melting temperature Tm, when the protein starts to unfold is calculated using the Barycentric Mean (BCM) analysis. The shift of the emission spectrum is used to determine the melting temperature Tm, when unfolding starts.

TABLE 20
Parameter determined to assess thermal stability

| Tapir-ID | PD1-IL7 variant | Tm | Tagg |
|---|---|---|---|
| P1AI5585 | PD1-IL7v2.2 | 62.8 | 62.5° C. |
| P1AI5587 | PD1-IL7v2.6 | 63.3 | 63.5° C. |

Purified protein of P1AI5585 and P1AI5587 showed a very similar behavior on aggregation onset (Tagg) and start of unfolding (Tm) in generic storage buffer at pH 6.0. No significant difference between both variants was detected.

### Example 4.3.2: Biochemical and Functional Stability Under Storage Stress

In order to test molecule stability regarding aggregation, fragmentation and functional binding purified material of P1AI5585 and P1AI5587 was stressed at 10 mg/mL for 2 weeks at 40° C. in generic storage buffer consisting of 20 mM His, 140 mM NaCl, pH 6.0 and for 2 weeks at 37° C. in PBS-buffer at physiological pH 7.4.

### Example 4.3.2.1: SEC and CE-SDS

Determination of the aggregate content was performed by HPLC chromatography at 25° C. using a flow rate of 0.3 mL/minute using an analytical size-exclusion column (TSKgel UP-SW3000, 4.6×300 mm, 2 μm) equilibrated in running buffer (200 mM KH₂PO₄, 250 mM KCl pH 6.2).

TABLE 21
SEC data of stressed and non-stressed
P1AI5585- and P1AI5587-PD1-IL7variants

| | | HMW | Monomer | LMW |
|---|---|---|---|---|
| Tapir-ID | Storage Condition | (%) | (%) | (%) |
| P1AI5585 | 2 w/pH 6.0, −80° C. | 2.1 | 96.9 | 1.0 |
| | 2 w/pH 6.0, +40° C. | 10.4 | 83.9 | 5.7 |
| | 2 w/pH 7.4 +37° C. | 10.5 | 84.1 | 5.3 |
| P1AI5587 | 2 w/pH 6.0, −80° C. | 0.4 | 99.5 | 0.1 |
| | 2 w/pH 6.0, +40° C. | 2.8 | 93.0 | 4.3 |
| | 2 w/pH 7.4 +37° C. | 5.0 | 90.4 | 4.7 |

P1AI5587 shows a significantly better stability profile compared to PIAI5585. At both stress conditions, monomer content of P1AI5585 decreased much higher (at pH 6.0 A13%, at pH 7.4 A12.8%) compared P1AI5587 (at ph 6.0 46.5%, at pH 7.4 49.2%). Both molecules show similar rates of fragmentation (generation of LMW species), but P1AI5587 reveals a better stability regarding aggregation.

TABLE 22
CE-SDS data (Caliper) of stressed and non-stressed
P1AI5585- and P1AI5587-PD1-IL7variants

| | | non-reduced | reduced | | |
|---|---|---|---|---|---|
| | | Main Peak | LC | HC | LC + |
| Tapir-ID | Stress Condition | (%) | (%) | (%) | HC (%) |
| P1AI5585 | 2 w/pH 6.0, −80° C. | 92.2 | 31.4 | 60.6 | 92.0 |
| | 2 w/pH 6.0, +40° C. | 88.7 | 32.6 | 56.9 | 89.5 |
| | 2 w/pH 7.4, +37° C. | 84.7 | 33.8 | 57.3 | 91.2 |
| P1AI5587 | 2 w/pH 6.0, −80° C. | 92.9 | 30.2 | 68.3 | 98.5 |
| | 2 w/pH 6.0, +40° C. | 90.4 | 30.7 | 66.8 | 97.5 |
| | 2 w/pH 7.4, +37° C. | 86.2 | 33.4 | 64.2 | 97.6 |

P1AI5587 (PD1-IL7v2.6) shows a better stability profile compared to P1AI5585 (PD1-IL7v2.2). At both stress conditions, degradation of P1AI5585 is higher (pH 6.0 413%, at pH 7.4 412.8%) than of P1AI5587 (at pH 6.0 46.5%, at pH 7.4 49.2%). Both molecules show similar rates of fragmentation, but P1AI5587 reveals a better stability regarding aggregation.

### Example 4.3.2: Functional Binding to Human PD1 and Human IL7Ralpha-IL2Rgamma-Fc Heterodimer by SPR

Changes in functional binding to the targets huPD1 and huIL7R after thermal stress was monitored by determining the relative active concentration (RAC), i.e. measuring the binding activity relative to non-stressed samples (=100%) on a Biacore T200 instrument (Cytiva). CM5 Sensor Chips and reagents HBS-N (0.1M HEPES, 1.5M NaCl, pH 7.4), Amino coupling Kit, immobilization buffer Acetate, pH 4.5 and HBS-P+ (0.01 M HEPES, 0.15 M NaCl, 0.005% v/v Surfactant P20, pH 7.4) were purchased from Cytiva and prepared as per supplier instructions. Capture antibody <PGLALA>, human huPD1-ECD_Fc (P1AD9704) and human IL7Rα-IL2Rg-Fc (P1AF4984) were prepared in house.

The <PGLALA> capture antibody was amino coupled on a CM5 Chip for 600s at a flow rate of 8 μL/min as per instruction provided by the supplier of the coupling kit. After the immobilization binding analysis was performed at 25° C. using HBS-P+ as running and dilution buffer. P1AI5585 and P1AI5587 samples to be analyzed were diluted to 1 μg/ml and binding to human huPD1-ECD_Fc (P1AD9704) and IL7Rα-IL2Rg-Fc (P1AF4984), resp., was determined. Assay linearity was confirmed during assay development by testing a dilution series of non-stressed PD1-IL7v samples covering 25%, 50%, 100% and 125% of the antibody testing concentration (1 μg/ml).

Binding to human PD1 was measured according to the cycle scheme in Table 23.

TABLE 23

| | | Association | Dissociation | Flow | Flow |
|---|---|---|---|---|---|
| Cycle command | Injected solution | time [s] | time [s] | [μl/min] | cell |
| Capture | PD1-IL7v samples | 60 | — | 5 | 2 |
| Target | huPD1-ECD-FC | 180 | 60 | 5 | 1, 2 |
| Regeneration | 10 mM NaOH | 2 × 30 s | — | 10 | 1, 2 |

Binding to human IL7Rα-IL2Rg-Fc was measured according to the cycle scheme in Table 24.

TABLE 24

| | | Association | Dissociation | Flow | Flow |
|---|---|---|---|---|---|
| Cycle command | Injected solution | time [s] | time [s] | [μl/min] | cell |
| Capture | PD1-IL7v samples | 30 | — | 5 | 4 |
| Target | IL7Ra-IL2Rg-Fc | 60 | 60 | 5 | 3, 4 |
| Regeneration | 10 mM NaOH | 2 × 30 | — | 10 | 3, 4 |

Relative active concentration (RAC) was calculated by referencing stressed samples to the respective unstressed sample (2 w/pH 6.0, −80° C.) for each target and each stressed antibody sample as 100% comparison. For the normalization of the protein concentration, reference is made to PGLALA-capturing (RAC values).

TABLE 25
Functional Binding of stressed and non-stressed PD1-
IL7variants P1AI5585- and P1AI5587- expressed as Relative
Active Concentration (RAC) determined by SPR

| | | Binding | Binding |
|---|---|---|---|
| Tapir-ID | Stress Condition | to huPD1 | to huIL7R |
| P1AI5585 | 2 w/pH 6.0, −80° C. | 100 | 100 |
| | 2 w/pH 6.0, +40° C. | 105 | 97 |
| | 2 w/pH 7.4 +37° C. | 113 | 104 |
| P1AI5587 | 2 w/pH 6.0, −80° C. | 100 | 100 |
| | 2 w/pH 6.0, +40° C. | 108 | 114 |
| | 2 w/pH 7.4 +37° C. | 114 | >125* |

*out of linear range

Conclusion: Stressed samples of P1AI5587 (PD1-IL7v2.6) as well as PIAI5585 (PD1-IL7v2.2) showed no decrease in binding relative to corresponding unstressed samples upon stress at pH 6.0 and/or pH 7.4 for 2 weeks at +40° C. neither to huPD1 nor to IL7-receptor complex. This indicates that no significant loss of function occurs at the harsh stress incubation applied. The increase of binding observed at some conditions for both molecules may be partially due to the assay variability and to the potential formation of functionally active HMW species binding with increased strength to the targets than monomer molecules.

### Example 4.4 Polyreactivity Assessment

Purified material of P1AI5585 and P1AI5587 was evaluated for its potential to bind non-specifically to diverse antigens/molecule classes. ELISA based polyreactivity assays were performed using 14 non-specific antigens (as listed in Table 26) and one specific antigen (human PD1-ECD-AviHis-Biotin, in house P1AA1435) serving as positive control. Maxi Sorp 384-well plates (Nunc, 464718) were coated with 20 μl antigen per well with antigen concentrations ranging between 0.1 and 2 μg/mL in PBS (10x, Roche, 11666789001) at 4° C. overnight. Between individual steps, plates were washed with 2x 90 μl PBST (1x PBS+0.1% Tween 20, Affimetrix/USB, 20605). Blocking was performed with 90 μl blocking buffer (2% BSA, Bovine Serum Albumin Fraction V, fatty acid free, Roche, 10735086001 in 1x PBS+0.2% Tween 20) for 1 h at room temperature without agitation. 25 μl of antibody samples with a concentration of 1 μg/mL in One Step ELISA buffer (OSEP, 1x PBS+0.5% BSA+0.05% Tween 20) were added and incubated at room temperature for 1h without agitation. Per well, 25 μl detection antibody (anti-human IgG Fc-specific; Jackson #109-036-098; dilution 1:7000 in OSEP) was added and incubated 1h at room temperature on a microplate shaker at 400 rpm. Detection substrate TMB (Roche Diagnostics GmbH, 11835033001) was added (25 μl/well, 4 min without agitation) and absorbance was measured at a wavelength of 370 nm and a reference wavelength at 492 nm using a Tecan Safire II ELISA reader. The raw measurement signals at 492 nm were subtracted from the respective raw measurement signals at 370 nm (absorbance correction). The absorbance corrected signals from the blank wells (detection antibody only) were subtracted from the absorbance corrected signals on the respective antigens (blank correction). All measurements were done in triplicates. Blank corrected signals were considered to indicate significant binding (scoring “Yes”) if they exceeded 5x the standard deviation of the blank signal over all non-specific antigens.

TABLE 26
Overview of non-specific antigens used for polyreactivity
testing and the respective scores for two PD1-IL7 variants.

| | | Score for | Score for |
|---|---|---|---|
| Unspecific antigen | Provider | P1AI5585 | P1AI5587 |
| Histone | Roche, 10223565001 | No | No |
| Parathyroid Hormone | US Biological, P3109-38B | Yes | No |
| Related Peptide (PTH-RP) | | | |
| (1-34) amide | | | |
| Streptavidin | Roche, 11721674001 | Yes | No |
| Hemocyanin (KLH) | Sigma-Aldrich, H7017 | Yes | No |
| PBS/BSA Block | Roche, 10735108001 | Yes | No |
| Insulin | Sigma-Aldrich, 10516 | Yes | No |
| CD40 ECD | SinoBiological, 10774-H08H | Yes | No |
| Apolipoprotein A-1 | Biomol, 99913.1 | Yes | No |
| HSP90 alpha | Abcam, 48801 | Yes | No |
| Cardiolipin | Sigma-Aldrich, 1649 | Yes | No |
| Heparan sulfate | Sigma-Aldrich, H9902 | Yes | No |
| DNA MB grade | Roche, 11467140001 | Yes | No |
| E. coli Lysate | In house | Yes | No |
| Gelatine 2% | Roche, 11111965001 | Yes | No |
| | Total Score | 13 | 0 |

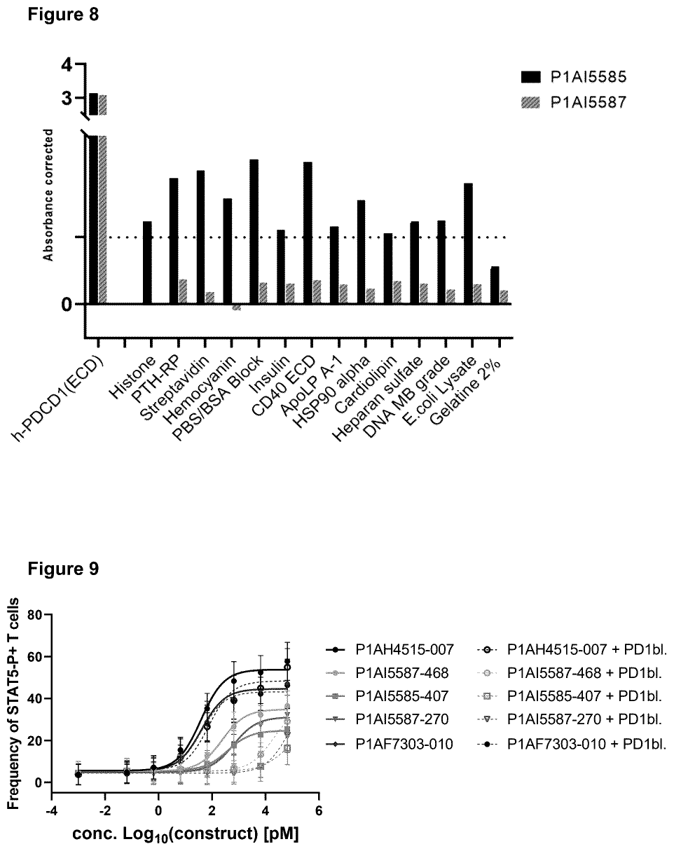

For P1AI5587 no significant binding to any nonspecific antigen could be detected while P1AI5585 showed significant binding to 13 out of 14 antigens based on the analysis as described above (Table 26, FIG. 8). This points toward strong unspecific binding of P1AI5585 in general. The two molecules P1AI5585 and P1AI5587, each containing two amino acid substitutions in the IL7, exhibit entirely distinct and unforeseeable responses in several aspects. These aspects include their monomer content after high titer recombinant expression in stably transfected CHO cells, their stability under stress conditions, and their nonspecific binding to unrelated antigens, known as polyreactivity. Even when considering previous data from single mutants, the outcomes from combining mutations remain unpredictable.

### Example 4.5 IL-7R Signaling (STAT5-P) on Activated PD-1+ and PD-1− CD4 T Cells Upon Treatment with Increasing Doses of Selected PD1-IL7 Double Mutants from a Stable Transfection Pool

In the following experiment, the IL-7R signaling of 2 different PD1-IL7 double mutants from stable transfection were measured and compared to a corresponding double mutant from transient expression and a competitor PD1-IL7 mutant by exposing activated PD1⁺ and PD-1⁻ (anti-PD-1 pre-treated) CD4 T cells to increasing concentration of immunoconjugates. Given that PD1-IL7 wt acts in a PD1-independent fashion or in trans, meaning that the IL-7 wt moiety can signal in PD1-negative T cells cultured in close proximity to PD1⁺ cells, this experiment allows for the assessment of preferential signaling in cis of the PD1-IL7 mutants, meaning that the IL-7v moiety mainly binds and signals through the IL-7R on the same PD-1⁺ T cell after PD-1 docking.

For this purpose, CD4 T cells were sorted from healthy donor PBMCs and the experiment was performed as described for Example 1.3. The data in the FIG. 9 show the potency difference of the two novel double mutant PD1-IL7 variants, from stable transfection, on PD-1+ and PD-1-(pre-blocked cells with competing anti-PD-1 antibody) CD4 T cells. The potency measurement on the PD1+CD4 T cells reflects the PDI-mediated delivery of IL-7R agonsim versus the PD1-independent delivery of IL-7R agonism in PD1− CD4 T cells. Table 27 shows the EC50, EC50 fold increase (cis-delivery and potency reduction) and Area under the Curve of the dose-response STAT-5 phosphorylation for the PIAI5587 and P1AI5585 from stable transfection, P1AI5587 from transient transfection, P1AH4515 and P1AF7303 on PD-1⁺ and PD-1⁻ CD4 T cells obtained from eight donors.

The dose-response curves on PD-1⁺ T cells provide information on the potency of the single and double mutants in signaling through the IL-7R. In addition, the dose-response curves on T cells pre-treated with a competing anti-PD-1 antibody to prevent the PD-1 mediated delivery show the potency of the single and double mutants in providing IL-7R signalling independently from the binding to PD-1. In this particular assay, the P1AI5587 and P1AI5585 from stable transfection have respectively, 80 and 120 folds reduced potency on PD-1⁻ T cells than on PD-1+ T cells, indicative of their preferential cis-activity (FIG. 9). The transiently expressed P1AI5587 is also 118 fold more potent on PD-1+ T cells than on PD-1− T cells. Of note, P1AI5587 from stable transfection is only 6 fold less potent than P1AH4515 (PD1-IL7 wt) on PD-1+ T cells while it is 250 fold less potent on PD-1− T cells (FIG. 9 and Table 27).

P1AF9658 shows a cis-delivery on PD-1+ T cells of only 2 folds higher than on PD-1− T cells, similarly to PD1-IL7 wt although being 16 folds less potent on PD-1+ T cells than PD1-IL7 wt (FIG. 9 and Table 27).

TABLE 27
EC50 and Area under the Curve (AUC) of the dose-response STAT-5 phosphorylation
for each mutant on PD-1⁺ and PD-1⁻ CD4 T cells obtained from 8 donors.

| | | | Cis-delivery: | | | | |
|---|---|---|---|---|---|---|---|
| | PD1+ | PD1blocked | fold increase in | Potency reduction | PD1+ | PD1bl. | |
| | (EC50) | (EC50) | EC50 | to PD1-IL7wt: EC50 | (AUC) | (AUC) | |
| | (Mean ± | (Mean ± | EC50 [with PD1bl]/ | (PD1+)/EC50 (PD1- | (Mean ± | (Mean ± | Delta |
| Batch ID | SEM) | SEM) | EC50 [no PD1bl] | IL7wt) | SEM) | SEM) | AUC |
| P1AH4515 | 38.5 | 76.4 | 2 | 1 | 168.1 | 137 | 31.1 |
| P1AI5587- | 236.4 | 19060 | 80.6 | 6.1 | 80.2 | 29.5 | 50.7 |
| 468 | | | | | | | |
| P1AI5585- | 386 | 46469 | 120.4 | 10 | 52.6 | 16.8 | 35.8 |
| 407 | | | | | | | |
| P1AF9658- | 617.4 | 1369 | 2.2 | 16 | 77.3 | 59.2 | 18.2 |
| 001 | | | | | | | |
| P1AI5587- | 642.7 | 76081 | 118.4 | 16.7 | 60.3 | 17.3 | 43 |
| 270 | | | | | | | |
| P1AF7303- | 40.2 | 39.9 | 1 | 1 | 136.9 | 132 | 4.9 |
| 010 | | | | | | | |

### Example 4.6 IL-7R Signaling (STAT5-P) on Activated PD-1+ Versus Freshly Isolated IL-7Ra+ CD4 T Cells Upon Treatment with Increasing Doses of PD1-IL7 Single and Double Mutants

To measure the on-target and off-target activity of the PD1-IL7 double mutants, the P1AK9505 (OSE PD1-IL7 mutant), P1AH4515 and P1AF7303, the IL-7R signaling in activated PD-1+, representing the desired target, versus freshly isolated IL-7Ra⁺ CD4 T cells, representing the peripheral sink for an IL-7 therapy, was measured upon exposure to increasing concentration of immunoconjugates.

For this purpose, CD4 T cells were sorted from healthy donor PBMCs and activated as described for Example 1. Three days later, the cells were harvested and washed several times to remove endogenous cytokines and the cells were labeled with CTV (5 μM, 5 min at RT; C34557, Thermo Scientific) and co-cultured 1:1 (25 μl, 6*106 cells/ml for each population) with freshly isolated CD4 T cells from an unrelated donor in a V-bottom plate before being treated for 12 min at 37° C. with increasing concentrations of treatment antibodies (50 μl, 1:10 dilution steps). To preserve the phosphorylation state, an equal amount of Phosphoflow Fix Buffer I (100 μl, 557870, BD) was added right after 12 minutes incubation with the various constructs. The cells were then incubated for additional 30 min at 37° C. before being permeabilized overnight at −80° C. with Phosphoflow PermBuffer III (558050, BD). On the next day STAT-5 in its phosphorylated form was stained for 30 min at 4° C. by using an anti-STAT-5P antibody (47/Stat5 (pY694) clone, 562076, BD). The cells were acquired at the FACS BD-Symphony A3/A5 (BD Bioscience). The frequency of STAT-5P was determined with FlowJo (V10) and plotted with GraphPad Prism (v8).

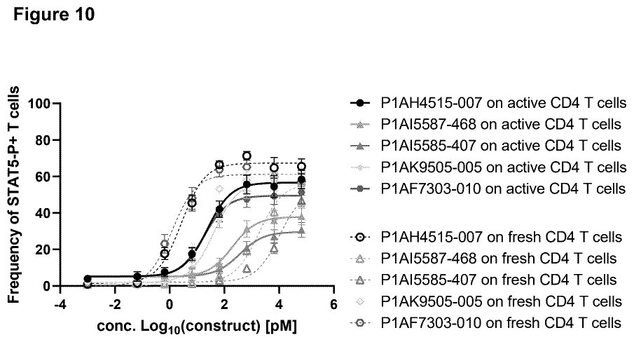

The dose-response curves on PD-1+ T cells provide information on the potency of the single and double mutants compared to PD1-IL7 wt in signaling through the IL-7R on target T cells. Conversely, the dose-response curves on freshly isolated T cells which express high levels of IL-7Ra, show the potency of the double mutants compared to PD1-IL7 wt or FAP-IL7 wt in providing IL-7R signaling on off-target peripheral T cells. In this particular assay P1AI5587 and P1AI5585 from stable transfection have, respectively, 682 and 3925 folds reduced potency on IL-7Ra⁺ T cells than P1AH4515 (PD1-IL7 wt), while having only a 8.3 and 15.3 fold reduction in potency on PD-1⁺ T cells (FIG. 10 and Table 28). Of relevance, PIAK9505 (OSE PD1-IL7 mutant) had only 5 fold less IL-7R signaling on IL-7Ra⁺ T cells associated with a 1.8 fold reduction in potency on PD-1+ T cells (FIG. 10 and Table 28). These data are indicative of the preferential activity of P1AI5587 and P1AI5585 from stable transfection on PD-1+ T cells due to the reduced affinity to the IL-7Ra and therefore reduced off-target effect.

TABLE 28
EC50 of the dose-response STAT-5 phosphorylation for each mutant on
activated and freshly isolated CD4 T cells obtained from six donors.

| | PD1+ | IL-7R+ | EC50[active]/ | EC50[fresh]/ |
|---|---|---|---|---|
| | (EC50) | (EC50) | EC50 [active | EC50 [fresh |
| Batch ID | (Mean ± SEM) | (Mean ± SEM) | PD1-IL7wt) | PD1-IL7wt] |
| P1AH4515 | 23.7 | 2.7 | 1 | 1 |
| P1AI5587-468 | 196.9 | 1840 | 8.3 | 681.5 |
| P1AI5585-407 | 362 | 10597 | 15.3 | 3924.8 |
| P1AK9505-005 | 41.5 | 13.4 | 1.8 | 5 |
| P1AF7303-010 | 18 | 1.3 | 0.8 | 0.5 |

### Example 4.7: Target Binding on CD4 and CD8 T Cell Subsets from Freshly Isolated PBMCs

In order to assess whether the potency reduction of the IL-7v associated with the PD-1 mediated delivery modify the target binding of P1AI5587 to different T cell populations, freshly isolated PBMCs from healthy donors were incubated with increasing concentrations of P1AH4515, P1AI5587, P1AK9505 (OSE PD1-IL7 mutant) and P1AF7303 (FAP-IL7 wt) (50 μl, 1:10 dilution steps with the top concentration of 66 nM) for 30 min at 4° C. The cells were then washed and stained on the surface with anti-human CD3, CD4, CD8, gdTCR, CD45RA, CD62L, TIM-3 and anti-PGLALA antibodies together with the Fixable Viability Dye eFluor™ 780 (eBioscience). PBMCs were next fixed/permeabilized with Fix/Perm Buffer (BD Biosciences) and incubated overnight at 4° C. The cells were later intracellularly stained in a step-by-step approach: first with anti-human endogenous PD-1 (Cell Signaling), than with an anti-rabbit secondary antibody and lastly with anti-human TCF-1 and FOXP-3 antibodies.

The cells were acquired at the FACS BD-Symphony A3/A5 (BD Bioscience). The frequency of anti-PGLALA⁺ was determined with FlowJo (V10) and plotted with GraphPad Prism (v8). CD4 and CD8 T cells were classified based on their expression of PD-1 and TCF-1 in naïve (TCF-1⁺ PD-1⁻), stem-like (PD-1⁺ TCF-1⁺), effector (PD-1⁺ TCF-1⁻) and bystander (PD-1⁻ TCF-1⁻), where naïve T cells and bystander T cells are not antigen-specific and therefore an unwanted off-target sink for IL-7 based therapies.

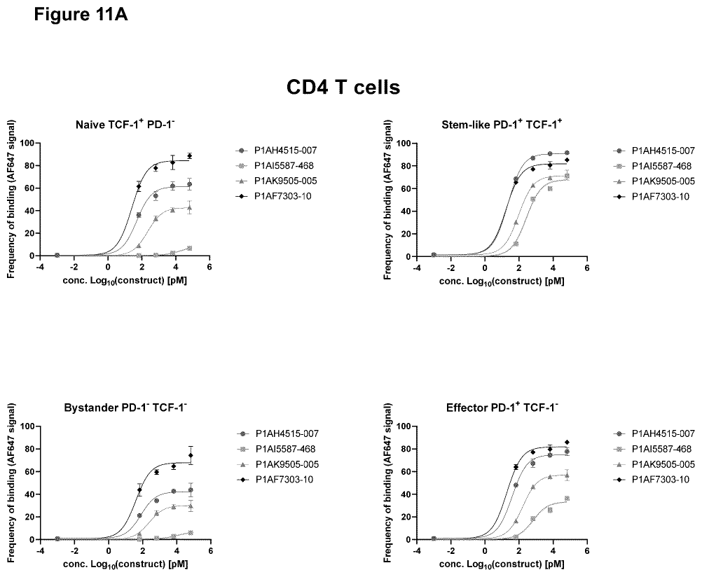

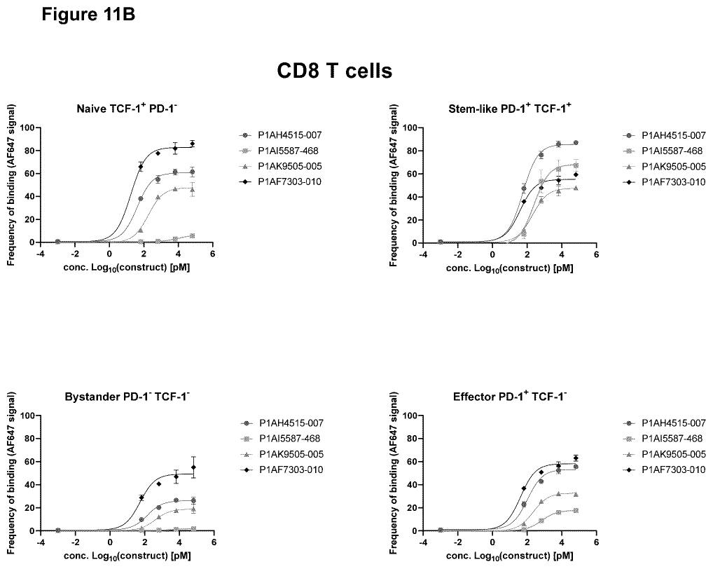

FIGS. 11A and 11B show the dose-response binding of the different constructs to naïve, stem-like, effector and bystander T cells within CD4 and CD8 T cells respectively. Tables 28, 29, 30 and 31 depict the AUC of the different immunoconjugates and fold reduction in binding to the different CD4 and CD8 T cell subsets compared to FAP-IL7 wt. P1AF7303 (FAP-IL7 wt) binds equally well across the different T cells subsets within the CD4 population, with a preference for naïve T cells in the CD8 population (FIG. 11A-B and Tables 29-31 7A-B). P1AH4515 (PD1-IL7 wt) shows a comparable binding behavior to P1AF7303 (FAP-IL7 wt) with a slight increased binding to stem-like T cells among CD4 and CD8 T cells, followed by P1AK9505 (OSE PD1-IL7 mutant) with 2-3.5 fold reduced binding to naïve and bystander CD4 T cells respectively (FIG. 11A and Tables 28-29) and 2.6-4.2 reduced binding to naïve, bystander and effector CD8 T cells (FIG. 11B and Tables 30-31). Conversely P1AI5587 has a 48 fold reduced binding to naïve and 30 fold reduced binding to bystander CD4 T cells which represent an important sink in the development of IL-7 based therapies, with only a 2.16 fold and 5.4 fold reduction in binding to stem-like and effector CD4 T cells (FIG. 11A and Tables 28-29). Within CD8 T cells where P1AF7303 (FAP-IL7 wt) showed an even stronger binding to the naïve subset, P1AI5587 has a 39.6 fold reduced binding to naïve, a 38 fold and a 6.5 fold reduced binding to effector and bystander T cells respecitvely, with an only 1.35 fold reduced binding to stem-like T cells (FIG. 11B and Tables 30-31.

Taken together these data show a preferential binding of P1AI5587 to stem-like CD4 and CD8 T cells due to the potency reduction of the IL-7v allowing for its PD-1 mediated delivery to cells which co-express PD-1 and IL-7R, rather than to cells which express IL-7R only and represent an off-target/sink for the therapy.

TABLE 28
AUC of the different immunoconjugates in binding
to the different CD4 and CD8 T cell subsets

Differently differentiated T-cell variants

| Tapir-ID | Naïve | Stem-like | Effector | Bystander |
|---|---|---|---|---|
| P1AF7303-010 | 385.1 | 394.8 | 388.3 | 290.5 |
| P1AH4515 | 252.8 | 426.4 | 322.2 | 162.2 |
| P1AK9505-005 | 122.2 | 260.4 | 180.7 | 84.0 |
| P1AI5587-468 | 8.0 | 182.8 | 71.7 | 9.8 |

TABLE 29
Fold reduction in binding to the different CD4
and CD8 T cell subsets compared to t P1AF7303

Differently differentiated T-cell variants

| Tapir-ID | Naïve | Stem-like | Effector | Bystander |
|---|---|---|---|---|
| P1AF7303-010 | 1.00 | 1.00 | 1.00 | 1.00 |
| P1AH4515 | 1.52 | 0.93 | 1.21 | 1.79 |
| P1AK9505-005 | 3.15 | 1.52 | 2.15 | 3.46 |
| P1AI5587-468 | 48.10 | 2.16 | 5.42 | 29.74 |

TABLE 30
AUC of the different immunoconjugates in binding to
the different CD4 and CD8 T cell subsets AUC Read Out

Differently differentiated T-cell variants

| Tapir-ID | Naïve | Stem-like | Effector | Bystander |
|---|---|---|---|---|
| P1AF7303-010 | 395.9 | 239.4 | 200.0 | 247.5 |
| P1AH4515 | 259.0 | 345.9 | 89.0 | 191.2 |
| P1AK9505-005 | 141.5 | 142.5 | 46.8 | 93.0 |
| P1AI5587-468 | 10.0 | 176.8 | 5.3 | 37.9 |

TABLE 31
Fold reduction in binding to the different CD4 and CD8 T cell
subsets compared to tReduction factor related to P1AF7303

Differently differentiated T-cell variants

| Tapir-ID | Naïve | Stem-like | Effector | Bystander |
|---|---|---|---|---|
| P1AF7303-010 | 1.00 | 1.00 | 1.00 | 1.00 |
| P1AH4515 | 1.53 | 0.69 | 2.25 | 1.29 |
| P1AK9505-005 | 2.80 | 1.68 | 4.28 | 2.66 |
| P1AI5587-468 | 39.60 | 1.35 | 37.99 | 6.53 |

### Example 5

### Example 5.1: In Vivo Efficacy of Murinized P1AI5587 (PD1-IL7v2.6) in a Syngeneic Model of Mouse Tumor Cell Line in Comparison to Pembrolizumab

Murinized P1AI5587 (PD1-IL7v2.6) corresponding to Tapir ID P1AL9335 was tested as single agent in comparison to murinized Pembrolizumab antibody (see SEQ ID NOs: 79 and 80 for the respective LC and HC sequences) for its anti-tumoral efficacy in one syngeneic model.

### Example 5.2: Panc02 Pancreatic Subcutaneous Syngeneic Model

The murinized version of P1AI5587 (PD1-IL7v2.6) was tested in the mouse pancreatic Panc02-H7 cell line subcutaneously injected into Black 6-huPD1-huIL2RBG tg mice in comparison to murinized standard of care Pembrolizumab.

Panc02-H7 cells (mouse pancreatic carcinoma) were originally obtained from the MD Anderson cancer center (Texas, USA) and after expansion deposited in the internal cell bank. Panc02-H7 cells were cultured in RPMI medium containing 10% FCS (Sigma), 500 μg/ml hygromicin and 1% of Glutamax. The cells were cultured at 37° C. in a water-saturated atmosphere at 5% CO2. Passage 15 was used for transplantation. Cell viability was 94.3%. 3×105 cells per animal were injected subcutaneously in 100 μl of RPMI cell culture medium (Gibco) into the flank of mice using a 1 ml tuberculin syringe (BD Biosciences).

Female Black 6-huPD1-huIL2RBG transgenics mice, aged 7-9 weeks at the start of the experiment (Charles Rivers, Lyon, France) were maintained under specific-pathogen-free condition with daily cycles of 12 h light/12 h darkness according to committed guidelines (GV-Solas; Felasa; TierschG). After arrival, animals were maintained for one week to get accustomed to the new environment and for observation. Continuous health monitoring was carried out on a regular basis.

Mice were injected subcutaneously on study day 0 with 3×105 of Panc02 cells, randomized and weighed. Twelve days after the tumor cell injection (tumor volume >200 mm3), mice were injected i.v. with murinized P1AI5587, murinized Pembrolizumab or vehicle once a week for two weeks. All mice were injected i.v. with 200 μl of the appropriate solution. The mice in the Vehicle group were injected with Histidine Buffer and the treatment groups with the murinized P1AI5587 with 2 mg/kg iv qw or murinized Pembrolizumab with 2 mg/kg iv qw for 2 weeks. To obtain the proper amount of antibodies per 200 μl, the stock solutions were diluted with Histidine Buffer when necessary. The tumor volume was measured using a caliper at study days 12, 14, 16, 19, 21, 23, 26 and 28.

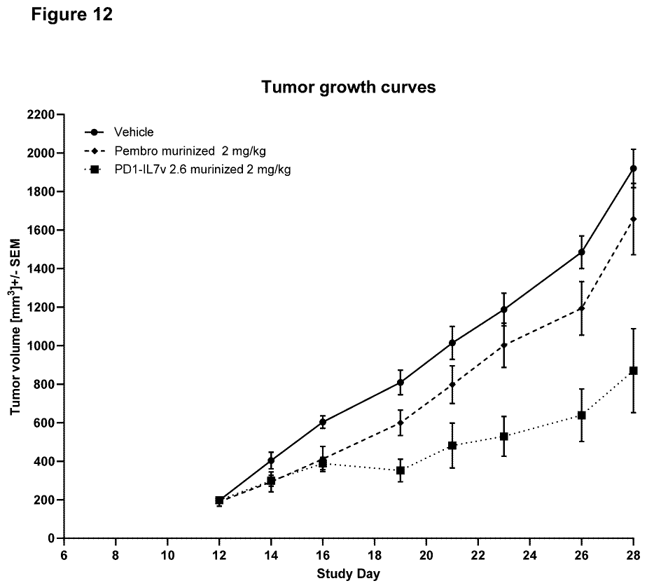

FIG. 12 shows that the murinized P1AI5587 mediated significantly superior efficacy in terms of tumor growth inhibition compared to the Vehicle and murinized Pembrolizumab groups.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A mutant interleukin-7 (IL-7) polypeptide comprising the amino acid substitutions E13K and G85E, wherein the numbering is relative to the human IL-7 sequence of SEQ ID NO: 64.

2. The mutant IL-7 polypeptide according to claim 1, wherein the binding affinity to IL-7Rα is reduced as compared to a reference IL-7 polypeptide which is identical to the mutant IL-7 except that it has E at position 13 and G at position 85, wherein the numbering is relative to the human IL-7 sequence of SEQ ID NO: 64.

3. The mutant IL-7 polypeptide according to claim 1, wherein the mutant IL-7 polypeptide comprises an amino acid sequence that is at least 95% identical to SEQ ID NO: 66.

4. An immunoconjugate comprising (i) the mutant IL-7 polypeptide of claim 1 and (ii) an antibody.

5. The immunoconjugate according to claim 4, wherein the antibody binds to PD-1.

6. The immunoconjugate according to claim 4, wherein the antibody comprises (a) a heavy chain variable region (VH) comprising a CDR-H1 comprising the amino acid sequence of SEQ ID NO:67, a CDR-H2 comprising the amino acid sequence of SEQ ID NO: 68, a CDR-H3 comprising the amino acid sequence of SEQ ID NO:69, and (b) a light chain variable region (VL) comprising a CDR-L1 comprising the amino acid sequence of SEQ ID NO: 70, a CDR-L2 comprising the amino acid sequence of SEQ ID NO:71, and a CDR-L3 comprising the amino acid sequence of SEQ ID NO:72.

7. An immunoconjugate according toclaim 6, wherein the antibody comprises (a) a heavy chain variable region (VH) comprising an amino acid sequence that is at least 95% identical to the amino acid sequence of SEQ ID NO: 73, and (b) a light chain variable region (VL) comprising an amino acid sequence that is at least 95% identical to the amino acid sequence of SEQ ID NO:74.

8. The immunoconjugate of claim 4, wherein the antibody comprises an Fc domain and the mutant IL-7 polypeptide is fused at its amino-terminal amino acid to the carboxy-terminal amino acid of one of the subunits of the Fc domain, optionally through a linker peptide.

9. The immunoconjugate of claim 8, wherein the linker peptide has the amino acid sequence of SEQ ID NO: 75.

10. The immunoconjugate of claim 4, comprising two polypeptides, each comprising an amino acid sequence that is at least 95% identical to the amino acid sequence of SEQ ID NO: 53, a polypeptide comprising an amino acid sequence that is at least 95% identical to the amino acid sequence of SEQ ID NO: 54, and a polypeptide comprising an amino acid sequence that is at least 95% identical to the amino acid sequence of SEQ ID NO: 57.

11. The immunoconjugate of claim 4, comprising two polypeptides, each comprising the amino acid sequence of SEQ ID NO: 53, a polypeptide comprising the amino acid sequence of SEQ ID NO: 54, and a polypeptide comprising the amino acid sequence of SEQ ID NO:57.

12. One or more isolated polynucleotides encoding the mutant IL-7 polypeptide of claim 1.

13. One or more vectors comprising the polynucleotide(s) of claim 12.

14. A host cell comprising the polynucleotide(s) of claim 12.

15. A method of producing a mutant IL-7 polypeptide comprising (a) culturing the host cell of claim 14 under conditions suitable for the expression of the mutant IL-7 polypeptide, and (b) recovering the mutant IL-7 polypeptide.

16. (canceled)

17. A pharmaceutical composition comprising (a) the mutant IL-7 polypeptide of claim 1 and (b) a pharmaceutically acceptable carrier.

18. (canceled)

19. (canceled)

20. A method of treating a disease in an individual, comprising administering to said individual a therapeutically effective amount of a composition comprising the mutant IL-7 polypeptide of claim 1 in a pharmaceutically acceptable form.

21. A method of stimulating the immune system of an individual, comprising administering to said individual an effective amount of a composition comprising the mutant IL-7 polypeptide of claim 1 in a pharmaceutically acceptable form.

22. (canceled)

23. One or more isolated polynucleotides encoding the immunoconjugate of claim 4.

24. One or more vectors comprising the polynucleotide(s) of claim 23.

25. A host cell comprising the polynucleotide(s) of claim 23.

26. A method of producing an immunoconjugate comprising a mutant IL-7 polypeptide and an antibody that binds to PD-1, comprising (a) culturing the host cell of claim 25 under conditions suitable for the expression of the immunoconjugate, and (b) recovering the immunoconjugate.

27. A pharmaceutical composition comprising (a) the immunoconjugate of claim 4 and (b) a pharmaceutically acceptable carrier.

28. A method of treating a disease in an individual, comprising administering to said individual a therapeutically effective amount of the immunoconjugate of claim 4 in a pharmaceutically acceptable form.

29. A method of stimulating the immune system of an individual, comprising administering to said individual an effective amount of a composition comprising the immunoconjugate of claim 4 in a pharmaceutically acceptable form.
